(19) United States
(12) Patent Application Publication
(10) Pub. No.: US 2024/0124661 A1
(43) Pub. Date: Apr. 18, 2024

(54) METHOD AND APPARATUS FOR DESIGNS, MATERIALS, AND METHODS OF MANUFACTURING COMPOSITE MATERIALS AND IMPLANTS

(71) Applicant: 206 ORTHO, INC., Deerfield, NH (US)

(72) Inventors: Jeffrey A. D'Agostino, Deerfield, NH (US); Stephen S. Keaney, Groton, MA (US); Elizabeth Nelson, Wellesley, MA (US)

(21) Appl. No.: 17/776,581

(22) PCT Filed: Nov. 13, 2020
(86) PCT No.: PCT/US2020/060612

§ 371 Date: May 12, 2022

Related U.S. Application Data
(60) Provisional application No. 62/935,001, filed on Nov. 13, 2019.
(60) Provisional application No. 63/093,659, filed on Oct. 19, 2020.

(51) Int. Cl. C08J 5/08; C08J 5/04; C08J 5/24

(52) U.S. Cl. CPC C08J 5/08; C08J 5/046; C08J 5/246; C08J 2375/04

(57) ABSTRACT

Composite parts (e.g. for implants) comprising fiber bundles including a plurality of aligned inorganic filaments, which are coated or impregnated with a polymer, and a polymeric matrix. The composite is stiff and ductile and preferably biodegradable and/or bioresorbable.

### REFERENCE TO RELATED PATENT APPLICATIONS

This patent application is related to U.S. Provisional Patent Application No. 62/935,001 (filed Nov. 13, 2019) and U.S. Provisional Patent Application No. 63/093,659 (filed Oct. 19, 2020) which are incorporated herein by reference each in its entirety.

### FIELD

This invention relates to designs, materials, and methods of manufacturing composite materials comprising hierarchical structures that integrate architectural elements across multiple and within scale lengths.

In addition, this invention relates to methods and apparatus for treating bones, and more particularly to methods and apparatus for treating bone fractures and/or for fortifying and/or augmenting bone in mammals.

In addition, this invention relates to methods and apparatus for treating bones, and more particularly to methods and apparatus for treating bone fractures and/or for fortifying and/or augmenting bone in mammals, and relates to novel composite structures which may be used for medical and non-medical applications.

### BACKGROUND

Conventional materials are subject to a number of trade-offs with respect to their properties. Rigid materials (e.g., higher Young's modulus) tend to exhibit brittle failure while elastic materials (e.g., lower Young's modulus) tend to exhibit ductile failure. In end-use applications where load-bearing capacity is required, rigid materials may be chosen but at the expense of inviting brittle failure. In some end-use applications, brittle failure is not desired. Furthermore, in some end-use applications, biodegradable and/or bioabsorbable materials are desired. Biodegradable and/or bioabsorbable materials typically are not sought for end-use applications where load-bearing capacity is required because they tend to lack the requisite rigidity. With respect to biodegradability and/or bioabsorbability, some end-use applications seek materials with an adequate rate of material degradation that does not result in compromising load-bearing properties before the useful life of the material has ended. Therefore, in end-use applications where rigidity is required for load-bearing purposes, ductile failure is a preferable failure mode, biodegradability and/or bioabsorbability is required, and a particular rate of material degradation is sought, no single material can bridge the gap between all of the aforementioned end-use requirements. This problem is communicated hereunder through the exemplary lens of composite implants for medical use.

In the event of bone fractures and certain medical conditions (e.g., osteoporosis), there are several conventional tools to support the bone during healing and/or fortify/augment a bone so it can withstand forces exerted during normal physical activity. External stabilizers (e.g., plaster casts, braces, etc.) tend to interfere with a patient's normal daily activities. In the field of veterinary medicine, some modes of fractures are difficult to apply external stabilizers to. Furthermore, shortly after application of the external stabilizer, the patient's intervening soft tissue begins to atrophy through disuse, thereby requiring further rehabilitation for the patient. Internal stabilizers (e.g., screws, bone plates, intramedullary nails, etc.) provide a more effective stabilization of the fracture than external stabilizers since they are able to directly interface with the bone. However, installing these internal stabilizers requires an invasive surgical procedure (i.e., a relatively large incision and displacement of tissue/organs/bone) and sometimes requires an additional procedure to remove the stabilizer.

Internal stabilizers (“orthopedic implants”) are typically fabricated from metals, polymers, and degradable biocomposites. With metal implants, removal surgery is often required or else they become permanent foreign objects in the body. Furthermore, pain is caused to the patient—with the intensity being dependent on where the implant is located. With polymeric implants, load bearing strength is lesser than metal implants and removal surgery is still required. With biocomposites, materials are substantially limited. As a result, it is difficult to achieve mechanical properties of the composite that are many times stronger than the loads expected during use. If a biocomposite were to fail, brittle failure is very traumatic to the patient and could injure the patient. Thus, it is important for the implant to exhibit ductile failure, which may still cause some trauma but would be far more preferable than brittle failure.

Biocomposite hardware (e.g., pins, plates, screws, etc.), while absorbed into the body over time and not needing to be removed, tend to be brittle and do not handle load bearing as well as metal implants. Brittleness may be attributed to the properties of biodegradable polymer and low aspect ratio fillers that make up biocomposite pins. This often results in breakage during the insertion procedure, adding cost and complexity to the procedure as well as trauma to the patient. In some circumstances, bone cements (e.g., polymer-based cements, calcium salt-based cements) are injected into the interior of the bone in an attempt to stabilize the bone. However, while these bone cements are typically capable of withstanding significant compressive loading, they are also extremely brittle and typically cannot withstand significant tensile loading. This limits their application in instances where the loading on the bone may include a tensile component, which is particularly seen in long bones (e.g., tibia).

The removable implants discussed above do not adequately meet the mechanical property needs to enable patients to perform normal physical activity without restriction. To do so, the materials require low density, stiffness, strength, and fracture resistance. However, conventional materials that achieve these mechanical properties are brittle. Thus, it can be appreciated that different materials and physical construction of implants pose trade-offs with respect to suitability for prolonged presence in the body and mechanical properties. Thus, it will be seen that a new approach is needed for designing and making implants with enhanced properties to meet the mechanical properties required for orthopedic implants.

In addition to mechanical properties, control of degradation rate is critical to the adequate performance of a degradable implant. If the implant degrades too rapidly, the implant may not have the strength required to provide structural support. If the implant degrades too slowly, a lag in degradation may result in negative changes to the environment in which the implant resides. There are three main stages of degradation which should be accounted for when designing a degradable implant. The initial degradation upon implantation drives the initial release of the implant's material into the body. Once the initial portion of the degradation profile has occurred, creating a steady state at which the degradation occurs over time is necessary. During steady state degradation, the transfer of the properties of the implant back to the healing bone occurs. This degradation rate needs to be tuned for the specific application. The last step of degradation is the final transfer from implant to the healed bone. Here it is important to design the implant such that the material is completely absorbed by the body. Ultimately the implant must be strong enough for the length of healthy process and then disappear. Conventional degradable implants struggle to cover all three stages of degradation, especially creating an implant that maintains the strength and/or transfers the strength back to the bone over the duration of the healing time.

It would be desirable to provide an implant that is bioabsorbable. It would be desirable to provide an implant that can withstand mechanical loads typically exerted through normal daily activity. It would be desirable to provide an implant that exhibits a ductile failure mode. It would be desirable to provide an implant with a tailored degradation. It would be desirable to provide an implant that can be formed into various types of hardware (e.g., pins, screws, plates, etc.). It would be desirable to provide an implant that is constructed with materials and processes that are commercially scalable.

### SUMMARY

The teachings herein relate to composite materials, to unique materials which may be employed in a composite material, and to methods for producing the composite materials or a component of the composite material. Due to unique properties of the materials, unique combinations of materials, and unique constructions of the composite materials, it is now possible to solve a variety of problems, particularly where biodegradability and/or bioabsorbability of the composite or one or more components of the composite is desired. These teachings find applicability in both medical applications and non-medical applications.

In one aspect, the teachings herein relate to coated fibers which may be used in a composite material. The coated fiber includes a plurality of aligned inorganic filaments and a coating of a polymerizable material or a polymeric material. The coated fiber typically has a substantially uniform profile along its length and are covered by the coating over at least a portion of their surface. The structure of the fiber and the materials of the filament and the coating are selected for providing good mechanical properties and/or flexibility. For example, the coated fiber may be characterized by one or more of the following characteristics: the coated fiber has a tensile modulus of about 10 GPa or more (preferably about 25 GPa or more, and more preferably about 40 GPa or more), as measured according to ASTM D638-14, using a fiber shaped specimen; or the coated fiber has a flexural modulus of about 5 GPa or more (preferably about 10 GPa or more, about 15 GPa or more, about 20 GPa or more, about 25 GPa or more, about 30 GPa or more, about 35 GPa or more, or about 40 GPa or more), as measured according to ASTM D 790-17; or the coated fiber has a bending radius of about 10 cm or less, as measured according to ASTM E290-14 (preferably where the coated fiber is bent until the legs contact). Preferably the inorganic filaments are preferably formed of an inorganic material having a specific gravity of about 2.80 or less, preferably about 2.65 or less, and most preferably about 2.50 or less. The inorganic material may be a metallic material or a non-metallic material. Preferably, the inorganic material is a non-metallic material. Preferably, some or all of the materials of the coated fiber are biodegradable and/or bioabsorbable.

In another aspect, the teachings herein relate to a fibrous bundle. The fibrous bundle may include two or more coated fibers which are in a polymeric matrix or may include two or more fibers having inorganic filaments which are together coated or impregnated by a polymeric material. The fibrous bundle has a high concentration of filaments which is typically about 55 volume percent or more, and the fibers are aligned in an axial direction, and the fibrous bundle has a substantially uniform profile along its length in the axial direction. The polymeric matrix may include or consist of the polymeric material that coats or impregnates the filaments or may include a different polymeric material. The structure of the fibrous bundle, the fiber and the materials of the filament, the polymeric matrix, and any polymeric coating are selected for providing good mechanical properties and/or flexibility. For example, the fibrous bundle may be characterized by one or more of the following characteristics: the fibrous bundle has a tensile modulus of about 10 GPa or more (preferably about 25 GPa or more, and more preferably about 40 GPa or more), as measured according to ASTM D638-14, using a fiber shaped specimen; or the fibrous bundle has a flexural modulus of about stiffness of about 5 GPa or more, as measured according to ASTM D 790-17; or the fibrous bundle has a bending radius of about 10 cm or less, as measured according to ASTM E290-14 (preferably where the fibrous bundle is bent until the legs contact). Preferably the inorganic filaments are formed of an inorganic material having a specific gravity of about 2.80 or less, preferably about 2.65 or less, and most preferably about 2.50 or less. The inorganic material may be a metallic material or a non-metallic material. Preferably, the inorganic material is a non-metallic material. Some or all of the materials of the fibrous bundle are preferably biodegradable and/or bioabsorbable. The fibrous bundle preferably includes a fiber having a twist in a first direction and an adjacent fiber having a twist in a reverse direction. Preferably the twists of the adjacent fibers provide a channel for receiving a liquid, such as via a wicking process.

In another aspect, the teachings herein relate to a fibrous composite including a plurality of fibrous bundles. The fibrous bundles are attached or connected by a plurality of bias fiber elements that wrap, weave together, braid, interlace, or interlock the fibrous bundles. In the fibrous composite, the fibrous bundles are aligned in an axial direction. Preferably, the bias fiber elements are angled relative to the axial direction. Preferably, the materials of the fibrous bundle and the bias fiber elements, and the attachment or connection allows for cooperative movement of the fibrous bundles or their fibers, so that the fibrous composite is ductile when deformed in compression, tension, flexion, bending, or any combination thereof. This ductility is maintained even when the concentration of fibers in the fibrous composite is generally high.

In yet another aspect, the teachings herein relate to a composite element having a core region including one or more of the fibrous composites which are at least partially covered by a second region including a polymeric covering. The composite element preferably includes one or more regions having a high concentration of inorganic filaments (including or consisting of the composite element(s)) and one or more regions which have a low concentration of filaments or which is free of inorganic filaments (including or consisting of the polymeric covering). Some or all of the materials of the composite element preferably are biodegradable and/or bioabsorbable. The composite element preferably has a generally uniform cross-sectional profile along a length of the composite element.

Another aspect of the teachings herein relates to a composite element comprising a core region including a plurality of fibrous bundles which are bound and/or interlocked by bias fiber elements wherein the core region includes a polymerizable material or a polymeric material, wherein the composite element has a second region including a polymeric covering formed of a polymerizable material or a polymeric material.

In another aspect, the teachings herein relate to a part including, or consisting of the composite element. For example, the part may have a core including one or more of the composite elements and a covering. By way of example, the part may be an implant, such as a screw or pin.

In another aspect, the teachings herein relate to a kit for forming a part, such as an implant, wherein the kit includes a plurality of composite elements and a polymerizable resin for impregnating a group of the composite elements. If the part needs to be contained, the kit may include a container, such as a containment bag. By way of example, for an implantable splint, the kit may include a containment bag for inserting into a medullary canal or other structure; a plurality of the composite elements for packing into the containment bag, and a polymerizable resin for inserting into the bag after the cylinders are introduced.

In another aspect, the teachings herein relate to the use of a kit for forming a part.

Another aspect of the teachings relates to the scaling of a component as described herein to provide a larger component including a plurality of the smaller components. For example, a scaled (i.e., larger) composite element comprising two or more smaller composite elements, preferably wherein the composite elements are interlaced or interlocked or bounded by bias fiber elements, such as in a braiding.

### BRIEF DESCRIPTION OF THE DRAWINGS

FIGS. 1-9 is a perspective view of a bone and illustrates a method for treating a bone fracture.

FIG. 10 illustrates a reinforcement element.

FIG. 11 illustrates the reinforcement element illustrated in FIG. 10.

FIG. 12 and FIG. 13 illustrate the reinforcement element illustrated in FIG. 10.

FIG. 14 illustrates a reinforcement element.

FIG. 15 illustrates the reinforcing element illustrated in FIG. 14, along the line A-A.

FIG. 16 illustrates a reinforcing element.

FIG. 17 illustrates the reinforcing element shown in FIG. 16.

FIG. 18 illustrates a cross-section of a fibrous bundle.

FIG. 19 illustrates a composite implant.

FIG. 20 illustrates the composite implant illustrated in FIG. 19, along line B-B.

FIG. 21 illustrates a cross-section of a composite implant.

FIG. 22 illustrates a cross-section of a composite implant.

FIG. 23 illustrates cross-sections of two composite implants.

FIG. 24 illustrates two configurations of reinforcement elements.

FIG. 25 illustrates a composite implant comprising reinforcing elements.

FIG. 26 illustrates cross-sections of composite implants.

FIG. 27 illustrate cross-sections of reinforcement elements.

FIG. 28 illustrate cross-sections of reinforcement elements.

FIG. 29 illustrates a cross-section of a composite implant.

FIG. 30 illustrates a segment W of the composite implant, illustrated in FIG. 29.

FIG. 31 illustrates a cross-section of a composite implant.

FIG. 32 illustrates segment X of the composite implant illustrated in FIG. 31.

FIG. 33 illustrates segment X of the composite implant illustrated in FIG. 32, according to another aspect of the present disclosure.

FIGS. 34A-34C illustrate composite implants in the form of a screw.

FIG. 35 is a perspective view and a cross-sectional view of a composite implant.

FIG. 36 is a perspective view and a cross-sectional view of a composite implant.

FIG. 37 illustrates a flowchart of a method according to the present disclosure.

FIG. 38 illustrates a screw.

FIG. 39 is a graph corresponding to the Examples.

FIG. 40 is a graph corresponding to the Examples.

FIG. 41 is a graph corresponding to the Examples.

FIG. 42 is a graph corresponding to the Examples.

FIG. 43 is a graph corresponding to the Examples.

FIG. 44 is a graph corresponding to the Examples.

FIG. 45 is a graph corresponding to the Examples.

FIG. 46 is a graph corresponding to the Examples.

FIG. 47 is a graph corresponding to the Examples.

FIG. 48 is a graph corresponding to the Examples.

FIG. 49 is a graph corresponding to the Examples.

FIG. 50 is a graph corresponding to the Examples.

FIG. 51 is a graph corresponding to the Examples.

### DETAILED DESCRIPTION

### A. Introduction

Conventional materials are subject to a number of trade-offs with respect to their properties. Rigid materials (e.g., those having a relatively high modulus, whether compressive, flexural, tensile and/or torsional) tend to exhibit brittle failure. In other words, they tend not to elongate measurably in plastic deformation before rupture. Relatively elastic materials (e.g., those having a relatively low modulus, whether compressive, flexural, tensile and/or torsional lower modulus) tend to exhibit ductile failure. In other words, they tend to elongate in plastic deformation before rupture. In end-use applications where load-bearing capacity is required, rigid materials may be chosen but at the expense of inviting a possible brittle failure. In some end-use applications, brittle failure is not desired. Furthermore, in some end-use applications, biodegradable and/or bioabsorbable materials are desired. Biodegradable and/or bioabsorbable materials typically are not sought for end-use applications where load-bearing capacity is required because they tend to lack the requisite rigidity. It has proven difficult to control biodegradability and/or bioabsorbability with many load bearing structures because such structures often lend themselves to potential localized degradation that may result in fragmentation. It is also a possibility that localized regions of degradation result in possible undesired acid buildup (e.g., within a body of a live being). Further, with respect to biodegradability and/or bioabsorbability, some end-use applications seek materials with an adequate rate of material degradation that does not result in compromising load-bearing properties before the useful life of the material has ended. Therefore, in end-use applications where rigidity is required for load-bearing purposes, ductile failure is a preferable failure mode. Biodegradability and/or bioabsorbability may be required, with a particular rate of material degradation is sought. Until the present teachings, composite materials have been unable generally to bridge the gap between all of the aforementioned end-use requirements, and to simultaneously address each of the various competing concerns. Some composite materials, while able to bridge the gap by providing some of the aforementioned end-use requirements fall short in providing all simultaneously. For example, some composites may offer rigidity and biodegradability but also exhibit brittle failure, which may be undesirable. Thus, the present teachings provide for a combination of materials (i.e., composite) combined and constructed in novel configurations that provide rigidity, ductile failure, biodegradability, and slow rate of degradation that have not been achieved by conventional composites. As described herein, the composite materials of the present disclosure are constructed in a novel manner to provide rigidity, ductile failure, biodegradability, and a slow rate of degradation (of the composite or any of its constituent elements). When employed as an implant, the composite materials of the present disclosure are also able to provide an attractive structure to foster implant compatibility with adjoining tissue and/or bone structures. For instance, as will be addressed, materials of the teachings can be employed to achieve an interconnected network of bone and/or tissue with structure of an implant. Such structure may change over time as the implant degrades. It will also be seen that the present teachings address difficulties in the art in terms of use of individual elements of resulting composites. That is, by way of example, it is often necessary for a surgeon to locate an implant or a component of an implant within a small confines of a body, which requires the component to be readily flexed and manipulated within the confines. The present teachings illustrate examples of components that achieve this result. Moreover, it will be seen that the teachings address a rapid approach to repairing a bone fracture. It will also be seen that the teachings find application outside of the field of orthopedic implants.

The present teachings pertain generally to new materials, combinations of materials and/or systems of materials. The teachings in general may find particular suitability in the design, manufacture and/or use of composite materials. For example, the teachings in general may find particular suitability in the design, manufacture and/or use of composite materials having one or more filaments and/or polymeric material forming one or more reinforcement elements. Such one or more filaments may be in contact with another material (e.g., by over at least a portion of their length) and/or dispersed in a matrix.

In accordance with a general aspect of the teachings, applicable to all embodiments (unless otherwise expressly stated), some or all of the one or more filaments and/or polymeric material may be biodegradable and/or bioabsorbable. For example, some or all of the one or more filaments and/or polymeric material may be bioabsorbable within a live being (e.g., a human or animal), so that they may be implanted into such live being, and following implantation they will be non-toxic to the live being, will not be rejected by the live being, and need not thereafter be removed from the live being.

A composite may be realized by the arranging one or more filaments and/or polymeric material to be in contact with one another in order to realize a desired structure. In general, the composites of the teachings herein may find beneficial application for non-load-bearing applications. However, among various benefits of the teachings in general is that composites in accordance with the teachings can be readily designed, made and/or used for load bearing applications. By way of illustration, composites of the present teachings may find application as orthopedic devices. Composites of the present teachings may find application as orthopedic devices as implantable devices that are implantable and/or implanted within a live being. Composites of the present teachings may find application as orthopedic devices as implantable devices that are implantable and/or implanted within a live being to repair a fracture of a bone (e.g., a partial and/or complete fracture of a bone). Composites of the present teachings may find application as orthopedic devices as implantable devices that are implantable and/or implanted within a live being to repair a fracture of a bone such as a transverse fracture, a spiral fracture, a comminuted fracture, an impacted fracture, a segmented fracture, a greenstick fracture, an oblique fracture, a stress fracture, a compression fracture, and/or an avulsion fracture.

The teachings herein generally contemplate a structure that includes a repaired bone fracture site that includes a first bone portion, a second bone portion and a composite (e.g., a composite implant device of the present teachings) connecting the first bone portion and the second bone portion. It is possible, for example, to locate a composite implant in a gap between a first bone portion and a second bone portion that has arisen from a fracture. A repaired bone fracture site of the present teachings may include a first bone portion, a second bone portion and a composite (e.g., an composite implant device of the present teachings) connecting the first bone portion and the second bone portion, wherein after a period of at least 1 week following implantation, a portion of the composite will have eroded and become absorbed. It is anticipated that following a period of time after implantation into a live being, implants (e.g., orthopedic or other implanted devices) will erode and the structure as implanted will cease to exist. Such period of time may be at least 1 week, 4 weeks, 8 weeks, 12 weeks, 18 weeks, 24 weeks, 36 weeks, 48 weeks or longer. Within and/or adjoining the volume originally occupied by the implant (e.g., the region of a treatment site), there will be an interpenetrating network of bone, tissue, and/or other bio-matter. The bio-matter may include materials from which filaments and/or polymeric material are fabricated according to the present teachings. These aspects of the teachings are useful for any bones within a human or animal skeletal structure.

Among various benefits of the present teachings is the possibility for composites that exhibit attractive load-bearing characteristics. This makes the materials of the teachings attractive for any of a variety of different applications, as a medical device or some other device (e.g., a device used for non-medical applications, such as a fastener). Within the medical device field, use as an implanted device is contemplated. Such implant device may be as a pin, a plate, a brace, a splint, a stent, a valve, a dental implant (e.g., one or more of an endosteal, subperiosteal, or zygomatic implant). As an orthopedic implant, composites of the present teachings find applicability for the repair of bones (e.g., within a human or animal, any cranial bones, jaw bones, spinal bones, leg bones, arm bones or otherwise). Application in the repair of load-bearing bones is a particularly attractive aspect of the teachings. Thus, for example, leg bones (e.g., a femur and/or a fibula) may be repaired to include an implant of the present teachings connecting a first bone portion with a second bone portion, as described previously. As additional examples, without limitation, an implant of the present teachings may be employed for connecting a first bone portion with a second bone portion wherein the bone portions are a part (without limitation) of a clavicle, a humerus, a radius, a tibia, an ankle, a hand, a foot, a cranial bone, a jaw bone, a cranial maxilla facial bone, ribs, or otherwise.

Among the capabilities of materials, material combinations and material systems in accordance with the present teachings are the possibility of realizing composites that exhibit attractive mechanical characteristics for load-bearing applications. For example, among possible technical benefits is the ability to realize a composite material, a structure made therefrom, or both that exhibits (i) a flexural modulus of at least 10, at least 15, or even at least 20 GPa; (ii) a strain to failure ratio of at least 0.02, at least 0.05, or even at least 0.10; (iii) or both (i) and (ii).

Among many benefits possible from the present teachings are the attractive capabilities of materials, material combinations and material systems to the in vivo fabrication of implants. In one aspect of the teachings, it is contemplated that kits may be provided to a user. A kit may include a plurality of implant precursor elements for introduction and assembly, at least partially or entirely, within a live being. The implant precursor elements may include one or more filaments, filament bundles, polymeric materials (e.g., in the form of a reactant, a pre-polymer, or otherwise), reinforcement elements, or any combination thereof. The implant precursor elements may be at least partially assembled outside a live being (i.e., ex vivo) and thereafter introduced within a live being. For example, an assembly of filaments, polymeric material, reinforcement elements, or any combination thereof may be assembled ex vivo and then introduced into a live being. Thereafter, one or more polymeric materials may be introduced into the live being to contact the assembly. The one or more polymeric materials after introduction may be controlled within the live being so a temperature does not exceed 44 C. Conditions within the live being may be controlled so a reaction or other transformation occurs for realizing a stabilized structure (e.g., by solidifying or hardening, either with or without an accompanying chemical reaction) that includes a polymeric-containing matrix at least partially contacting or at least partially surrounding at least a portion, if not all, of the filaments, polymeric material, reinforcement elements, or any combination thereof that had been introduced into the live being.

As the teachings herein will discuss, filaments, polymeric materials, reinforcement elements, and/or assemblies thereof may be provided to a user in a kit or otherwise in a size and shape that afford ease of forming an assembly ex vivo followed by insertion within relatively small confines within a fracture site of a live being. By way of illustration, filaments, polymeric materials, reinforcement elements, and/or assemblies thereof may be defined in size and shape to enable a resulting assembly to be introduced through a relatively small incision opening (e.g., less than about 20 centimeters (cm), less than about 10 cm, less than about 6 cm, less than about 4 cm, or even less than about 1 cm). Filaments, polymeric materials, reinforcement elements, and/or assemblies thereof may be defined in size and shape to enable a resulting assembly to be manipulated by a user (e.g., a surgeon) in vivo around a surface having a relatively small radius of curvature of less than (e.g., less than about 10 centimeters (cm), less than about 6 cm, less than about 4 cm, or even less than about 1 cm). Filaments, fibers, and/or fiber bundles may be defined in size and shape to enable a resulting assembly to be manipulated by a user (e.g., a surgeon) in vivo within a fracture site having a gap (between a first bone portion and a second bone portion) of less than about 10 centimeters (cm), less than about 6 cm, less than about 4 cm, or even less than about 1 cm). Accordingly, from the above (and as will be discussed in further detail herein), the present teachings lends itself well to minimally invasive surgical techniques and attendant benefits of such techniques.

Among the attractive capabilities of materials, material combinations and material systems in accordance with the present teachings are the ability to implant them within a live being so that they become a composite orthopedic implant in vivo, only after introduction into a live being. In view of the materials, material combinations and material systems in accordance with the present teachings, comparatively fast orthopedic implants can be realized from a point of time from when an incision is made. It is possible, for example, from a time when any reactants, precursor materials, or other polymeric-containing or polymeric forming ingredients are activated following introduction into the living being until the material solidifies, hardens or otherwise rigidifies, a time elapses that is less than about 120 minutes, less than about 100 minutes, less than about 80 minutes, less than about 60 minutes, less than about 40 minutes, less than about 30 minutes, less than about twenty minutes or less than about 10 minutes may elapse. (need to reconcile this with gel times and cure times). In this manner, procedure times may be shortened, and a user (e.g., a surgeon) may be able to direct attention much more quickly to demands of other patients.

Within an illustrative application of the present teachings (namely, an orthopedic implant and method of making the implant for repairing a fracture), a unique property profile can be achieved that affords attractive mechanical properties for facilitating healing of a bone fracture (e.g., a stiffness characterized by a flexural modulus of about 10 GPa or more (preferably 15 GPa or more, and more preferably about 20 GPa or more) and ductility characterized by a strain at failure in torsion and/or tension of about 0.02 or more, preferably about 0.05 or more, and more preferably about 0.10 or more)) for a period of at least one week, two weeks, three weeks, or four weeks from time of implant formation in vivo until the implant starts to degrade and absorb.

Examples within the teachings will be described herein in which it will be seen that resulting implants can be characterized by an attractive relatively high level of surface area per unit volume occupied by the “footprint” of the implant. This may be achieved in any of a number of ways, including the formation of channels, divots, holes, surface roughening, or the like. It will also be seen herein that after a period of time following formation of an implant in vivo, an interpenetrating network will be realized as between at least a portion of the implant and bone or tissue that has grown within channels, divots, holes, and/or surface voids defining its roughness. The material of the implant and that of the bone or tissue will include plural protuberances resulting from the newly formed bone or tissue.

Following a period of 1 week from the time of implant formation, the bone in the region of an edge of bone portion that contacted the implant at time of formation of the implant (e.g., within about 7 cm, 5 cm, 3 cm, or 1 cm) may show an enrichment of certain chemical elements (e.g., sodium, calcium, phosphorus or other element) as compared with bone located remotely (e.g., greater than about 10 cm, 15 cm or more).

As will be discussed, among various unique features attainable with the present teachings is a controlled failure mode in the event that a composite (e.g., an implant composite) is subjected to a load that exceeds the ultimate strength exhibited by one or more of the constituent materials of the composite. For example, it is possible that upon being subject to a load that exceeds the maximum compressive strength of a polymeric-containing polymeric material within a composite of the present teachings, the polymeric-containing polymeric material will form a crack that propagates until it reaches a fiber or fiber bundle, at which energy from the load will be dissipated by fiber or fiber bundle due at least in part from the travel of adjoining filaments within a fiber relative to each other, the travel of adjoining fibers within a fiber bundle relative to each other, the travel of adjoining fiber bundles relative to each other, or any combination thereof. As a result of such relative travel of filaments, fibers and/or fiber bundles, it is possible that the composite will exhibit a ductility that exceeds the ductility of individual components.

### B. Composite Article, Constituent Elements Thereof, and Materials Forming the Same

The present invention uses a hierarchal construction of precursor elements to achieve a commercially scalable composite. The teachings are illustrated with reference to a composite implant. However, the teachings are applicable generally to other composite articles. Accordingly, the following teachings should be regarded as generalized teachings for composite articles and not limited to composite implant.

The present disclosure provides for a composite article (e.g., composite implant) that is simultaneously rigid, exhibits ductile failure, is biodegradable and/or bioabsorbable, degrades at a controlled rate, and is elastic.

The composite implant may be used for medical applications, non-medical applications, or both. The medical applications may include human medicine and veterinary medicine. The composite implant may be employed for treating bone fractures, fortifying bone, augmenting bone, or any combination thereof. The composite implant may do so while exerting minimum inconvenience to a patient. The composite implant may be placed in a patient via a minimally invasive procedure. For example, the composite implant may be placed into a patient via laparoscopic procedures. The composite implant may be in any suitable form typically utilized for orthopedic implants. The forms may include, but not limited to, screws, pins, plates, braces, splints, anchors, the like, or any combination thereof. The pins may have a cross-sectional shape including circular, ovoid, triangular, quadrilateral, pentagonal, hexagonal, octagonal, or any combination thereof. The composite implant may function as a splint, whereby the composite implant carries mechanical stress created during patient activity. The composite implant may be located within an intramedullary canal of a bone, within an opening formed into a bone (e.g., by surgeons), or any other suitable opening in a bone.

As seen from the above, as a result of structures disclosed herein, the teachings have broader application than only implants. For example, pins, screws, plates or other articles can be made in accordance with the present teachings and used (alone or in combination) for other applications (e.g., non-implant or non-medical applications). The composite article (e.g., composite implant) may be an elongate member (e.g., screw, pin, etc.). The elongate member may have a length, width, diameter, cross-sectional length, cross-sectional width, or any combination thereof. The elongate member may have an aspect ratio. The aspect ratio may be defined by a ratio of length to width. The aspect ratio may be associated with a length and width, a cross-sectional length and cross-sectional width, or both.

The composite article (e.g., composite implant) may comprise one or more containment bags, filaments, fibers, fibrous bundles, fibrous composites, polymeric materials, fillers, or any combination thereof. These may be referred to alternatively as “precursor elements”. When employed for making an composite element, each of the precursor elements may construct a composite implant before introduction into a patient. The composite implant may be at least partially pre-cured before introduction into a patient. Each of the precursor elements may be sequentially added into a patient and a composite implant may be constructed in situ. To this end, the precursor elements may have a size and flexibility to be delivered through a catheter (e.g., having an inner diameter of at least at about 1 millimeters (mm), at least about two mm, less than about 15 mm, less than about 10 mm, such as from about 2 mm to about 9 mm). Each of the precursor elements may be hierarchically structured so the composite implant may be custom tailored for different treatment situations. In this manner, the composite implant can be fabricated to suit different lengths, widths, cross-sectional dimensions, or any combination thereof. As referred to herein, hierarchically structured may mean arranging smaller elements together to form even larger elements, optionally arranging said larger elements together to form even larger elements, and so on.

Each of the precursor elements may be selected to provide in the resulting composite (e.g., composite implant) particular mechanical properties according to the need of individual patients. The mechanical properties may include tensile strength, compressive strength, flexural strength, torsional strength, ductile failure mode, or any combination thereof.

The composite article (e.g., implant) may comprise one or more regions. The regions may include one or more cores, outer regions, or both. The regions may be constructed in a hierarchal fashion. The regions may be constructed in a modular fashion. The modularity of the regions may provide for a scalable manufacturing process for composite articles. The one or more regions may be formed from one or more precursor elements. Each of the one or more regions may provide the same or different mechanical properties (e.g., tensile strength, compressive strength, flexural strength, torsional strength) and/or degradation properties (e.g., degradation rate) to the composite article. The one or more regions may include one or more cores, outer regions, or both.

The one or more regions may include one or more cores. The cores may function to provide tensile strength, compressive strength, flexural strength, torsional strength, or any combination thereof to the composite article. Each of the cores may have the same or different mechanical properties and/or degradation properties. The composite article may comprise 1 or more, 2 or more, 3 or more, or even 4 or more cores. The composite article may comprise 10 or less, 9 or less, 8 or less, 7 or less, or even 6 or less cores. Two or more cores may be concentrically located one within another.

Divisions between cores and/or between cores and outer regions may be observed when viewing a transverse cross-section of a composite article. The cores may have a thickness of 0.1 mm or more, 1 mm or more, 5 mm or more, 10 mm or more, or even 20 mm or more. The cores may have a thickness of 1,000 mm or less, 800 mm or less, 300 mm or less, 100 mm or less, or even 50 mm or less. The thickness may be measured along a line extending between a central axis of the composite article to an outer edge of a composite article. The thickness may be observed when viewing a transverse cross-section of a composite article.

The core may comprise one or more filaments, fibers, fibrous bundles, fibrous composites, polymeric materials, fillers, or any combination thereof. The core may comprise 1 or more, 10 or more, 20 or more, or even 30 or more fibrous bundles, fibrous composites, or both. The core may comprise 100 or less, 90 or less, 80 or less, or even 70 or less fibrous bundles, fibrous composites, or both. The combinations of precursor elements fabricated from different materials and/or arranged in various geometric configurations may provide for properties of the composite article that may not be achieved with standard laminate techniques.

One or more cores may be fabricated from the same or different precursor elements than one or more other cores. For example, one core may include a first type of polymeric material and another core may include a second type of polymeric material. One or more cores may include a number of precursor elements, orientation of precursor elements, format of precursor elements, fiber volume, or any combination thereof that is the same as or different from one or more other cores. An assembly may include plural cores having similar structures, or plural cores having dissimilar structures. For example, one core may comprise one or a plurality of fibers and/or fibrous bundles oriented at a first angle (e.g., about +30°, +45°, or +60°) angle, with respect to a longitudinal axis of a composite article, and another core may comprise one or a plurality of fibers and/or fibrous bundles oriented at a second angle (e.g., about −30°, −45°, or)−60° angle, with respect to a longitudinal axis of a composite article. Angular orientations may differ from those identified in the above illustrations)

One or more fibrous bundles may bind, interlock, interlace, or any combination thereof one or more other fibrous bundles in a core. Forces translating across a thickness of a core may be displaced by fibrous bundles that bind, interlock, interlace, or any combination thereof. Forces directed toward a distal portion of a core from a proximal portion of a core, or vice versa, may be translated by fibrous bundles that bind, interlock, interlace, or any combination thereof. Interlocking may provide for transmission of forces through the thickness of the composite without the typical delamination between layers seen with laminate composites.

One or more fibrous bundles may interlock two or more cores. One or more fibrous bundles may interlock one or more cores with one or more outer regions. One or more cores may be bound by one or more fibrous bundles.

Arrangements of cores, fibers and fibrous bundles may be employed for realizing in a resulting composite article (e.g., a composite implant) a load bearing structure, that exhibits attractive rigidity and ductility characteristics (as described elsewhere herein), biodegrability (as described elsewhere herein), or a combination of each.

After placement of the precursor elements, the cores may be further worked via coating, forming, molding, over molding, machining, or any combination thereof to create a composite article (e.g., implant). Precursor elements within the same core may each contribute unique attributes to the core as a whole. The fibrous bundles may be ordered or not ordered to provide the composite article (e.g., implant) with unique attributes. As referred to herein, ordered mean generally uniformly aligned (e.g, an average alignment that deviates from an axis by less than 10°). For example, the fibrous bundles may be arranged in layers and fibrous bundles of each layer may be axially aligned, have consistent offset alignment, or both. “Not ordered” refer to packing in a random spatial distribution. The use of ordered and not ordered fibrous bundles may be selected and used in combination to achieve a desire may cause different wetting out of material throughout the fibrous bundles, during a later step of infiltrating the bundles with a polymeric-containing material.

The one or more regions may include one or more outer regions, which may be discrete regions that are distinguishable from each other on the basis of a defined border, such as a physical interface therebetween. The outer regions which are located toward an external surface of a fibrous bundle may function as a permeable barrier to control the ingress of fluids into the core, control the degradation rate of the composite article, compatibilize the composite article (e.g., implant) with a surrounding environment, deliver one or more functional agents (e.g., a therapeutic medicament, in the case of a composite implant) into a surrounding environment, or any combination thereof. The composite article may comprise 1 or more, 2 or more, or even 3 or more outer regions. The composite article may comprise 8 or less, 7 or less, or even 6 or less outer regions. Each of the outer regions may provide a unique degradation rate. The outer regions may be applied to one or more cores via coating, molding, over molding, or any combination thereof to create a composite article. After application of the outer regions, the outer regions may be formed, machined, or both.

Divisions between outer regions and/or between cores and outer regions may be observed when viewing a transverse cross-section of a composite article. The outer regions may have a thickness (as measured of 0.01 mm or more, 0.1 mm or more, or even 1 mm or more. The outer regions may have a thickness of 1 cm or less, 8 mm or less, 5 mm or less, or even 2 mm or less. The thickness may be measured along a line extending between a central axis of the composite article to an outer edge of a composite article. The thickness may be observed when viewing a transverse cross-section of a composite article.

A ratio of reinforcement elements included in the cores and reinforcement elements included in the outer regions may be 1:0.01 or more, 1:0.1 or more, or even 1:0.5 or more. A ratio of reinforcement elements included in the cores and reinforcement elements included in the outer regions may be 1:2 or less, 1:1.5 or less, or even 1:1 or less.

The outer regions may be fabricated from one or more filaments, fibers, fibrous bundles, fibrous composites, polymeric materials, fillers, or any combination thereof. One or more outer regions may be fabricated from the same or different material than one or more other outer regions. For example, one outer region may include a first type of polymeric material and another outer region may include a second type of polymeric material. To control diffusion rates, the outer region may include a sizing agent applied thereto.

The composite article may include one or more surface features. The one or more surface features may arise from spatial arrangements of one or more fibrous bundles, outer regions, or both. The spatial arrangements of fibrous bundles may result in unique cross-sectional shapes of the composite article. The outer regions may be molded, over molded, formed, machined, or any combination thereof to create surface features.

The surface features may extend at least partially between opposing longitudinal ends of a composite article. The surface features may extend from a surface of a composite (e.g., a composite implant). The surface features may extend radially around a composite. The surface features may extend longitudinally along a composite. They may extend radially, helically, or otherwise about an axis of a composite.

The surface features may include facets, corners, lobes, barbs (e.g., to help secure to bone), knurls, protrusions, threads (e.g., screw threads), heads (e.g., screw head), tapered ends, or any combination thereof. The facets may include one or more edges of a cross-sectional shape extending between distal ends of a composite article. For example, a composite article with a hexagonal cross-sectional shape may include six facets. The facets may meet at corners. The corners may be sharp, tapered, or both. The composite article may include one or more lobes. The number of lobes may be determined by an arrangement of fibrous bundles in a core. The lobes may be fabricated by a mold, coating over a natural contour of precursor elements, or both. The lobes may include a radius. The lobes may function to minimize stress when a composite article is inserted into bone or other suitable substrate. The tapered ends may function to provide for easy insertion of a composite article into a structure. The tapered ends may function to provide for easy insertion of a composite article into bone or other suitable substrate. The tapered ends may be formed at opposing ends of a composite article (e.g., implant). The surface features may be fabricated with a twist around a longitudinal axis at a pitch of about 0 to 1 revolution per cm of axial length. The pitch may vary along any portion of a longitudinal axis of a composite article. The surface features may be aligned with and/or offset from fibrous bundles.

The composite article (e.g., composite implant) may comprise one or more cannulations. The cannulation may be a hollow shaft within a composite article. The cannulation may extend at least partially between and/or through distal ends of a composite article. The cannulation may extend longitudinally through the composite article, through the center of the composite article (e.g., implant), between two distal ends of the composite article (e.g, implant, or any combination thereof. The cannulation may have a cross-sectional shape including circular, ovoid, triangular, quadrilateral, pentagonal, hexagonal, octagonal, the like, or any combination thereof. The cannulation may be formed by locating a plurality of precursor elements around a removable mandrel. The cannulation may act as or it may define a driver socket or driver head to aid introduction of an article composite article into bone or other suitable substrate using a driver tool. The cannulation may extend at least partially through a screw head.

The composite article (e.g., composite) may include one or more apertures. The apertures may be through-holes. The apertures may extend only partially along a radius of the composite article. When not employed as through-holes the apertures may extend 5% or more, 10% or more, 20% or more, or even 30% or more the radius of the composite article. When not employed as through-holes the apertures may extend 100% or less, 90% or less, 80% or less, or even 70% or less the radius of the composite article (e.g, composite implant). The apertures may extend completely through the diameter of the composite article (e.g, composite implant).

The apertures may be located around a perimeter of the composite article (e.g., composite implant). The apertures may be located along a length of the composite article (e.g., composite implant). The holes may protrude inwardly toward a center axis of the composite article (e.g., composite implant). The apertures may be in longitudinal alignment. The apertures may be off-axis with respect to each other. The apertures may be elongated having a major axis and a minor axis (e.g., they may be oval or elliptical). The apertures may be circular, ovoid, the like, or any combination thereof.

The cannulation and/or apertures may occupy a volume of about 5% or more, 10% or more, or even 15% or more, with respect to the total volume of the composite article (e.g., composite implant). The cannulation and/or apertures may occupy a volume of about 40% or less, 30% or less, or even 20% or less, with respect to the total volume of the composite article (e.g., composite implant). The cannulation and/or apertures may add surface area to the composite article (e.g., composite implant).

In one aspect of the present disclosure, the composite article (e.g., composite implant) may include or constitute a screw. The screw may include threading (surface feature). The threading may have a thread angle, pitch, crest, root, major diameter minor diameter, or any combination thereof. The major diameter may be the cross-sectional diameter of the root. The minor diameter may be the cross-sectional diameter of the crest. The threading may have a pitch. In a single winding (pitch) there may be one or more apertures.

In constructing a screw (or other elongated article), one or more first fiber bundles may be built up around a removable mandrel, defining one or more cores; one or more second fiber bundles (reinforcement elements) may then be wound around the first fiber bundles, defining threads. The second fiber bundles may be wound helically around the first fiber bundles; then the mandrel may be removed, leaving behind a cannulation. Before or after removal of the mandrel, the first and second fiber bundles may be coated, defining an outer region.

There are many design trade-offs that are made to modulate the functions and properties of the composite. Functions and properties tuned in the composite of the present disclosure may include, but are not limited to one, two, three, four, or any combination of characteristics selected from tensile strength and/or stiffness, compressive strength and/or stiffness, flexural strength and/or stiffness, torsional strength and/or stiffness, sheer strength and/or stiffness, biodegradability and/or bioabsorbability, flexibility, ductility, strain to yield, thickness, density, volume, length, shape, bending, stretching, torsion, bending-stretching coupling, failure, fiber volume, elastic modulus, wetting out, toughness, responsiveness, fracture toughness, mechanical strength, brittle, viscosity, tensile strength, poly dispersion index, damping, fiber bundle angles, flexural modulus, % strain, torsion, bending, inherent viscosity, pore diameter, compression, tortuosity, modulus, glass transition temperature (Tg), number of layers, pull-out force, roughness, driving torque, moisture content, threading, and aspect ratios. For example, an article in accordance with the teachings may include an arrangement of fiber bundles configured to achieve (in a resulting composite) relatively higher compressive stiffness, flexural stiffness, torsional stiffness, and/or ductility as compared with the bulk properties of each individual material of the composite.

A composite material of the present teachings may have a strain to yield of less than 2%, 5%, 10%, 15%, or even 20% in bending. The composite may have a strain to failure of at least about 2% or more, 5% or more, 10% or more, 12% or more, 15% or more, or even 20% or more in bending. The composite may have a failure mode that is not catastrophic at about 2% or more, 5% or more, 10% or more, 12% or more, 15% or more, or even 20% or more strain in bending.

The composite material may have an elastic modulus that is at least about 8 GPa, 10 GPa, 12 GPa, 15 GPa, 20 GPa, or even 25 GPa in bending.

A composite material of the present teachings implant may have a strain to yield of at least about 2%, 3%, 5%, 15%, or even 20% in torsion. The material may exhibit at least about 2°, 5°, 10°, 15°, 20°, or even 25° of angular deflection at the yield point in torsion. A material may exhibit a strain to failure of at least 2%, 5%, 10%, 12%, 15%, or even 20% in torsion. The composite material may be designed to exhibit a failure mode that is ductile. For instance, it may exhibit at least 2%, 5%, 10%, 12%, 15%, or even 20% strain in torsion. The material may have at least 10°, 20°, 30°, 45°, 60°, 90°, or even 120° of angular deflection at the failure point in torsion. The material may have a failure mode is not catastrophic. For example, it may exhibit at least 10°, 20°, 30°, 45°, 60°, 90°, or even 120° of angular deflection in torsion.

The composite material may have an elastic modulus is at least 5 GPa, 8 GPa, 10 GPa, 12 GPa, 15 GPa, or even 20 GPa in torsion.

The material may have a strain to yield of at least 2%, 5%, 10%, 15%, or even 20% in compression. The material may have a failure mode that is not catastrophic. For example, it may exhibit at least 2%, 5%, 10%, 15%, or even 20% strain in compression. The material may have a strain to failure of at least 2%, 5%, 10%, 15%, or even 20% in compression. The composite material may have an elastic modulus of at least 10 GPa, 15 GPa, or even 20 GPa in compression. The material may have a tensile modulus of less than 60 GPa.

Composite materials of the present teachings may be characterized as exhibiting a stress strain curve (in response to tensile loading, compression loading or torsional loading) that includes a first portion from which the modulus can be determined. Following the first portion, and from a portion of the curve when yield starts to occur, the stress-strain curve includes a portion that exhibits a sawtooth pattern. That is, over there are a plurality (e.g., at least 2, 3, 4, 5, 6, 7 or more) fluctuations of stress over a range of strain values, prior to rupture. The fluctuations of stress over the range of strain values may may be attributable to relative translation that takes place as between two or more adjoining fiber bundles of the composite material. This may be particularly the case in instances when two or more of the fiber bundles (e.g., adjoining fiber bundles) include a bias reinforcement element winding about them.

The performance of a composite article may be improved by one or more of the following: modular construction of precursor elements, polymeric material filling interstitial spaces between precursor elements; interlocking precursor elements; polymer selection for polymeric material; incorporation of energy absorbing fillers; or any combination thereof.

A kit for preparing a composite part may include one or more containers for holding one or more components of a composite part to be made from the components. The container may contain a polymeric material (which may be provided in a liquid phase) and filament or fiber containing component such as a fibrous bundle, a fibrous composite, or a composite element. The container may be flexible or rigid. The container may be sufficiently flexible so that the container can be inserted into an opening and then expand so that a pressure is applied to a wall of the opening.

The container may be a may include one or more containment bags. The containment bag may function to protect the composite article from ingress of blood and/or other bodily fluids that might interfere with the deployment of reinforcing elements, deployment of polymeric material, solidification of polymeric material, accelerate degradation, or any combination thereof. The containment bag may function to constrain the flow of polymeric material while the polymeric material is in an injectable (flowable) state. The containment bag may have sufficient strength to allow the polymeric material to be injected into the containment bag under substantial pressure so as to ensure good interfacial contact between the polymeric material and one or more reinforcing elements, containment bag, bone, or any combination thereof. Pressure may be employed to minimize voids within the containment bag. The containment bag may be flexible.

The containment bag may be fabricated from a bioabsorbable polymer, fibers, or both. The bioabsorbable polymer may include polyurethane, polylactic acid, glycolic acid, copolymers thereof, or any combination thereof. The fibers may be woven, braided, knit, non-woven, the like, or any combination thereof. The fibers may form a mesh bag. Suitable fibers may include polylactic acid, polyglycolic acid, polydioxanone, copolymers thereof, bioabsorbable glass, soluble glass, metal, or any combination thereof. The mesh bag may be hydrophobic so as to minimize the ingress of bodily fluids into the containment bag. The mesh bag may have a limited porosity to allow some egress of polymeric material out of the containment bag (e.g., to osseointegrate with the surrounding bone).

The containment bag may have a porosity. The porosity may be varied across the extent of the containment bag so as to provide regions of greater or lesser porosity to the polymeric material, thus providing control of the ability of the polymeric material to infiltrate the surrounding bone. The containment bag may have an average pore size of about 0.0001 μm or more, 0.001 μm or more, 0.01 μm or more, or even 0.1 μm or more. The containment bag may have an average pore size of about 1000 μm or less, 100 μm or less, 10 μm or less, or even 1 μm or less. The containment bag may have a thickness (wall thickness) of about 0.01 mm or more 0.1 mm or more, or even 1 mm or more. The containment bag may have a thickness (wall thickness) of about 5 mm or less, 3 mm or less, or even 2 mm or less.

The composite may be fabricated from one or more filaments, fibers, fibrous bundles, fibrous composites, composite elements, or any combination thereof. A plurality of filaments may be substantially axially aligned, nested, and assembled into a fiber. Axially aligned may refer to alignment along the longitudinal axis of the filaments. Nested may refer to joining into one structure such that adjacent elements interface with each other. The fiber may be twisted, coated, uncoated, or any combination thereof. A plurality of fibers may be substantially axially aligned, nested, and assembled into a fibrous bundle. The fibrous bundle may be coated, uncoated, braided, unbraided, or any combination thereof. Two or more fibrous bundles may be joined together to form a fibrous composite. The fibrous composite may be formed by braiding, weaving, axially aligning, suspended in a polymeric material, or any combination thereof. The fibrous composite may be coated, impregnated, or both with polymeric material. The fibrous composite may have a form of a sheet, rod, tube, roll, tape, or any combination thereof. Two or more fibrous composites may be assembled together to form a composite element. The composite element may include a final composite element, a scalable composite element, or both. A final composite element may be employed as a composite article. For example, a final composite element may be employed as a composite article suitable for introduction into a body of a patient. A scalable composite element may be combined with one or more other scalable composite elements to form a final composite element. Fibers, fibrous bundles, fibrous composites, composite elements, or any combination thereof may be alternatively referred to as reinforcement elements.

The composite may be fabricated from a fiber comprising a plurality of filaments. The filaments may be assembled to construct fibers (coated or uncoated), fibrous bundles, fibrous composites, composite articles, or any combination thereof.

The filaments may be formed of an organic material, an inorganic material, or both. Suitable inorganic materials may have a specific gravity of about 2.80 or less, about 2.65 or less, about 2.50 or less, or about 2.45 or less. The specific gravity of the inorganic materials may be about 1.50 or more, about 1.70 or more, about 1.90 or more, or about 2.10 or more.

The filaments may be combined with polymeric material, filler, or both to construct one or more reinforcement elements. The filaments may have an aspect ratio (i.e., ratio of length to width) of from about 1:1 to about 1:10,000, about 1:10 to about 1:1,000, or even about 1:20 to about 1:100. The filaments may have an average diameter of about 0.1 μm to 1 mm, or even 3 μm to 50 μm.

The filament, fiber, or coated fiber may have a high modulus, a low elongation, or both, as measured according to ASTM D638. The filament, fiber or coated fiber may have a tensile modulus of about 0.08 GPa or more 0.1 GPa or more, 1 GPa or more, 10 GPa or more, 20 GPa or more, 30 GPa or more, 40 GPa or more, or even 50 GPa or more. The filament, fiber or coated fiber may have a tensile modulus of about 100 GPa or less, 90 GPa or less, 80 GPa or less, 70 GPa or less, or even 60 GPa or less. The filament, fiber, or coated fiber may have an elongation of about 2% or more, 10% or more, 30% or more, 70% or more or even 100% or more. The filament, fiber, or coated fiber may have an elongation of about 500% or less, 400% or less, 300% or less, or even 200% or less. The filament, fiber, or coated fiber may have a tensile strength of about 10 MPa or more, 20 MPa or more, 40 MPa or more, or even 60 MPa or more. The filament, fiber, or coated fiber may have a tensile strength of about 150 MPa or less, 130 MPa or less, 110 MPa or less, 90 MPa or less, or even 70 MPa or less.

The filaments may be fabricated from material that is biodegradable, bioabsorbable, non-biodegradable, non-bioabsorbable, water soluble, carbon neutral, or any combination thereof. Examples of suitable biodegradable and/or bioabsorbable materials may include but are not limited to one or any combination of those enumerated in U.S. Patent Publication No. 2019/0175734 A1, incorporated herein by reference in its entirety for all purposes (see, e.g., paragraph 31 and paragraph 137). Other examples of suitable biodegradable and/or bioabsorbable material may include but are not limited to one or any combination of glass compounds and/or ions enumerated in U.S. Patent Publication No. 2019/0269823 A1, incorporated herein by reference in its entirety for all purposes (see, e.g., paragraph 25 and paragraph 26). By way of further example but not limitation, suitable bio-neutral materials include natural polyesters and silks, polyvinyl alcohol, glass, ceramic, metal, and carbon fiber.

The glass compounds may comprise additional elements selected from the group consisting of Cu, Sr, Zn, Fe, Mn, Cr, V, Nb, Mo, W, Ba, Co, S, Al, Ti, Y, Mg, Si, F, Zn, Ni, or any combination thereof. In one aspect of the present disclosure, the glass compound may include borate-based glass material containing one or any combination of Ag2O (0-20%), Al2O3 (0-20%), B2O3 (0-85%), BaO (0-60%), CaO (0-50%), Ce (0-20%), Cl (0-30%), Co (0-20%), Cr (0-20%), CuO (0-20%), F (0-20%), Fe2O3 (0-40%), Ga2O3 (0-20%), K2O (0-35%), Li2O (0-35%), MgO (0-40%), MnO (0-20%), MoO (0-20%), Na2O (0-35%), NaF (0-20%), NH (0-30%), NO (0-50%), Nb (0-20%), P2O5 (0-20%), P2O4 (0-20%), P2O3 (0-20%), Rb2O (0-35%), S (0-20%), SiO2 (0-30%), SrO (0-45%), St (0-20%), TiO2 (0-20%), V (0-20%), W (0-20%), Y (0-20%), ZnO (0-20%), ZrO2 (0-20%) (all percentages are by weight, unless stated otherwise stated). In another aspect of the present disclosure, the glass compound may include silicate-based glass containing one or any combination of Ag2O (0-20%), Al2O3 (0-20%), B2O3 (0-40%), BaO (0-60%), CaO (0-50%), Ce (0-20%), Cl (0-30%), Co (0-20%), Cr (0-20%), CuO (0-20%), F (0-20%), Fe2O3 (0-40%), Ga2O3 (0-20%), K2O (0-35%), Li2O (0-35%), MgO (0-40%), MnO (0-20%), MoO (0-20%), Na2O (0-35%), NaF (0-20%), NH (0-30%), NO (0-50%), P2O5 (0-20%), P2O4 (0-20%), P2O3 (0-20%), Rb2O (0-35%), S (0-20%), SiO2 (15-95%), SrO (0-45%), St (0-20%), TiO2 (0-20%), V (0-20%), W (0-20%), Y (0-20%), ZnO (0-20%), ZrO2 (0-20%) (all percentages are by weight, unless stated otherwise stated). In another aspect of the present disclosure, the glass compound may include phosphate-based glass containing one or any combination of Ag2O (0-20%), Al2O3 (0-20%), B2O3 (0-40%), BaO (0-60%), CaO (0-50%), Ce (0-20%), Cl (0-30%), Co (0-20%), Cr (0-20%), CuO (0-20%), F (0-20%), Fe2O3 (0-40%), Ga2O3 (0-20%), K2O (0-35%), Li2O (0-35%), MgO (0-40%), MnO (0-20%), MoO (0-20%), Na2O (0-35%), NaF (0-20%), NH (0-40%), NO (0-50%), Nb (0-20%), P2O5 (0-70%), P2O4 (0-70%), P2O3 (0-70%), Rb2O (0-35%), S (0-20%), SiO2 (0-30%), SrO (0-45%), St (0-20%), TiO2 (0-20%), V (0-20%), W (0-20%), Y (0-20%), ZnO (0-20%), ZrO2 (0-20%) (all percentages are by weight, unless stated otherwise stated). Ion's utilized for ion exchange, discussed hereinafter, may include any one or combination of elements listed above. By way of example but not limitation, glass filaments may comprise a glass sheet, a rod, a sphere, a triangular prism, a rectangular prism or any three-dimensional shape with more than three sides.

The filaments may include a surface modification (“coating”, “treatment”). The surface modification may function to provide a functional surface and/or inner layers to the fiber for improved adhesion to polymeric material. The surface modification may alter chemical durability, mechanical properties, functionality (e.g., ion doping), or any combination thereof. The surface modification may functionalize the glass for a microenvironment in which the glass' use is intended. For example, the surface modification may functionalize the glass to be more bioactive (e.g., for stimulating osteogenesis or angiogenesis), impart bacterial and/or microbial control (e.g. antibacterial, antimicrobial, bacterial static, biocidal, or any combination thereof), create a gradient of and/or change in refractive index, or any combination thereof.

Some, or all of the filaments may include a surface modification. The surface modification may be applied to filaments before or after assembly into a fibrous bundle. The surface modification may penetrate to various depths. A glass filament, having a cross section (c), may be surface treated to a depth of c or less, 0.5c or less, or even 0.01c or less. Different surface modifications on the same glass may penetrate to the same or different depths. For example, a glass, having a cross section (c), may be subjected to a first surface modification to a depth of less than or equal to 0.25c and a second surface modification to a depth of less than or equal to 0.25c. Different surface modifications on the same glass may penetrate to a combined total depth of c. The depth of the surface modification may be about 1 μm or more, 3 μm or more, 10 μm or more, 30 μm or more, or even 60 μm or more. The depth of the surface modification may be about 400 μm, or less, 300 μm or less, 200 μm or less, 150 μm or less, 100 μm or less, or even 80 μm or less.

Examples of suitable surface modifications may include but are not limited to one or any combination of those enumerated in W.I.P.O. Publication No. 2018/089939 A1 (see, e.g., page 9, line 27 through page 10, line 5), as well as U.S. Patent Publication No. 2019/0269823 A1 (see, e.g., paragraph 27), incorporated herein in their entirety for all purposes. The surface modification may include one or more, two or more, three or more, or even four or more surface modifications. The surface modification may be modulated using ions, temperature, incubation time, or any combination thereof. Other examples of surface modifications may include, without limitation, coating with a sizing agent, ion exchange, coating with primer, coating with an amino functional material, treatment to provide an oxidized surface (e.g., plasma treatment, corona treatment, ozone treatment, and acidic/basic treatment, plasma deposition, chemical vapor deposition, dip coating, melt-blending, spin coating, spray-on coating), treatment to provide hydroxyl groups on the surface (which can react or provide improved adhesion with the polymer matrix), or any combination thereof. Surface modifications may be applied to filaments after formation of the filaments, after assembly of filaments into fibers, after assembly of fibers into fibrous bundles, or any combination thereof.

Surface modifications may be particularly advantageous when using glass fibers or other high modulus fibers. The surface modification may introduce one or more chemical moieties or functional groups to a surface and/or inner layer of filaments. For example, the treatment may provide an oxidation of the surface or hydroxyl groups on the surface of the filaments. The modification may catalyze or otherwise accelerate a polymerization reaction of monomer or prepolymer in contact with the fiber for forming a polymer. For example, a sizing agent or primer may provide both improved adhesion between an polymeric material and the fibers, and act as a secondary catalyst for the polymerization of monomers in a polymeric material. The functional groups added to filaments may include amino functional materials. Surface abrasion of the glass fiber can lead to enhanced solubility at the surface by disrupting a glass network on the surface. Such disruption may be achieved by acid/base etching, mechanical abrasion, or with plasma oxidation/reduction.

The surface modification may include coating with a sizing agent. The sizing agent may function as a coupling agent, compatibilizer, barrier, or any combination thereof. The sizing agent may function to enhance bonding with polymeric material, increase reinforcement dimensions, modulate hydro-diffusion access/rates to the reinforcement elements, or any combination thereof. The sizing agent may be applied to surfaces of filaments, fibers, fibrous bundles, fibrous composites, composite elements, or any combination thereof.

The sizing agent may include metal coating, ceramic coatings, polymeric coatings, inorganic salt coating, metal phosphates coating (e.g., phosphates of Fe, Ca, Mg, Zn, Ni, the like, or any combination thereof), or any combination thereof. Examples of suitable metal and ceramic coatings may include, but are not limited to, those enumerated in U.S. Pat. No. 10,525,169 B2, incorporated herein by reference in its entirety for all purposes (see, e.g., column 25, lines 49-59). The metal coating may absorb into the filaments. The metal coating may react with water to provide basic/alkaline products that can act as buffering and degradation control agents for the polymeric material and/or filaments.

The sizing agent may be less soluble than the filament material or insoluble. Cladding may refer to a process whereby filaments are coated with a less soluble or insoluble layer.

Examples of suitable polymeric coatings may include silanes, amino silanes, lysine, polyamines, amino acids, polyamino acids, or any combination thereof. Suitable methods for applying sizing agents to filaments may include those disclosed in U.S. Pat. No. 10,525,169 B2 (see, e.g., column 25 line 60 to column 26 line 13), and W.I.P.O. Publication No. 2019/217748 A1 (see, e.g., pages 82-83), incorporated herein by reference in their entirety for all purposes.

The surface modification may include one or more compatibilizers (“coupling agent”). The compatibilizer may function to promote compatibility and/or chemical adhesion among a composite article, constituents thereof, and an environment surrounding the implant. Compatibility may improve interfacial bonding, mechanical properties, physical properties, osseointegration, or any combination thereof. The compatibilizer may provide improved adhesion between two components by Van der Waals forces, ionic bonding, covalent bonding, or any combination thereof. The compatibilizer may enhance or provide a chemical bond between two or more components of a composite article (e.g., filaments and polymeric material). The interfacial bond strength between two or more regions or components (e.g., cores, reinforcement elements, bone) may be enhanced through the addition of compatibilizers.

The compatibilizer may be applied to incorporated into one or more containment bags, filaments, fibers, fibrous bundles, fibrous composites, composite elements, polymeric material, cores, outer regions, or any combination thereof. For example, a first compatibilizer may be applied to filaments to enhance interaction with polymeric material and a second compatibilizer may be used on an outer region to enhance interaction with bone. Examples of suitable compatibilizers and methods of applying the same may include, but are not limited to, those enumerated in U.S. Pat. No. 10,525,169 B2 (see, e.g., column 59, lines 30 through 52), incorporated herein by reference in its entirety for all purposes. Compatibilizers may be applied to an exterior surface of a containment bag to improve adhesion to a bone, accelerate osseointegration, or both. It may be particularly advantageous to treat a containment bag with osseoconductive material. For example, a containment bag may have fused-silica with aluminum oxide applied by dip-coating.

The surface treatment may include one or more ion exchanges (“stuffing”). The ion exchange may function to achieve a desired degradation rate, ion release profile, glass strengthening, or any combination thereof. The ion exchange may be particularly advantageous when glass filaments are employed. The ion exchange may create a gradient of degradation rates and/or packing density of ions in the filament. The ion exchange may replace ions in the filament with ions that are a different size than the ions already present in the filament. For example, a first ion exchange step may introduce a larger ion into filaments in order to create a packing density that allows a second layer that has a smaller ion to increase packaging density by filling in more spaces or displacing the initial ions. As another example, the gradient could start with a coating of a smaller ion followed by a larger ion or an equivalent sized ion to give a variable gradient. It may be particularly advantageous, for increased glass strength, to replace ions with larger ions, which puts stress on surrounding atomic bonds. For soluble glasses, ions such as sodium cation or phosphate anion may be replaced with less soluble ions, such as calcium, magnesium, iron cations, silicate anions, or any combination thereof.

The ion exchange may be performed by contacting filaments with molten salts containing ions which will be exchanged with ions already present in the filaments. The molten salt may include pure salts, a eutectic mixture of salts with lower melting points, or both. Examples of suitable eutectic mixtures may include, but are not limited to, those disclosed in U.S. Patent Publication No. 2019/0269823 A1 (see, e.g., paragraphs 43 to 46).

The ion exchange process may be performed by dipping and/or soaking the glass fibers in molten salts of desired metals. The time of soaking may be used to control the amount of ion exchange. The soaking time could range from 1 second, 1 minute, 1 hour, 24 hours, 1 week, or even multiple weeks, depending upon the desired properties of the glass fibers. Multiple soakings and/or multiple soaking times with different ions in sequence may be utilized. The ion exchange may include one or more exchanges. There may be one or more, two or more, three or more, four or more, or even five or more ion exchanges.

The ion exchange may replace at least a first ion in the glass with at least a second ion in the glass. The second ion may be larger than, smaller than, or the same size of the first ion. Any number of subsequent ion exchanges may be performed. Any subsequent ions may replace any other ions in the glass. Any subsequent ions may be larger than, smaller than, or the same size of any other ions in the glass.

The surface modifications may result in variable solubility throughout a cross-section of a filament. Dual-solubility filaments may be obtained by various processes including but not limited to ion exchange, surface abrasion, cladding, or any combination thereof. It has been shown that phosphate-based glass fibers (PGF), for example, may be used to control the properties of biodegradable composites for potential application as bone fracture fixation devices and can be doped with Si or Fe (Acta Biomaterialia, 8:4, April 2012, pp. 1616-26). Dual-solubility filaments may have a diameter of 1 μm or more, 5 μm or more, 10 μm or more, 50 μm or more, or even 100 μm or more. Dual-solubility filaments may have a diameter of 1 mm or less, 800 μm or less, 600 μm or less, 400 μm or less, or even 200 μm or less. The filaments may have one or more layers of variable solubility. The one or more layers may surround a core. Core of a filament, when used in the context of surface modifications, may refer to the centrally located columnar portion of a filament, viewable in a cross-section of the filament. The variation of the solubility may be gradual, with no defined core or layer boundaries. The core of the filament may have a thickness of about 5%, or more, 10% or more, 20% or more, 25% or more, or even 50% or more, of the diameter of the filament. The core of the filament may have a thickness of about 95%, or less, 80% or less, 75% or less, 65% or less, or even 55% or less, of the diameter of the filament.

An example of preparing dual-solubility glass may proceed as follows. Phosphate glass filaments (solubility of 6.2*10-4 mg/cm2-h; 4% Na, 38% Ca, 48% P, 5% B, and 5% Fe) may be immersed in a eutectic mixture of Ca(NO3)2, Mg(NO3)2, and NaNO3 in a furnace kept at roughly 250° C. for 6 hours. The overall solubility of the glass fibers, as derived by ion analysis in solution, may reduce to about 7.3*10-5 mg/cm2-h. The concentration of sodium, as derived by X-ray photon spectroscopy, may decrease to about 1.5%. The concentration of calcium, as derived by X-ray photon spectroscopy, may increase to about 49.1%. The concentration of magnesium, as derived by X-ray photon spectroscopy, may increase to about 1.4%. As another example, phosphate glass filaments of the composition were immersed in a eutectic mixture of Mg(NO3)2 and KNO3 in a furnace kept roughly at 350° C. for 24 hours. The overall solubility of the glass fibers, as derived by ion analysis in solution, may reduce to about 5.3*10-5 mg/cm2-h. The concentration of sodium, as derived by X-ray photon spectroscopy, may decrease to about 1.2%. The concentration of magnesium, as derived by X-ray photon spectroscopy, may increase to about 1.2%. Upon X-ray photon spectroscopy (XPS), it was found that the concentration of sodium decreased to whereas the concentration of potassium increased to 1.9%, and concentration of magnesium increased to 0.9%.

The core of the glass fiber can have a solubility ranging from 1*10⁻⁹ mg/cm²-h to 1*10⁻¹ mg/cm²-h. By way of example but not limitation, the solubility of silicate could include the solubilities for anhydrous sodium metasilicate and the pentahydrate, which are 210 g/l at 20° C. and 610 g/l at 30° C., respectively or other types of silicate. Examples are as follows: Amorphous silicate glasses are only slightly attacked by water at ambient temperatures and can be solubilized only at elevated temperature and pressure (ca. 150° C. and >5 bar). The solutions are infinitely dilutable with water. Silicate powders obtained by water evaporation from silicate solutions are readily soluble in water. Amorphous silica which precipitates when alkaline solutions are neutralized has a water solubility of 115 mg/l at 25° C. and neutral pH (Morey et al. 1964). Depending on both pH and concentration the respective solutions contain varying proportions of monomeric tetrahedral ions, oligomeric linear or cyclic silicate ions (e.g., di- or trisilicate ions) and polysilicate ions of three-dimensional structure. See, for example, http://www.inchem.org/documents/sids/sids/solublesilicates.pdf. The density could range from less than 1.24 g/cm3 to 750 kg/m3 or higher. The layers of the filament can have a solubility that is 10% or more, 30% or more, 50% or more, 70% or more, or even 100% or more, lower than the original filament. The layers of the filament can have a solubility that is 1,000% or less, 700% or less, 500% or less, or even 300% or less, lower than the original filament. The layers of the filament can have a solubility that is 10% or more, 30% or more, 50% or more, 70% or more, or even 100% or more higher than the original filament. The layers of the filament can have a solubility that is 1,000% or less, 700% or less, 500% or less, or even 300% or less, higher than the original filament.

The surface modifications may change the refractive index of the filament. The refractive index may be tailored to create a modification of the light transfer in the filament. Tailoring the refractive index may function to allow various types of therapies and energy distribution to be conducted through the composite article. One example of the change in the index of refraction could be done with ion doping or ion swapping. Various ions could be used including strontium. The tailoring of refractive index may be applied to any layer and/or core of the filament. This could allow the core, outer surface, any number of middle layers, or any combination thereof to have the same or different properties. The properties may include chemical durability, mechanical properties, functionalizing (e.g. ion doping), or any combination thereof. This could change the gradient over the distance of the glass (or coating) given the layer thickness. Tailoring the refractive index may provide for tailored degradation. The degradation rate may be step-wise throughout the layers and/or core, proceed along a gradient, or both. The refractive index can vary depending on the structure and surface modifications of the filament. The refractive index may vary depending on temperature. For example, the filament may exhibit a refractive index outside a body of a patient and a different refractive index when located within a body of a patient. The refractive index may be about 1.2 or more 1.4 or more, 1.6 or more, or even 1.8 or more. The surface modifications may change the index of refraction by 0% or more, 5% or more, 10% or more, 15% or more, or even 20% or more. The surface modifications may change the index of refraction by 40% or less, 30% or less, 35% or less, or even 25% or less.

The refractive index may be modulated such that the light is totally internally reflected. By way of example but not limitation, light may be shined on the surface such that a propagated wave strikes the medium boundary at an angle larger than a particular critical angle with respect to the normal to the surface. This may be utilized with any type of glass shape (e.g., a rod, a sheet, a sphere, etc.) so that light treatment or other targeted treatments could be applied to the surfaces. Total internal reflection of light could propagate with minimal energy loss when the it moves from a medium having a given refractive index to a medium having a lower refractive index. The light may be internal reflected off of the sides of the fiber, so the light strikes the sides of the fiber at angles greater than the critical angle. Optical fibers usually include a core with a cladding material to lower the index of refraction. In this case the modifications to the surface can work as the cladding. For example, pure silica (n=1.444) can be wrapped around a core of dropped silicon (n=1.4475). The modification of the surface can change create a layer with an index of refraction that can range from 1.2-2.65. The layers allow for like transfer and the coatings can change the index of a refraction from greater than 1.33 (to much higher) so that light can travel through these layers and could reflect or effect controlled angle and wavelengths.

The surface modifications to the glass may also change the roughness of the filaments such that reflection and refraction can be modulated when energy is propagated through the filaments. The filaments can act as a way to restrict one or more dimensions of the energy (sound, light, EM waves, etc.) given the construction.

Ions and surface modifications can also be configured to release biological elements for activity for treatment or radiopaque material in order to identify the device using imaging techniques. Using this technique, the surface can also be roughened in order to help with changing the mechanical properties and/or biocompatibility and/or integration into the bone.

In a composite that is in contact with water or a fluid, the filaments may lose contact with the polymer material due to glass dissolution. This phenomenon may lead to rapid degradation of mechanical properties, even though the weight loss is minor. An agent (“bulking agent”) that can swell or expand as it absorbs water can be useful in maintaining contact between the fibers and the polymeric material. An example of a suitable bulking agent may include alginate. Alginates are linear polysaccharides composed of D-mannuronopyranosyl and L-guluronopyranosyl units. Sodium alginate is formed by treatment of alginic acid (derived from natural sources) with sodium hydroxide or sodium carbonate. A coating can be made consisting of various layers of sodium alginate and a suitable calcium salt which is insoluble at neutral pH, but soluble at low pH. As the glass fibers dissolve, they may release phosphoric acid which can dissolve the calcium salt. The calcium ions generated can diffuse in the alginate coating, forming a swellable alginate gel, with improved mechanical properties compared to sodium alginate. Thus, the multi-layered coatings can act as bulking agent, as a buffering agent, and as a dynamic method of maintain interfacial contact as fibers degrade.

The composite article, containment bags, filaments, fibers, fibrous bundles, fibrous composites, composite elements, polymeric materials, cores, outer regions, sizing agents, compatibilizers, or any combination thereof may include pores. The pores may promote bone ingrowth into the composite article. The size of the pores may determine the rate of bone ingrowth. The size of the pores may increase over time as the composite article and/or components thereof bioabsorb. The average pore size may be about 0.0005 μm or more, about 0.0010 μm or more, about 0.010 μm or more, or about 0.10 μm or more. The average pore size may be about 1000 μm or less, about 500 μm or less, about 100 μm or less, about 50 μm or less, about 10 μm or less, about 5 μm or less, or about 1.0 μm or less.

The composite article, region(s), cores, outer region, fibers, fibrous bundles, fibrous composites, polymeric material, or any combination thereof may have a surface roughness with a pore size between 0-10000 um, or 10 um-500 or 20-300 um or 100-700 um or 10 nm-50 nm, or 15-30 nm. In one preferred form of the invention, the composite article, composite article, region(s), cores, outer region, fibers, fibrous bundles, fibrous composites, polymeric material, or any combination thereof may have a porosity between 0-90% of the surface, more preferably 0-50%. As will hereinafter be discussed, the one or more reinforcement elements are selected by the physician so as to provide the composite article with the desired size, stiffness and strength. Thus, and as will hereinafter be discussed, the physician may select from a variety of different reinforcement elements, each having a particular composition and length, and preferably deliver those reinforcement elements sequentially to the patient, whereby to provide the composite article with the desired size, stiffness and strength. The physician may, optionally, size the reinforcement elements to the appropriate length.

The composite article, cores, outer region, filaments, fibers, fibrous bundles, fibrous composites, polymeric material, containment bag, or any combination thereof may include a coating comprising quantum dots. The quantum dots may allow for thermal decomposition or radiopaque material.

A plurality of filaments may be grouped together to form fibrous bundles. The fibrous bundles may comprise about 5 or more, 10 or more, 100 or more, or even 1,000 or more filaments. The fibrous bundles may comprise about 1,000,000 or less, 500,000 or less, 100,000 or less, or even 10,000 or less filaments. The filaments may be continuous filaments, chopped filaments, or both. Continuous filaments may extend a length of a fiber. The final composite element (e.g., composite implant), reinforcement elements, or both may have a fiber volume of about 20% or more, 40% or more, or even 50% or more. The final composite element (e.g., composite implant), reinforcement elements, or both may have a fiber volume of about 90% or less, 80% or less, 70% or less, or even 60% or less. Fiber volume may refer to the volume of filaments occupying a final composite element, reinforcement elements, or both.

The fibrous bundles may be twisted. The twist may be clockwise (S), counterclockwise (Z), not twisted (0), or any combination thereof. The twist rate may be about 0 or more, 3 or more, 5 or more, 7 or more, or even 9 or more twists per inch. The twist rate may be about 20 or less, 18 or less, 16 or less, 14, or less, or even 12 or less twists per inch.

The fibrous bundles may assemble to form a textile. The textile may be woven, non-woven, or both. The non-woven textile may be fabricated from randomly oriented filaments, chopped filaments, or both. The woven textile may be biaxially woven, triaxially woven, quadaxial woven, or any combination thereof.

The filaments may assemble together to form a fiber. The fiber may comprise a plurality of filaments arranged together. The filaments may be continuous throughout the fiber. The filaments may be arranged in axial alignment with one another. The filaments may be nested. The fiber may be kept together by a twist, sheath, encapsulation, or any combination thereof. The filaments may be coated or not coated before being formed into a fiber. Different fibers may have the same or different fiber volumes. Each of the fibers may include about 10 or more, 20 or more, 50 or more, 100 or more, 1,000 or more, 2,000 or more, 4,000 or more, 10,000 or more, 20,000 or more, or even 25,000 or more filaments. Each of the fibers may include about 55,000 or less, 45,000 or less, 35,000 or less, or even 30,000 or less filaments.

The fibers may be constructed of a variety of different filaments with different properties. For example, a thermoplastic filament may be interwoven within portions of a higher modulus filament in order to facilitate handling during cutting operations with a hot knife or, through the use of a heat gun, to reduce filament damage during storage.

The composite article may be fabricated from a plurality of reinforcement elements. The plurality of reinforcement elements may be fabricated from one or more filaments, fibers, polymeric material, or both. The reinforcement elements may include fibrous bundles, fibrous composites, composite elements, polymeric material, or any combination thereof. The reinforcement elements may function to sequentially build-up to form the composite article.

The fibers may be arranged together to form fibrous bundles. The fibrous bundles may be separated and/or coated by a polymeric material. Fibrous bundles may be combined in any one or combination of the following ways to produce one or more other reinforcement elements. One or more continuous axial fibrous bundles may be run through the center of a braiding table and other continuous fibrous bundles (bias bundles) may be braided over the axial fiber bundle at an angle to the longitudinal axis of the axial fibrous bundles. The angle may be ±0° or more 5° or more 15° or more 25° or more, 35° or more or even 45° or more. The angle may be ±90° or less 75° or less, 65° or less, or even 55° or less. 75°. One or more continuous axial fibrous bundles may be run through a bed of a braiding table and other continuous fibrous bundles (bias filament bundles) may be braided to interlock the axial fibrous bundles at an angle. The angle may be ±0° or more 5° or more 15° or more 25° or more, 35° or more or even 45° or more. The angle may be ±90° or less 75° or less, 65° or less, or even 55° or less. 75°. The fibrous bundles may or may not be impregnated with polymeric material. The fibrous bundles may have a fiber volume of about 10% to about 90%, about 40% to about 70%, or even about 50% to about 60%. The remaining volume may be occupied by polymeric material (“polymeric material”).

The reinforcement elements may be coated, impregnated, suspended, or any combination thereof with a polymeric material. Polymeric material may direct the mechanical properties of the composite article. The polymeric material may be low modulus, high ductility to achieve flexibility. Some properties of the filaments may direct the wettability of the polymeric material in the process of creating reinforcement elements, including fiber volume, fiber orientation, weave/braid/twist, or any combination thereof. Some properties of the reinforcement elements may direct the stiffness of the reinforcement elements, including fiber volume, fiber orientation, weave/braid/twist, or any combination thereof. The reinforcement elements may have a tensile and/or compressive strength that is greater than a polymeric material in which it is located. The reinforcing elements may include a mix of different materials with varying tensile strength, compressive strength, degradation profiles, or any combination thereof. The geometry and/or material makeup of the reinforcement elements may be variable along a length of the reinforcement element so that physical properties may be variable along the length.

The construction of the reinforcement elements may direct the mechanical properties of the composite article. The construction of the reinforcement elements may include fiber count, fiber volume, fiber orientation, weave/braid/twist, cross-sectional shape, cannulation, thickness, or any combination thereof. The reinforcement elements may have a cross-sectional thickness of about 10 μm to 10 mm. The reinforcement elements may have an aspect ratio of from about 1:1 to about 1:10,000.

The reinforcement elements may have a cell size. The cell size may be determined by a characteristic periodic repeat pattern in a fabric weave. Altering cell thickness and cell height within a region may modulate the properties of the composite.

The reinforcement elements may have a cross-sectional shape including round, ovoid, elliptical, triangular, quadrangular, rhomboid, pentangular, hexangular, octangular, cruciform, lobed, the like, or any combination thereof. The reinforcement elements may include a cannulation. The reinforcement elements may have a cross-section aspect ratio that is defined by the cross-sectional length of the reinforcement element along its major axis and the cross-sectional length of the reinforcement element along its minor axis. The cross-section aspect ratio may be the ratio of the minor access to major access. The cross-section aspect ratio of the reinforcement element may be 1:1, greater than 1:1, greater than 1:2, greater than 1:3, greater than 1:5, between 1:1 and 1:30, or even between 1:1 and 1:100.

A plurality of particulates may be included in the reinforcement elements. The particulates may include granules, segments, nanotubes, whiskers, nanorods, the like, or any combination thereof.

The combination of different reinforcement elements in different orientations within the composite article may result in a tougher composite. The individual reinforcement elements may act as a unit to resist mechanical loads. The reinforcement elements may redirect fracture lines, dissipate energy, absorb energy, or any combination thereof, resulting in a ductile failure mode. Interstitial space between reinforcement elements, which includes polymeric material, may redirect fracture lines, dissipate energy, absorb energy, or any combination thereof, resulting in a ductile failure mode.

Two or more reinforcement elements may be braided together. The braiding may include diaxial, triaxial, quadaxial, or any combination thereof.

Fibers and polymeric material may be combined in any one or combination of the following ways to produce reinforcement elements. One or more polymeric materials may be formed into polymeric filaments, co-mingled with the fibers, and drawn through a heated die to cause melting of the polymeric filaments and flow of the matrix filaments around the fibers, thus embedding the fibers in a polymeric material. The fibers may be pultruded by drawing them through a bath of one or more polymeric materials through a heated die to accelerate curing of the polymeric material. The shape of the die, in either method, may determine the cross-sectional shape of the reinforcement element.

An axial reinforcement element may be wrapped with a polymeric material coated reinforcement element at an angle to the longitudinal axis of the axial reinforcement element to create a reinforcement element that comprises both axial and bias reinforcement elements. The angle may be ±0° or more 5° or more 15° or more 25° or more, 35° or more or even 45° or more. The angle may be ±90° or less 75° or less, 65° or less, or even 55° or less.

One or more reinforcement elements may be combined with one or more other reinforcement elements to create composite articles. One or more reinforcement elements may be worked by nesting, binding, interlacing, interlocking, or any combination thereof.

As referred to herein, nesting may mean axially aligned and stacked. Nesting of axial reinforcement elements may provide the advantage of the physical properties of the axial bundle in tension and compression but provides for additional polymeric material between discrete reinforcement elements, which allows for slippage between elements, reducing brittle failure.

As referred to herein, binding may mean axially aligned and wrapped with one or more other reinforcement elements. Binding of axial reinforcement elements may constrain buckling of the axial reinforcement elements and increase column strength and stiffness. Bias reinforcement elements in the binding layer may provide for transmission of transverse loads through the composite and may be helpful in applications subjected to torque off-axis to the axial load.

As referred to herein, interlacing (“interlocking”) may mean axially aligned and interlaced with one or more other reinforcement elements. The interlacing may include braiding, weaving, knitting, stitching, pinning, or any combination thereof one or more reinforcement elements with one or more other reinforcement elements. Interlacing between reinforcement elements may provide for slippage between reinforcement elements and may interrupt fracture planes in the polymeric material between reinforcement elements. This reduces crack propagation by absorbing and redirecting energy in the composite and provides toughness, thus reducing brittle failure. Interlacing between axial elements in the composite may help transmit loads between reinforcement elements, provides bias reinforcement elements for absorbing transverse loads, and may interrupt fracture planes in the composite material by placing reinforcement elements in areas that would normally be comprised of only polymeric material. Interlacing between layers of reinforcement elements may transfer loads between layers and also interrupts fracture planes in the composite material by placing reinforcement elements in areas that would normally be comprised of only polymeric material. When placed under torsional loads, the interlacing between layers may transmit torque through the thickness of the composite material more efficiently than a stacked laminate, because the reinforcements can transfer loads from the inner to outer regions of the composite material and vice versa.

Working the plurality of reinforcement elements by nesting, binding, interlacing, interlocking, or any combination thereof may contribute to the toughness of the composite. Working of the plurality of reinforcement elements may make a new larger reinforcement elements. The one or more reinforcement elements may define one or more cores and/or one or more outer regions of a composite article. The properties of the composite article may be determined by combining one or more reinforcement elements of the same or different architectures and/or materials in various hierarchal constructions. The reinforcement elements may include one or more fillers, porogens, therapeutic agents, visualization agents, osteoconductive agents, toughening agents, degradation agents, or any combination thereof.

The reinforcing elements may be in the format of one or more rods, sheets, particulates, or any combination thereof.

The rods and/or sheets may be flexible. The rods may be substantially elongate members. The rods may be fabricated of a plurality of fibers held together by an outer sheath of a textile or film, a plurality of fibers compacted together, a plurality of fibers held together by a polymeric material, or any combination thereof.

The sheets may comprise filaments formed into a textile, incorporated into a film, or both. The textile may be woven, braided, knit, non-woven, the like, or any combination thereof. The reinforcement properties of the textile may be modified by changing the material, orientation (angle), length, shape, volume, twist, or any combination thereof of the fibers within the textile. The film may be formed by suspending series of reinforcement elements in polymeric material and curing, at least partially, the polymeric material. The sheets may be substantially planar members. The sheet may act as a sheath, wrapping around other elements of a composite article.

The sheets may be formed into tape. The tape may have a rectangular and/or elliptical cross-section. The tape may be wound around one or more reinforcement elements. The tape may be wound around axial reinforcement elements. The tape may be wound at an angle of ±0° or more 5° or more 15° or more 25° or more, 35° or more or even 45° or more. The tape may be wound at an angle of ±90° or less 75° or less, 65° or less, or even 55° or less. The angle may be acute.

The sheets may be worked into derivative formats including tubes, rolls, or both. The tubes and/or rolls may be flexible. The tubes may be fabricated by joining two opposing ends of a sheet. The rolls may be fabricated by rolling a sheet. The roll may be radially compressed, radially expanded, or both.

The one or more reinforcing elements may be introduced into a containment bag by means of a delivery catheter or sheath. The reinforcement elements may have sufficient column strength to allow longitudinal delivery into a containment bag by pushing. The reinforcement elements may be flexible. The reinforcement elements may be pre-cured prior to introduction into a patient. The reinforcement elements may have smooth outer surfaces and/or tapered ends to facilitate movement past one another and/or intervening structures while being delivered into the containment bag. The sheets, in the form of rolls, may be radially compressed by other reinforcement elements and/or intervening structures while being delivered into the containment bag. The sheets may comprise resilient elements (e.g., resilient rings) to assist their subsequent radial expansion when entered into a containment bag. The resilient elements may be thermosensitive or have a shape memory.

The tubes may be placed concentrically one within another. The rolls may be placed concentrically within the tubes. The reinforcement elements may include surface projections for improved integration with the polymeric material. The surface projections may be formed by interspersing chopped and/or non-continuous filaments in random orientations throughout the length of the reinforcement elements such that at least a portion of the randomly oriented filaments extend beyond the outer surface of the reinforcement elements.

The plurality of reinforcement elements, when employed in a composite article, may include a plurality of axial reinforcement elements, a plurality of bias reinforcement elements, or both. The axial reinforcement element may extend along a longitudinal axis of a composite article. The bias reinforcement elements may extend at an angle to the longitudinal axis of the axial reinforcement elements, the longitudinal axis of the composite article, or both. The angle may be ±0° or more 5° or more 15° or more 25° or more, 35° or more or even 45° or more. The angle may be ±90° or less 75° or less, 65° or less, or even 55° or less. The angle may be acute.

The bias reinforcement elements may function as tensors splitting into additional axial support and torsional and burst resistivity. The bias reinforcement elements may function to add hydrostatic pressure to draw polymeric material into a center of non-planar reinforcing elements and to aid in the final cross-sectional shape without altering the overall cross-sectional footprint. It may be particularly advantageous to provide the bias reinforcement in the form of a tape.

The axial reinforcement elements may provide column stiffness, compression strength, compression stiffness, or any combination thereof. The bias reinforcement elements may provide flexural stiffness and torsional strength. The bias reinforcement elements may transmit torque from the inside of the composite to the outside of the composite. Interlocking with bias reinforcements may allow transmission of forces through the thickness of the composite without the typical delamination between layers seen with laminate composites. Bias reinforcement elements may translate torque from a proximal portion of a composite article to a distal portion of a composite article. For example, a driver engaged with a cannulation and causing a composite article to turn may generate torque that is translated by bias reinforcements.

The composite article may have a ratio of axial fibers to bias fiber of about 10:90 or more, 10:75 or more, 10:55 or more, 10:35 or more, 10:15 or more, or even 10:10 or more. The composite article may have a ratio of axial fiber to bias fiber of about 90:10 or less, 75:10 or less, 55:10 or less, 35:10 or less, or even 15:10 or less. The ratio of axial fibers to bias fibers may depend on the desired properties.

The plurality of reinforcement elements may be selected by physician to provide the composite article with the desired size, mechanical properties (e.g. stiffness and strength), or both. Thus, the physician may select from a variety of different reinforcing elements, each having a particular composition, length, or both and deliver those reinforcing elements sequentially into a bone of the patient, whereby to provide the composite article with the desired size and attributes of stiffness and strength.

The composite article may comprise about 5% or more, 10% or more, 20% or more, 40% or more or even 60% or more reinforcement elements, by volume. The composite article may comprise about 95% or less, 85% or less, 75% or less, or even 65% or less reinforcement elements, by volume. The plurality of reinforcement elements may be modified by changing the materials, dimensions, shape, and surface characteristics of the reinforcement elements.

The reinforcing properties of the reinforcement elements may be modified by changing the orientation, volume, twist, angle, volume, layup, or any combination thereof of the fibers within the reinforcing elements. The fibers may be set at an acute angle to intersecting fibers in order to strengthen the reinforcing elements. The angle may be ±0° or more 5° or more 15° or more 25° or more, 35° or more or even 45° or more. The angle may be ±90° or less 75° or less, 65° or less, or even 55° or less.

The reinforcing properties, and degradation profiles, of the one or more reinforcing elements may be modified by changing the material, dimensions, shape, orientation, volume, and surface features of the fibers, filaments, and/or particulates used to form the one or more reinforcing elements. Where the reinforcement elements comprise a textile, its reinforcing properties and degradation profile may be modified by changing the materials, orientation, length, shape, volume, twist, and angle of the fibers and filaments within the textile of the reinforcing elements. The fibers and/or filaments in a textile of a reinforcing element may be set at an acute angle to intersecting fibers and filaments. The angle may be ±0° or more 5° or more 15° or more 25° or more, 35° or more or even 45° or more. The angle may be ±90° or less 75° or less, 65° or less, or even 55° or less.

The composite may comprise one or more polymeric materials (“matrix material”, “resin”, “injectable matrix”, “interface”, “coating”). The polymeric material may function to provide a medium to bind and hold one or more reinforcement elements into a solid configuration. The polymeric material may be a low modulus material. The polymeric material may serve to transfer load; offer protection to the reinforcement elements from environmental damage; provides finish, texture, durability, functionality, or any combination thereof to the composite material; or any combination thereof. The polymeric material may comprise polymer resins that are thermoplastic, thermoset, or both. The polymer may be synthetic, organic, or both. The polymer may be flowable. The polymer may be injectable. The polymeric material may be biodegradable, non-degradable, or both. The polymeric material may biodegrade and/or bioabsorb in response to regional (bodily or environmental) stimuli. The stimuli may include water, saline, naturally or artificially introduced enzymes, or any combination thereof. The polymeric material may comprise cross-linked chemistries, non-cross-linked chemistries, or both. The polymeric material may be flowable. The polymeric material may contain a biocompatible solvent. The solvent may reduce viscosity to allow the polymeric material to flow easier and aid in encapsulating filaments and/or reinforcing elements. The solvent may rapidly diffuse so as to facilitate or provide stiffening or curing of the composite structure. The solvent may act as a porogen. The solvent may also be used to alter the porosity of the polymeric material. The polymeric material may be polymerizable in situ. The polymeric material may include a multi-component polymer system that is mixed immediately prior to introduction into the patient. Exemplary and non-limiting polymers may include polyurethanes, acrylics, polyesters, polyamides, polyamines, polyaramides, polyaryletherketones, polysulfones, polyolefins, biopolymers, or any combination thereof. The polymeric material may include a bioactive filler, a degradation agent, a deposition agent, or any combination thereof. Optionally, the polymeric material may include other typical ingredients used in composites, and other formulated products such as paints, inks, adhesives and sealants. These other ingredients may be pigment or filler particles, surfactants, defoamers, and other commonly known and used additives.

The polymeric material may be molded, extruded, and/or shaped using other techniques in order to achieve the desired shape. By way of example but not limitation, the shape of polymeric material may be a pin, a sphere, a rod, and/or a prism with three or more sides.

Composite strength is determined, in part, by how well the reinforcements in the composite are wet out by the matrix. Poor wetting of the fibers can result in gaps between the giver and the final solidified matrix. These gaps become flaws in the final composite which will reduce its ultimate strength. In general, as a composite gets larger, it becomes more difficult to penetrate the plurality of fibers with matrix. By building the structure in a hierarchal fashion from discrete elements, each smaller element may or may not be pre-wet with matrix prior to assembly. The modularity of the construction also allows for matrix to flow freely between reinforcement elements providing for improved wetting.

By way of example but not limitation, biodegradable polymer polymeric materials may include those enumerated in U.S. Patent Publication No. 2019/0175734 A1 (see, e.g., paragraph 239), as well as those enumerated in W.I.P.O. Publication No. 2015/095745 A1 (see, e.g., page 71, line 8 through page 72, line 6), incorporated herein by reference in their entirety for all purposes. By way of further example but not limitation, suitable non-biodegradable or non-bioabsorbable materials may include polyolefins, polyamides, polyesters, polyimides, polyetheretherketone (PEEK), polyetherketoneketone (PEKK), carbon fiber, metal, ceramics, glass, or any combination thereof. By way of further example but not limitation, the polymeric material may include those enumerated in U.S. Patent Publication No. 2019/0269823 A1, (see, e.g., paragraph 242), incorporated herein by reference in its entirety for all purposes.

The polymeric material may comprise a thermoplastic and bioabsorbable glass (bioglass). The bioabsorbable glass may assist the ambient hydrolytic breakdown of biodegradable polymers which are currently only broken down at elevated temperatures. The use of bioabsorbable glass may be particularly advantageous where effective hydrolysis must take place at bodily temperatures or temperatures acceptable to the local environment. Polymers specifically sensitive to this inventive approach include, but not limited to, are polylactic acid homo and copolymers, polycaprolactone, polybutylene succinate homo and copolymers, polybutylene succinate co adipate, polyethylene glycol terephthalate co adipate, poly butylene glycol adipate co terephthalate, etc. Typically, catalytic levels or sub 10% levels of bioabsorbable glass is used in these applications in powder, chopped fiber or continuous fiber form. The thermoplastic composite system may also include 0.1% to 20% of an inorganic material that, when immersed in an aqueous environment at temperatures of about 5° C. to about 40° C., and having a pH from 5.5 to 8, will result in the aqueous micro-environment of the composite component to change by at least 0.5 pH, and preferably about 1.5 to 3 pH, thus accelerating the hydrolytic breakdown of the matrix (e.g., polyester or polyester polyurethane) components so that the molecular weight is reduced by a factor of 4 to 20 in the defined timescale, making the residual low molecular weight fragments extremely brittle in nature and susceptible to microbial attack.

Hydrolytic breakdown of the composite can be catalyzed both by acid and basic conditions. Acid conditions can be generated from phosphate anion release in the soluble glasses which is moderated by the choice of cations with sodium/potassium being more soluble that calcium/magnesium and with aluminum/iron the least soluble Alkaline conditions are generated from the sodium ion release in the bioglass silicate glass compositions. Ion release has to be facilitated through the presence of an aqueous media.

Hence glass materials which generate either soluble acidic or basic ions in an aqueous environment under ambient conditions can catalyze the hydrolytic breakdown of PLA polymer.

The matrix may include a non-reactive polyester plasticizer in the amount of about 0-30% of the weight of the matrix, or 30% and above. The plasticizer may comprise non-reactive aliphatic polyesters. If desired, a thickening agent may be added to control the viscosity and can be achieved through a hot mixer of resin and polymers.

If desired, the surface of the polymeric material may be modified using various techniques. By way of example but not limitation, the surface of the polymeric material may be modified using ion exchange, a therapeutic agent may be added to the surface of the polymeric material (e.g., so that the polymeric material can be used to facilitate drug delivery), the polymeric material may be modified to comprise a radiopaque material (e.g., for imaging), the polymeric material may be modified to have varying index of refractions in order to present a gradient for tailored degradation, etc. The coatings and materials may also tailor the mechanical and chemical properties within the ranges discussed above.

In one form of this invention, the polymer polymeric materials, both crosslinkable thermosets and thermoplastics, can be derived from green or bio-based sources, such as soy or corn. Bio-based polymers are materials which are produced from renewable resources. Bio-based polymers can be biodegradable (e.g., polylactic acid) or nondegradable (e.g., biopolyhethylene). Similarly, while many bio-based polymers are biodegradable (e.g., starch and polyhydroxyalkanoates), not all biodegradable polymers are bio-based (e.g., polycaprolactone). Bio-based polymers offer important contributions by reducing the dependence on fossil fuels and through the related positive environmental impacts such as reduced carbon dioxide emissions. Bio-based can be directly derived from agricultural feedstocks such as corn, soy, potatoes, and other carbohydrate feedstocks, or by bacterial fermentation processes by synthesizing the building blocks (monomers) from renewable resources, including lignocellulosic biomass (starch and cellulose), fatty acids, and organic waste. In another strategy bio-based material, such as starch, is mixed with non-bio-based materials, such as polyethylene. Example of synthetic bio-based polymers include polylactic acid, polyglycolic acid, polycaprolactone, P4HB, P3HB, polybutylene succinate, bio-polyethylene, and mixtures and copolymer thereof. Examples of natural bio-based natural polymers include starch, chitosan, chitin, collagen, spider silk, pullulan, cellulose, gelatin, and alginates. Natural and synthetic bio-based polymers can be mixed to provide materials with novel properties. Biobased polyurethane can be derived by reacting bio-based polyol (for example from corn, vegetable oil, or castor oil) and/or bio-based isocyanate (from example from soy protein).

The polymeric material may have a molecular weight ranging from about 10 or more, 250 or more 500 or more, or even 1,000 or more. The polymeric material may have a molecular weight ranging from about 100,000 or less, 10,000 or less, or even 5,000 or less. The polymeric material may have a viscosity of about 1 cps or more, 10 cps or more, 50 cps or more, 100 cps or more, 250 cps or more, or even 500 cps or more. The polymeric material may have a viscosity of about 100,000 cps or less, 50,000 cps or less, 25,000 cps or less, or even 10,000 cps or less. It may be particularly advantageous for the polymeric material to have viscosities less than 3,000 cps. By way of example but not limitation, the matrix can have a polydispersion index (pdi) of less than about 1.1, 1.5, 2, or even 2.5.

The flowable matrix can be a crosslinkable thermoset polymer such as a polyurethane, epoxy, polyurea, polyurea urethane, acrylate, acrylate urethane, propylene glycol fumarate, polycarbonate, polystyrene, or polycitrate esters. They may contain degradable bonds such as polyesters, including polylactic acid, polyglycolic acid, polyhydroxybutyric acid, polycaprolactone, polymalic acid, polydioxanes; polyanhydrides such as polysebacic acid or polyadipic acid; polyamides such as polyiminocarbonates and polyaminoacids; phosphorus based degradable bonds such as polyphosphates, polyphosphonates, and polyphosphazenes; or other biodegradable polymers such as polycyanoacrylates, polyorthoesters, polyacetals, or polydihydropyrans.

In one aspect, the polymeric material may comprise polyurethane. The polyurethane may be produced by reaction of isocyanates having at least two reactive functional groups per molecule (difunctional or polyfunctional) with a molecule having two or more active hydrogen groups (difunctional or polyfunctional) capable of reacting with the isocyanate.

Examples of suitable isocyanates may include but are not limited to those enumerated in U.S. Pat. No. 10,525,168 B2 (see, e.g., column 25, lines 7 through 29), as well as U.S. Patent Publication No. 2019/0175734 A1 (see, e.g., paragraph 157), incorporated herein by reference in its entirety for all purposes. When a biodegradable implant is desired, aliphatic isocyanates are generally favored. The isocyanate index of the isocyanate can range from 5% to 60%, preferably from 15% to 45%. When a non-biodegradable implant is desired, aromatic isocyanates are generally favored.

The molecule having the hydrogen groups may include primary and secondary aliphatic hydroxyls and amines; primary, secondary, and aromatic amines; aliphatic and aromatic thiols; urethane and urea groups; polyols; or any combination thereof. Examples of suitable molecules having the hydrogen groups may include but are not limited to those enumerated in U.S. Pat. No. 10,525,168 B2 (see, e.g., column 25, lines 40 through 51), incorporated herein by reference in its entirety for all purposes. Examples of suitable polyols may include but are not limited to those enumerated in U.S. Pat. No. 10,525,168 B2 (see, e.g., column 26, lines 33 through 58), incorporated herein by reference in its entirety for all purposes.

The polyol may have active hydrogen functionality ranging from at least 2 to about 6, preferably from 2 to about 4. For example, ethyleneglycol and 1,4-butanediol/THF have a functionality of 2; glycerol, trimethylpropane, and propylene glycol have a functionality of 3; pentaerythritol has a functionality of 4; sorbitol and mannitol have a functionality of 6; and sucrose has a functionality of 8. The hydroxyl number of the polyol can range from 40 to 1000, preferably from 100 to 800. The polyol may have a molecular weight (in kilodaltons) ranging from about 50 to about 50,000, or even from about 100 to about 3,000.

The polyols may include at least one bioabsorbable group to alter the degradation profile of the resulting branched, functionalized compound. The bioabsorbable groups may include, but are not limited to, groups derived from glycolide, glycolic acid, lactide, lactic acid, caprolactone, dioxanone, trimethylene carbonate, and combinations thereof. The bioabsorbable groups may be present in an amount ranging from about 7% to about 95%, or even about 50% to about 90% of the combined weight of the multifunctional compound and bioabsorbable groups.

The isocyanate may be reacted with the polyol to produce a prepolymer. The resulting prepolymer may then be stored until combined with additional polyol to form the final polyurethane product. Reaction of the urethane prepolymer with polyol to form the final polyurethane product generally requires a catalyst to provide convenient working and cure times. The catalyst may include amine compounds organometallic complexes, or both. Examples of suitable amine catalysts may include, but are not limited to, those enumerated in U.S. Pat. No. 10,525,168 B2 (see, e.g., column 27, lines 30 through 48), incorporated herein by reference in its entirety for all purposes. Examples of suitable organometallic catalysts may include, but are not limited to, those enumerated in U.S. Pat. No. 10,525,168 B2 (see, e.g., column 27, lines 49 through 60), incorporated herein by reference in its entirety for all purposes.

The composite may include one or more fillers. The filler may be included in a matrix or reinforcement element. The filler may function to contribute functionality to the composite that would not necessarily be provided by the matrix, reinforcement element, or both, alone. The filler may provide porosity, bone ingrowth surfaces, enhanced permeability, enhanced pore connectivity, or resistivity to water permeation, or any combination thereof. The porosity and/or compressive properties of the polymeric material may be modified by using the filler. The composite article may become porous after implantation so as to aid the resorption and bone healing process. The pores may have a diameter from about 50 μm to about 500 μm. It may be particularly advantageous to provide for pores having a diameter of about 100 μm to allow bone ingrowth. The pores may be interconnected. This porosity can be generated by various mechanisms including the preferential resorption of filler. For example, a bone may absorb materials such as calcium sulfate, α-tricalcium phosphate, bioglass or, the like. The filler may be in the form of fibers, flakes, particulate, extractable liquids, or any combination thereof. The filler may be present in a polymeric material, reinforcement elements, or both in an amount of about 1% or more, 5% or more, 10% or more, 20% of more, 25% or more, or even 30% or more, by volume. The filler may be present in a polymeric material, reinforcement elements, or both, in an amount of about 90% or less, 85% or less, 80% or less, 75% or less, 70% or less, or even 65% or less, by volume. The filler may have mean diameters ranging from about 1 μm to about 20 μm and/or mean lengths ranging from about 1 μm to about 500 μm. The filler may include two or more fillers having different sizing distributions. The filler may be biocompatible, insoluble, osteoconductive, or any combination thereof. The filler may be organic, inorganic, or both. The filler may be in the form of particles, fibers, or both. Examples of suitable fillers may include, but are not limited to, those enumerated in W.I.P.O. Publication No. 2015/095745 A1 (see, e.g., page 52, line 26 through page 53, line 7), as well as U.S. Patent Publication No. 2019/0175734 A1 (see, e.g., paragraph 185), incorporated herein by reference in its entirety for all purposes. The filler may comprise a degradable polymer such as polylactic acid, polyglycolic acid, polycaprolactone, hydroxybutyrate, hydroxypropionic acid, hydroxyhexanoate, co-polymers thereof, or any combination thereof. The degradable polymer may contain one or more inorganic fillers. The inorganic filler may have diameters ranging from about 1 μm to about 20 μm and/or lengths ranging from about 1 μm to about 500 μm. The inorganic filler may have different shapes, including spherical, platelet-shaped, isotropic or anisotropic, fibers (e.g., nanofibers, rods, nanotubes, and nanorods). In addition to or alternative to filler, the composite article may include a biocompatible solvent (e.g., DMSO). The biocompatible solvent may be leached out of the composite article post implantation.

The filler may include one or more porogens. The porogen may include sugars, polysaccharides (e.g., dextran), soluble salts, or any combination thereof. can be incorporated into the polymeric material or reinforcement. The porogen may include crystalline materials in the form of soluble salts. The porogen may dissolve in the presence of water and leave behind pores in polymeric material. The pores may provide for tissue ingrowth, faster matrix degradation, or both, by increasing the surface area of polymeric material exposed to the environment. The porogen may be in the form of a quickly dissolving fiber. The quickly dissolving fiber may create one or more channels for fluid transfer along an intramedullary canal of a bone, increase surface increase surface area for more rapid bio-dissolution of the remaining implant, free up other fillers for timed migration to the local environment, or any combination thereof. The porogen may be present in a polymeric material, reinforcement elements, or both in an amount of about 15% or more, 20% or more, or even 25% or more, by weight. The porogen may be present in a polymeric material, reinforcement elements, or both in an amount of about 50% or less, 45% or less, 40% or less, or even 35% or less, by weight.

The filler may act include one or more therapeutic agents. The therapeutic agent may function to promote bone formation, relieve pain, reduce inflammation, inhibit infection, or any combination thereof. The therapeutic agent may be incorporated into a polymeric material, reinforcement elements, or both. The therapeutic agent may be delivered locally via a carrier vehicle to provide a protective environment, provide target delivery to cells or within cells, provide locally delivery, timed delivery, staged delivery, or any combination thereof. Suitable examples of therapeutic agents may include, but are not limited to, those enumerated in W.I.P.O. Publication No. 2015/095745 A1 (see, e.g., on page 54, line 10 through page 55, line 12), as well as U.S. Patent Publication No. 2019/0175734 A1 (see, e.g., paragraph 192), incorporated herein by reference in its entirety for all purposes. The therapeutic agent may include bone growth activating factors, such as those enumerated in W.I.P.O. Publication No. 2015/095745 A1, (see, e.g., on page 55, lines 17-22), as well as U.S. Patent Publication No. 2019/0175734 A1 (see, e.g., paragraph 194), incorporated herein by reference in its entirety for all purposes. The therapeutic agent may include inorganic material processed by the body as a vitamin such as Fe, Ca, P, Zn, B, Mg, K, Mn, Ce, Sr, the like, or any combination thereof. The therapeutic agent may be tuned for consistent release. The therapeutic agent may be tuned by incorporating into a solubilizing component having a predictable solubility.

The filler may include one or more visualization agents. The visualization agent may function to enhance visibility while imaging the composite article. The visualization agent may be in the form of particles or liquid. By way of example but not limitation, where the physician may be using fluoroscopy to view the bone being treated and the composite article, the polymeric material may include the visualization agents enumerated in W.I.P.O. Publication No. 2015/095745 A1 (see, e.g., on page 55, line 27 through page 56, line 10), incorporated herein by reference in its entirety for all purposes.

The filler may include one or more osteoconductive agents. The osteoconductive agent may function to provide porosity, bone ingrowth surfaces and enhanced permeability or pore connectivity or resistivity to water permeation. The osteoconductive agent may be biocompatible, insoluble, osteoconductive particles, or any combination thereof. The osteoconductive agent may be in the form of particles, short fibers, or both. Examples of suitable osteoconductive agents may include, but are not limited to, tricalcium phosphate, orthophosphates, monocalcium phosphates, dicalcium phosphates, tricalcium phosphates, tetracalcium phosphates, amorphous calcium phosphates, biodegradable/bioabsorbable glasses, or any combination thereof.

The filler may include one or more toughening agents. The toughening agent may function to toughen a materials resistance to fracture by absorbing energy. The toughening agent may be in the form of chopped fiber, flake, particulate, or any combination thereof. The toughening agent may be of a lower modulus material than the polymeric material. Higher modulus materials may be included to disperse stresses throughout the matrix by interrupting crack propagation within the structure. Liquids may also be added to the matrix to act as plasticizers which may lower the modulus of the matrix and act to toughen the composite.

The filler may act to control degradation rate of the composite article. Degradation (i.e., reduction of molecular weight, mass, strength, or any combination thereof) may proceed until the material disintegrates into constituent particles or into material with a low enough molecular weight such that enzymes can digest the remnants. Different materials may degrade at different rates under different environmental conditions such as temperature, moisture level, and/or pH. The degradation of the composite (and/or its components) can also be slowed (or accelerated) by the addition of high aspect ratio platelet-shaped additives, water-reactive compounds, and/or inorganic or organic buffering agents to the matrix. Suitable inorganic bases can be added, such as salts and oxides of alkaline metals, including basic mono-, di-, and tri-phosphates, calcium oxide, calcium hydroxide, magnesium oxide, magnesium hydroxide, bioglass flakes, calcium phosphate, beta tricalcium phosphate, hydroxyapatite, potassium stearate and sodium stearate. Particles of metals such as magnesium, iron, titanium, and zinc or metal alloys, such as magnesium base alloys, can also be added. Other possible fillers include water-reactive particles, such as calcium oxide or cobalt chloride. Organic bases, such as polyamines, bispidines, and proton sponges, are examples of self-buffering agents. The self-buffering or degradation controlling agents can be encapsulated in a micro- or nano-capsule and are released under certain physiological conditions.

However, heretofore, a method has not existed for to control the onset and rate of degradation under non-ideal material conditions. As an example, current biodegradable materials are not stiff enough for most load-bearing applications contemplated herein. The addition of a soluble glass, such as soluble glasses that are made primarily with phosphate, will stiffen the material, increasing the number of applications for which the material is suited. However, phosphate glasses are hygroscopic and, therefore, will begin to lose mass from the outside in, losing intimal contact with the surrounding matrix and thus diminishing the benefits of the composite article. The application of the layering techniques disclosed herein allow for tailoring and controlling the internal environment in relationship to the external environment so as to increase the shelf life and working life of the composite in order to make the product practical.

The composite material may include one or more washout channels. The washout channels may function to facilitate degradation. The washout channels may do so by exposing components of the composite material to aqueous environments that promote degradation. The washout channels may allow degraded materials to flow out of the composite material and into a surrounding environment. The washout channels may be formed into a composite material during its construction and/or may be formed by degradation of one or more components of a composite material. Washout channels formed by degradation of components of a composite material may extend axially, transversely, at an angle with respect to axial, or any combination thereof. The washout channels may include interstitial passages between filaments. The interstitial passages may arise from degradation of polymeric material. The rate of washout channel propagation may be controlled by selection of polymeric material, selection of filament material, interstitial space between filaments, coatings and/or barriers around composite articles, reinforcement elements, filaments, or any combination thereof. The washout channels may prevent buildup of acidic conditions by allowing acidic solutions to disperse and/or flow out of the composite article. The washout channels may prevent buildup of ions by allowing ionic solutions to disperse and/or flow out of the composite article. The washout channels may provide for bone and/or tissue ingress and/or growth. The washout channels may provide for bone and/or tissue growth within the first 6 weeks, 4 weeks, or even 2 weeks after the composite article is introduced into a patient.

A further adaption of the technology is to have the reinforcement component made up of several components which have differing chemical compositions where at least one of the components generates ions in an aqueous environment to move the pH from about 7 by at least 2 pH units in either an acidic or basic manner. For example, carbon fiber can be combined with either a bio absorbable silicate or phosphate fiber or ground powder to increase the mechanical strength properties but does not hydrolytically degrade whilst the silicate or phosphate fiber or ground powder provides the hydrolytic breakdown catalyst function.

In one preferred form of the invention, the composite article, region(s), outer core, outer region, core region, reinforcement elements, and/or polymeric materials are capable of degrading into sub-components engineered such that the host area (e.g., the body or the local environment) is beneficially affected. The duration, intensity, and sequence of the release of remnants of the degradation process can be designed to produce pH shifts in a local environment or to release other compounds into the local environment. For example, during the degradation of a material structure, there may be a rapid release of remnants for a burst of either acidic or basic pH shift, followed at a later period of time by the release of buffering solutions to re-alter that environment.

A polysaccharide such as, but not limited to, chitosan, chitosan/PLA, or Chitin can also be used as a coating on containment bag, composite article, region(s), outer core, outer region, core region, reinforcement elements, and/or matrix. Wang et al developed a method for thermally-induced phase separation to prepare polyglycolic acid PGA-(chitosan hybrid matrices with low toxicity). In one instance, this method may be used in both sizing and matrix composition. The weight ratio PGA to chitosan can range between 1:9 (PGA to Chitosan) to 9:1, 7:3, or 3:7. This technique may also be performed using PLA (using the same rations as previously mentioned). The pore size ranges from 0.001 Angstroms to 500 Angstroms or more. The pore size can help to determine the rate of degradation. The sizing and/or polymeric material may also comprise additional therapeutic molecules, or molecules for facilitating wound healing (or otherwise to deliver localized treatments).

### C. Method

The present disclosure provides for a method of treating a bone fracture. The composite implant of the present disclosure may be disposed within an intramedullary canal of a bone or any other suitable opening in a bone. The composite implant may function as a splint. The composite implant may carry stress generated during patient activity. In this manner, the composite implant may enable a bone fracture to heal, provide fortification of bone, provide augmentation of bone, or any combination thereof. The composite implant may achieve these aforementioned effects with minimal inconvenience to a patient. The components of the composite implant may be introduced sequentially into the patient, assembled in-situ, or both thereby allowing the composite implant to be installed using a minimally invasive approach.

First, an access hole may be formed in a bone. The access hole may provide access to an intramedullary canal of the bone. An access port may optionally be disposed in the access hole to facilitate delivering a composite implant through the access hole. The access hole may be formed on a distal side of the bone that is proximal to a fracture. The access hole may be formed at an angle of 0° to 90° to the bone. The access hole may be formed at an acute angle (e.g., about 45°) to the bone. The acute angle may be particularly advantageous in allowing the composite implant to be more easily introduced into the bone. The modular nature of the composite implant allows for the access hole to be smaller than the composite implant. For example, the access hole may be 3 mm in diameter and the composite implant 5 may be 10 mm in diameter. As a result, the composite implant may be employed using a minimally invasive procedure that may be carried out in an office setting or surgicenter setting rather than in a conventional operating room.

Second, a catheter may be introduced through the access hole via the access port. A rotatable flexible rod may be introduced into the bone via the catheter. The rotatable flexible rod and additionally/alternatively liquid and/or gas may be utilized to clean the intramedullary canal by disrupting bone marrow to allow removal of bone marrow (e.g., via suction through the catheter). As a result, a space for the composite implant may be created. The catheter may have markers on its exterior surface so as to allow the physician to determine the position of the containment bag within the bone by direct visualization of the markers on the exterior surface of the catheter. A containment bag may have markers thereon so as to allow the physician to determine the position of the containment bag within the bone by indirect visualization (e.g., fluoroscopy, CT, etc.).

Third, one or more flow restrictor plugs may be introduced into the intramedullary canal, via the access hole, proximal to where the composite implant will be placed. The flow restrictor plugs may be connected to one another. One or more flow restrictor plugs may be utilized. The flow restrictor plugs may be optionally placed in the bone prior to removing or harvesting the bone marrow. Alternatively and/or additionally to flow restrictor plugs, a containment bag may be introduced into the intramedullary canal, the containment bag being releasably attached to the catheter. The containment bag may be flexible and compressible to allow its introduction into the intramedullary canal via a minimally invasive approach. The containment bag may comprise an auxiliary channel to allow monitoring and control of subsequent pressurization with the polymeric material. This auxiliary channel may be parallel to the delivery catheter, or inside the delivery catheter, or the auxiliary channel may be at the distal end of the containment bag. Alternatively, there may be a valve at a distal end of the containment bag, or at other strategic regions of the containment bag, that can limit pressure within the containment bag. The containment bag may be omitted. In this case, the one or more reinforcing elements and polymeric material may be deployed directly into the intramedullary canal (or other opening) in the bone that is being treated, without an intervening containment bag.

Fourth, the bone may be returned to proper alignment.

Fifth, one or more reinforcement elements may be introduced into the intramedullary canal through the catheter via the access hole, so as to build up a reinforcing mass in-situ.

Sixth, one or more reinforcing elements may be sequentially introduced through the access hole, via the catheter, and into the containment bag so as to build up a reinforcing mass in-situ. The flexible nature of the one or more reinforcing elements may allow their delivery into the access hole via a minimally invasive approach. The one or more reinforcing elements may be introduced into the containment bag with the facilitation of a guidewire may be provided to facilitate introduction of the one or more reinforcing elements into the containment bag. The guidewire may be attached to a distal end of the containment bag using an adhesive or other non-permanent attachment means. After the one or more reinforcement elements have been placed in the containment bag, the guidewire may be detached from the containment bag by pulling or twisting the guidewire. Alternatively, the guidewire may be absorbable, in which case it may be left in the patient at the conclusion of the procedure. The guidewire may be used to reduce a fracture prior to delivery of the composite implant. To achieve this, the guidewire may have an enlargement formed at one end, with enlargement being disposed exterior to the bone being treated, and with the opposite ends of guidewire emerging from port. As a result of this configuration, by applying tension to end of guidewire, the fracture can be reduced, and the tensioned guidewire may help support the bone. A fixture may be positioned within the intramedullary canal of the bone, adjacent to enlargement, so as to direct guidewire along the longitudinal channel of the bone and thereby facilitate fracture reduction and delivery of the composite. If desired, the composite implant may be formed out of reinforcement sheets without any reinforcement rods; with reinforcement rods and without any reinforcement sheets; and with a laminated construction comprising both reinforcement sheets and reinforcement rods.

Continuing with step six, the one or more reinforcing elements may include one or more reinforcing sheets and one or more reinforcement rods. A plurality of reinforcing sheets (in the form of tubes, “reinforcing tubes”, may be sequentially inserted into the containment bag, nested concentrically one inside another. Additionally, a plurality of reinforcement rods may be sequentially inserted within the innermost reinforcing sheet. The reinforcement sheets may be delivered to the interior of the containment bag by pushing them out of a delivery tube or, alternatively, by carrying them into the containment bag while held within a delivery tube and then retracting the delivery tube, whereby to expose the reinforcement sheets and allow them to expand. The size and number of flexible concentric reinforcing tubes and reinforcing rods may be selected so as to meet the individual needs of a particular patient. The number of flexible concentric reinforcing tubes utilized in the composite implant, their lengths, their cross-sectional dimensions, the number of reinforcing rods used, their lengths, cross-sectional dimensions, or any combination thereof may be selected according to the individual needs of a particular patient. The number of flexible concentric reinforcing tubes utilized in the composite implant, their lengths, their cross-sectional dimensions, the number of reinforcing rods used, their lengths, cross-sectional dimensions, or any combination thereof, may be selected so as to provide a composite implant having variable stiffness along its length (e.g., a stiffer central region (e.g., 20 GPa) and less stiff distal and proximal ends (e.g., 3 GPa)), whereby to prevent stress risers from being created at the ends of the composite implant. To this end, the reinforcing tubes, and the reinforcing rods, may be preferably provided in a variety of sizes for appropriate selection by the physician; alternatively, the reinforcing tubes and/or reinforcing rods may be sized at the time of use by the physician.

Seventh, a polymeric material may be introduced into the containment bag through the catheter, via the access hole. The polymeric material may be carried by a catheter. The polymeric material may flow over and between the one or more reinforcement structures. The catheter may be removed after introduction of the polymeric material into the containment bag. The polymeric material may be formed from two or more components that are mixed immediately prior to injection into the patient. This may occur through use of a static mixer fed by multiple syringes. The components may be mixed in a bowl and then loaded into a syringe that is connected to the injection tube. The injection tube may be also capable of transmitting an energy wave into the polymeric material in cases where pulsatile flow or the application of vibrational forces is required to aid injecting the polymeric material into the containment bag. Suction may be used to facilitate wetting out of the reinforcement structures by removal of trapped air from the composite. If desired, an expandable device (e.g., a balloon) may be used to provide a radial force to aid in the creation of a single integrated structure. The expandable device (e.g., balloon) may be used to enhance the penetration of the polymeric material into and between one or more reinforcing elements, the containment bag and the bone, and to enhance the interfacial bond between the polymeric material and the one or more reinforcing elements, between the polymeric material and the containment bag, and between the polymeric material and the bone. This solidification may occur through a chemical reaction that proceeds at a rate that allows sufficient time for injection before the viscosity increases to a point where injection is not possible. This time may be five to ten minutes. The solidification may occur within thirty to sixty minutes, although with most chemistries there will be a continuation in strength build-up over a period of hours. In the preferred chemistries the exothermic nature of the reaction is limited to minimize temperature increase in the polymeric material to less than 10° C.

Eighth, the polymeric material may be solidified resulting in a single solidified structure (composite implant) of polymeric material, one or more reinforcing elements, and the containment bag. This solidified structure may be capable of providing support across the fracture while the bone heals. The composite implant may contour to a shape of the intramedullary canal of various types of bones.

Ninth, the patient's wound created to access the bone may be closed up.

The present disclosure provides for a method of fabricating reinforcement elements from filaments, polymeric material, or both. The reinforcement elements may be formed by a molding process. Continuous axial filaments may be positioned inside a mold cavity and a polymeric material may be introduced to flow around and/or impregnate the filaments. Chopped fiber filaments may be introduced into a mold cavity and polymeric material may be introduced to flow around and/or impregnate the filaments.

The present disclosure provides for a method of fabricating a composite implant in the form of a screw. Structural features of the screw may be formed on/in an outer region of a composite implant. The outer region may be applied to a core by coating or over-molding. Over-molding may involve placing a composite core into a mold and over-molding an outer region over the core. The finished geometry of the screw threads may be formed as part of the molding process. A thick outer region may be applied to a core of an implant and then excess material may be removed (machining) to create the threads of the screw. After machining, a thin layer of polymeric material may be applied to the outer region.

Construction of a screw from a composite core. 7.5 mm diameter rods were constructed by embedding 6 bundles of axially aligned continuous glass fibers and binding them with layers of bias continuous glass reinforcement fibers at an angle of ±45 degrees to the axis of the rod. The rod was placed in a lathe and standard machining techniques were used to fabricate threads and a screw head from the rod. Screw threads were machined partway into the bias binding reinforcement layer, leaving some bias layers of the outer region undisturbed by the machining process. This machined rod became a composite core for a bone screw. An outer region consisting of polymeric material filled with a particulate form of a material comprising Calcium Phosphate was applied to the outside of the machined composite core to act as a barrier to prevent ingress of water and control degradation of the core, as well as to provide a compatible surface for interaction with bone. The axial and bias reinforcement elements in the screw run from the screw tip to the screw head. One skilled in the art realizes that other combinations of nested, bound, and interlocked reinforcement elements can be combined to produce cores for screws made in this manner. Another method for making a screw is to over mold or coat a composite core rod with a thick outer region layer. The rod may then be placed into standard machining equipment and the threads and outer geometry may be cut into the outer region alone, leaving the core composite unadulterated.

Reinforcement Elements Made from Extrusion Encapsulated Continuous Glass Fibers. Reinforcement elements were fabricated by melting a thermoplastic polymeric material in a polymer extruder. Continuous glass reinforcing fibers were run through the center of a cross-head die attached to the end of the extruder. When the polymer and the fiber meet inside the cross-head die, the fibers become embedded in the polymer matrix and exit the die in a constant two-dimensional cross-sectional shape determined by the orifice of the die. When the fiber reinforced element exited the die, it was cooled and spooled on a take up reel to be used as a component in other composite cores. Alternatively, the extrudate can be cut to any desired length after cooling.

The mechanical properties discussed herein (e.g., elastic modulus, strain to yield, strain to failure, tensile modulus, the like, or any combination thereof) may be attributed to the structure or the composite material as a bulk structure, fibers, one or any combination of reinforcement elements, or any combination thereof.

### D. Other Fields of Application for the Composite of the Present Disclosure

The composite of the present disclosure may be used for other medical applications. The composite may be used to construct injectable splints, Composite rods, screws, Exam Room Supplies, Wound Care and wound care therapies, Hernia repair, Catheters, Disposable Surgical and Dental Tools, Syringes and Drug Administration, Stents, the like, or any combination thereof. The composites may degrade at the end of their useful life. As a result, medical waste may be managed.

The composite of the present disclosure may be utilized to fabricate surgical and/or dental tools. The tools may include, but are not limited luerlock syringes, fenestrated drapes, needle block foam, needles, paper towels, gauze sponges, prep sponges, drape sheets, scrub brushes, scissors, suture removal tools, suturing tools, surgical clippers, uterine aspirator tubing, adaptors, swabs, Iris scissors, mosquito curved scissors, needle holders, bags, twist ties, Kelly scissors, laxer scissors, forceps, retractors, dissecting scissors, operating scissors, nasal scissors, burr kits, pendontic files, gingirectormy knives, chisels, hatchets, dental scalers, forcep collins, nasal cannula, syringe tips, tubing and hoses are all either single use and thrown away immediately or placed back on the surgical tray to be autoclaved/sterilized.

The composite of the present disclosure may be utilized to fabricate syringes. The composite could be extruded to create a needle, syringe, or both. Degradation may decrease concerns around accidental needle stick injuries and syringe re-use. Additionally, new types of syringes and medical administration techniques are being created that have larger surface areas with the vaccine, medication, drugs such as the nanoneedle array. The composite disclosed here could be used to create that nanoneedle array and contain the drug that will be administered to the patient. There are also capsules that are being designed such as this example: http://www.iflscience.com/health-and-medicine/new-drug-delivery-system-could-replaNFB-injections (visited Nov. 5, 2015), which the composite could be used whereby after the drug is delivered, the drug delivery mechanism could degrade naturally in the body.

The composite of the present disclosure may be utilized to fabricate catheters and/or components thereof in order to provide for degradable at the end of its useful life. The composite could be extruded in a tubular shape that is flexible. The catheter may include any catheter that is used for the continuous administration of intravenous fluids, medications and/or blood products, prolonged parenteral nutrition, chemotherapy, invasive hemodynamic monitoring of arterial blood pressure, central venous pressure and/or pulmonary artery pressure, measurement of cardiac output, hemodialysis (CVC), a peripherally inserted IV catheter (PIVC), a Foley catheter, urinary catheter, pediatric catheter, and peripherally inserted central catheter (PICC), or any combination thereof.

The composite of the present disclosure may be utilized to fabricate single use items typically used in exam rooms. These items may include tongue depressors, surgical blades/scalpels, ointment jars, cotton tipped applicators, plastic cups, nasopharyngeal applicators, drape sheets, tissue wipes (i.e., Kimwipes, Kleenex), gloves, instrument covers (for ears and throat scopes/lights), bed linens, hand towels, towels, facial tissues, sponge bowls, emery boards, table paper, podiatry towels, scopettes, or any combination thereof.

The composite of the present disclosure may be utilized to fabricate wound care products, which aide in protecting and in some cases accelerating the healing processes of an injury to living tissue caused by a cut, blow, or other impact. The wound care products may include pressure bandages, surgical tape, sterile gauze, adhesive bandages, post-surgical bras, elastic skin closures, leukostrips, sutures, non-adherent dressings, flexible bandages, hypo allergenic “paper” tape, non-adhesive membrane pads, non-sterile gauze sponges, porous tape, bandage roll, chest seal, packing strips, sponges, water proof tape, blister pads, debridement pads, wound cleansing sponges, or any combination thereof. Various properties of the materials can be tuned to allow for optimization of the porosity, breathability, adhesive strength, water permeability, absorbance, and flexibility. The wound care product may include additional components that could be added to aide in healing include, but are not limited to, magnesium, zinc, collagen, calcium alginate, biological skin equivalents, topical oxygen, silver products, platelet-rich plasma, platelet-derived growth factors, keratinocytes, and biological components (i.e., extracellular matrix). These additions can be placed on the material through a spray, sputter coating, dip coated, painted, vapor deposition (physical or chemical), chemical techniques, electrochemical techniques, roll to roll coating, thin film deposition, etc.

The composite of the present disclosure may be used for non-medical applications. The composite may be used to construct Fabrics (Military, Parachutes, etc.), Agriculture Mulch Film and Ion Release, Outdoor Sporting, Kitchen Utensils, Bottles, Cafeteria trays, Bags, Diapers, Oil and Gas ion release, Automobile, Packaging (sterile or non-sterile), Abrasives for blasting, parachute fabrication, tents, cabents (cabin with cloth sides), the like, or any combination thereof.

The composite of the present disclosure may be utilized to fabricate abrasive blasting media. The media is propelled as an abrasive material against a surface under high pressure to smooth a rough surface, vice versa, form a material into a shape, remove contaminants from a surface or create a textured pattern on a material. Abrasive blasting may include, but are not limited to, bead blasting, sand blasting, soda blasting, dry ice blasting, shot blasting, non-abrasive blasting methods in ice blasting and dry-ice blasting.

The composite of the present disclosure may be utilized to fabricate utensils (i.e., an implement, container, or other article, especially for household use). The utensils may include, but are not limited to, cutlery, bag, bottle, serving ware, cafeteria tray, the like, or any combination thereof. The utensils would be strong enough for use for tough meats, solid food, but can be tailored to be flexible enough to be used as salad tongs or bendable straws. The material could be used for kitchen utensils, cooking and/or baking, eating utensils, or tools.

The composite of the present disclosure may be utilized to fabricate diapers. Many diapers are made of various layers of materials including a Tissue/non-woven core wrap that creates stability, a Void space that keeps skin dry, an absorbent layer that swells and hold in liquid, wetness indicator to alert when the diaper has wetness, a back sheet that creates a barrier, construction adhesive to hold the diaper together, and a top sheet that helps wick the liquid into the adsorbent layer. One of the main components of the diaper that is not compostable is the absorbent material. The composite of the present disclosure could be used for any component in a diaper, but most importantly, it could be used for the absorbent layer. After its useful life, the diaper can be placed in ambient conditions or conditions that activate the trigger and cause decomposition. This could also be used for sanitary napkins, milk lactation pads, and diapers/disposable underwear required for incontinence.

The composite of the present disclosure may be utilized to fabricate outdoor sporting equipment. The outdoor sporting equipment may include paint ball equipment, fishing and/or crabbing equipment, shooting sports, the like, or any combination thereof. The composite may degrade to reduce environmental impacts of littering thereby reducing injury to wildlife. Paintball equipment may include, but are not limited to, battlefield smoke grenades, ammo (i.e. BBs), CO2 tanks, splatter banners, arm bands, field netting (cable cord or hemmed and grommeted), carabiners, wristbands, signs, netting clips, paint mines, paintball pellets, paint grenades, case shells, gear bags, and practice balls. Fishing equipment may include, but are not limited to, lines, hard baits, soft baits, lures (fresh and salt water), spinner baits, buzz baits, chatter bait, jigs, jig heads, ice fishing lures, fly fishing flies, yarn, dip bait worms, mesh nets, spreader cast nets, bobbers, springs, tip-up line, tip-up accessories, bait pin, fillet knives, the like, or any combination thereof. Crabbing and Clamming equipment may include, but are not limited to, crab harness rings, bait holders, crab trap line, clam net, clam gun, crab trap line weight, the like, or any combination thereof. Bowfishing equipment may include, but are not limited to, bowfishing arrows, bowfishing point, roller arrow rest (typhoon, big head), bow fishing nocks, fin-finder string silencer. Sport shooting and hunting equipment may include but are not limited to clay pigeons, shell casings, shot, wads, clay birds and animals, bolts, arrows, nock, vane, shaft adhesive, archery targets, slugs, sabots, game load, high density pellets (water fowl ammunition), shot shells, jackets, the like, or any combination thereof. The composite may withstand the pressure, recoil, muzzle blast, temperature, and reliability of manufacturing which allows it to be utilized within these fields.

### E. Detailed Description of the Drawings

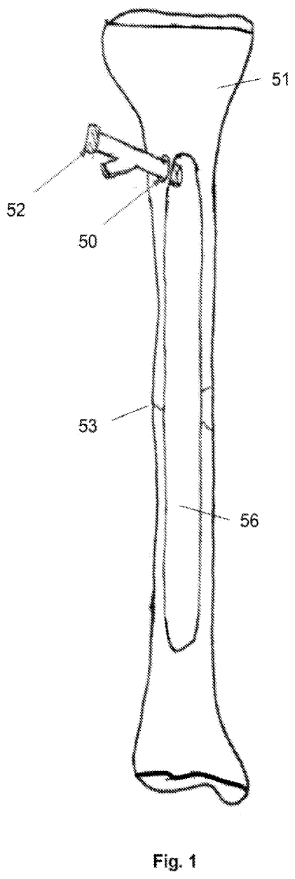

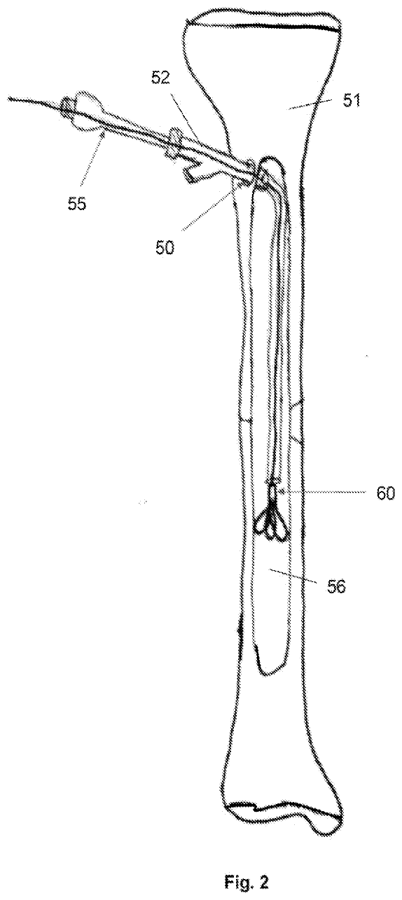

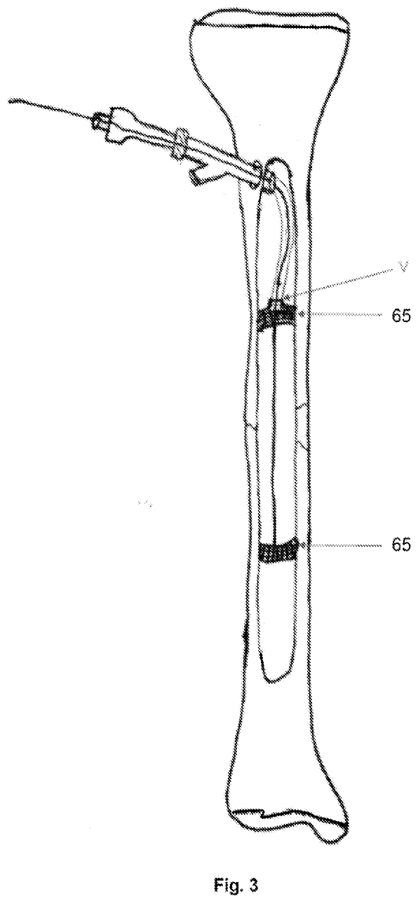

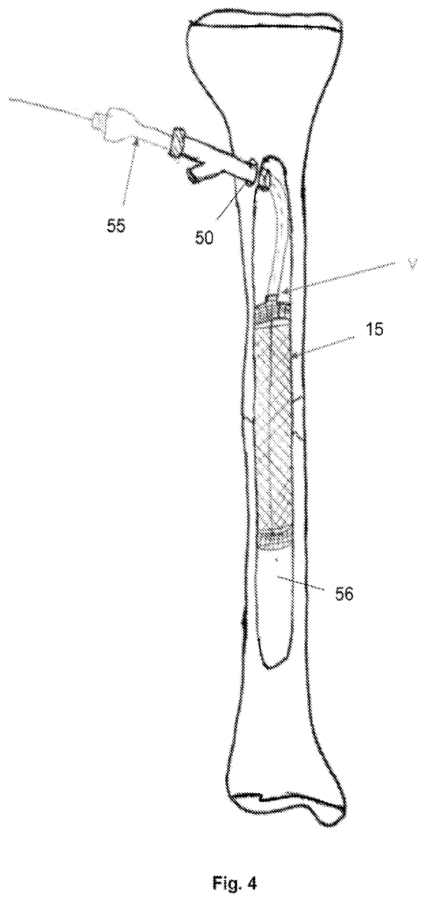

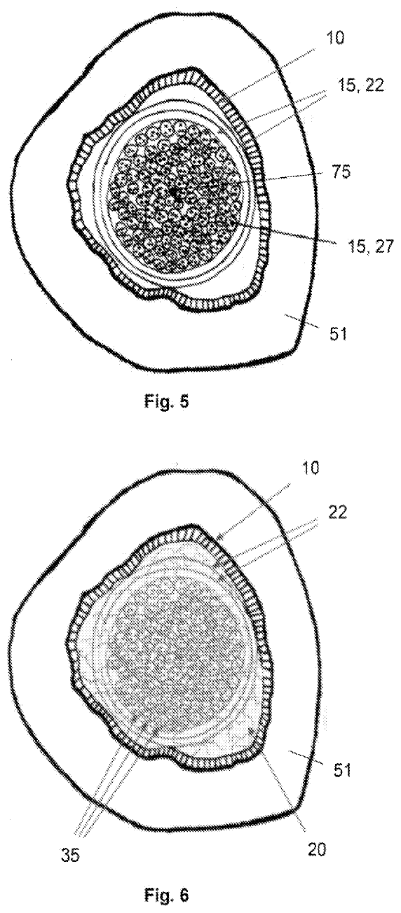

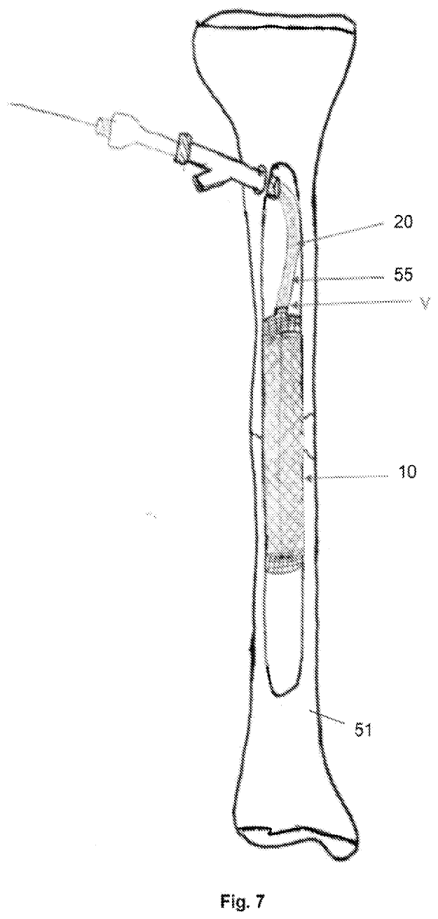

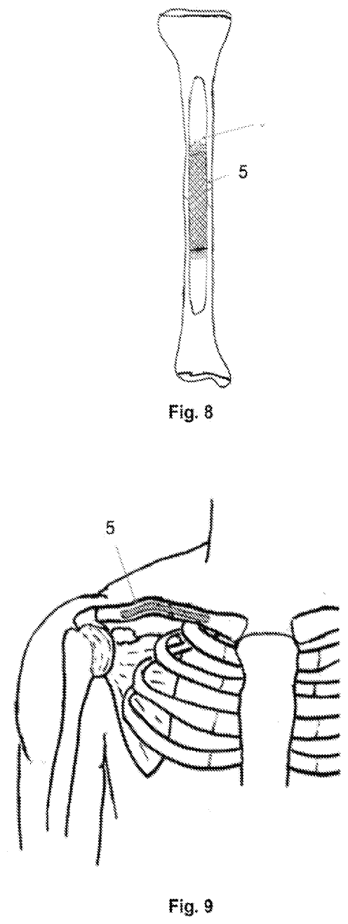

FIGS. 1-9 is a perspective view of a bone and illustrates a method for treating a bone fracture. First, as illustrated in FIG. 1, an access hole 50 is formed in a bone 51. The access hole 50 provides access to an intramedullary canal 56 of the bone 51. An access port 52 is located in the access hole 50 to facilitate delivering individual components of a composite implant 5 through the access hole 50, as shown in FIGS. 5-7. The access hole 50 is formed on a distal side of the bone 51 that is proximal to a fracture 53. Second, as illustrated in FIG. 2, a catheter 55 is introduced through the access hole 50 via the access port 52. A rotatable flexible rod 60, with wire loops at the end, is introduced into the bone 51 via the catheter 55. The rotatable flexible rod 60 and additionally/alternatively liquid and/or gas cleans the intramedullary canal 56 by disrupting bone marrow and/or other biological matter to allow removal thereof (e.g., via suction through the catheter 55). As a result, a space for the composite implant 5 is created. Third, as illustrated in FIG. 3, two flow restrictor plugs 65 are introduced into the intramedullary canal 56, via the access hole 50, proximal to where the composite implant 5 will be placed. Alternatively, a containment bag 10, as illustrated in FIG. 20, may be introduced into the intramedullary canal 56, the containment bag 10 being releasably attached to the catheter 55. The containment bag 10 is flexible and compressible to allow its introduction into the intramedullary canal 56 via a minimally invasive approach. Fourth, the bone 51 is returned to proper alignment. Fifth, as illustrated in FIG. 4, reinforcement elements are introduced into the intramedullary canal 56 through the catheter 55 via the access hole 50. Sixth, as illustrated in FIG. 5, which is a cross-sectional view of a bone, one or more reinforcing elements 15 are sequentially introduced through the access hole 50, via the catheter 55, so as to build up a reinforcing mass in-situ. The reinforcement elements 15 include axial reinforcement elements 27 and bound by sheets 22. The flexible nature of the one or more reinforcing elements 15 allow their delivery into the access hole 50 via a minimally invasive approach. The one or more reinforcing elements 15 are introduced into the containment bag 10 with the facilitation of a guidewire 75 may be provided to facilitate introduction of the one or more reinforcing elements into the containment bag. The guidewire 75 is attached to a distal end of the containment bag 10 using an adhesive or other non-permanent attachment means. After the one or more reinforcement elements 15 have been placed in the containment bag 10, the guidewire 75 can be detached from the containment bag 10 by pulling or twisting the guidewire 75. Alternatively, the guidewire 75 may be absorbable, in which case it may be left in the patient at the conclusion of the procedure. The one or more reinforcing elements 15 include one or more reinforcing sheets 22 and one or more reinforcement rods 35. The sheets 22 are rolled into tubes and are sequentially inserted into the containment bag 10, nested concentrically one inside another. Additionally, a plurality of reinforcement rods 35 are sequentially inserted within the innermost reinforcing sheet 22. Seventh, as illustrated in FIG. 6, which is a cross-sectional view of a bone, and FIG. 7, a polymeric material 20 is introduced into the containment bag 10 through the catheter 55, via the access hole 50. The polymeric material 20 is carried by a catheter 55. The polymeric material 20 flows over and between the one or more reinforcement structures 15. The catheter 55 is removed after introduction of the polymeric material 20 into the containment bag 10. Eighth, the polymeric material 20 is solidified resulting in a single solidified structure (composite implant 5) of polymeric material 20, one or more reinforcing elements 15, and the containment bag 10, as illustrated in FIG. 8. This solidified structure is capable of providing support across the fracture 53 while the bone 51 heals. FIG. 9 illustrates a composite implant 5 introduced into a bone 51 in the same manner as described hereinbefore. As illustrated in FIG. 8 and FIG. 9, the composite implant 5 can contour as necessary to the geometry of the intramedullary canal of a bone 51. The composite implant 5 shown in FIG. 8 is substantially linear, contouring to the shape of a tibia's intermedullary canal. The composite implant 5 shown in FIG. 9 is bent, contouring to the shape of the clavicle's intermedullary canal.

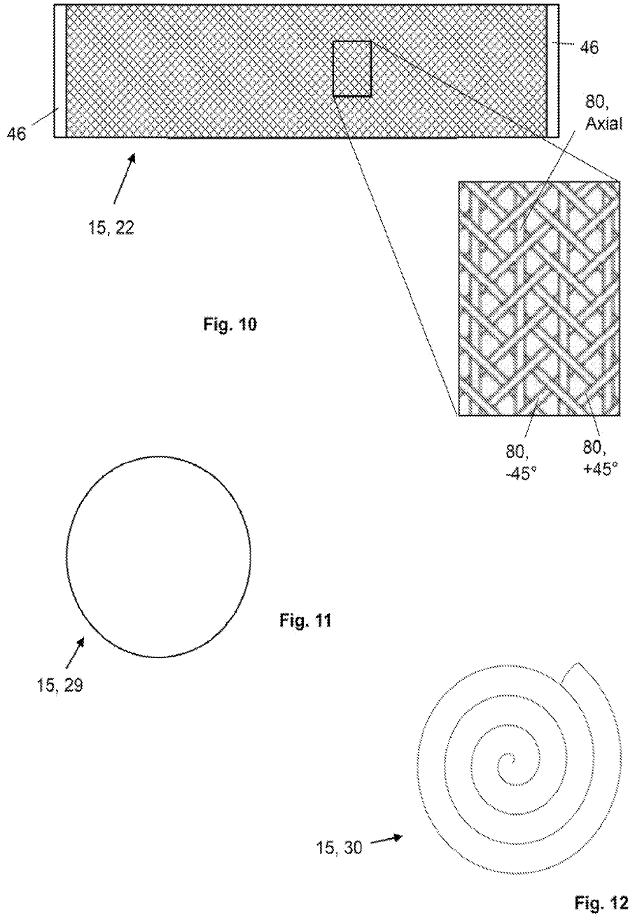

FIG. 10 illustrates a reinforcement element 15. The reinforcement element 15 is in a form of a sheet 22. The sheet 22 comprises fibrous bundles 80 triaxially braided with a series of fibrous bundles 27 arranged axially, a series of fibrous bundles 27 arranged at +45°, and a series of fibrous bundles 27 arranged at −45°. The reinforcement element 15 includes two resilient elements 46 located on opposing ends of the reinforcement element 15.

FIG. 11 illustrates the reinforcement element 15 illustrated in FIG. 10. The reinforcement element 15 is in a form of a sheet 22 formed into a tube 29.

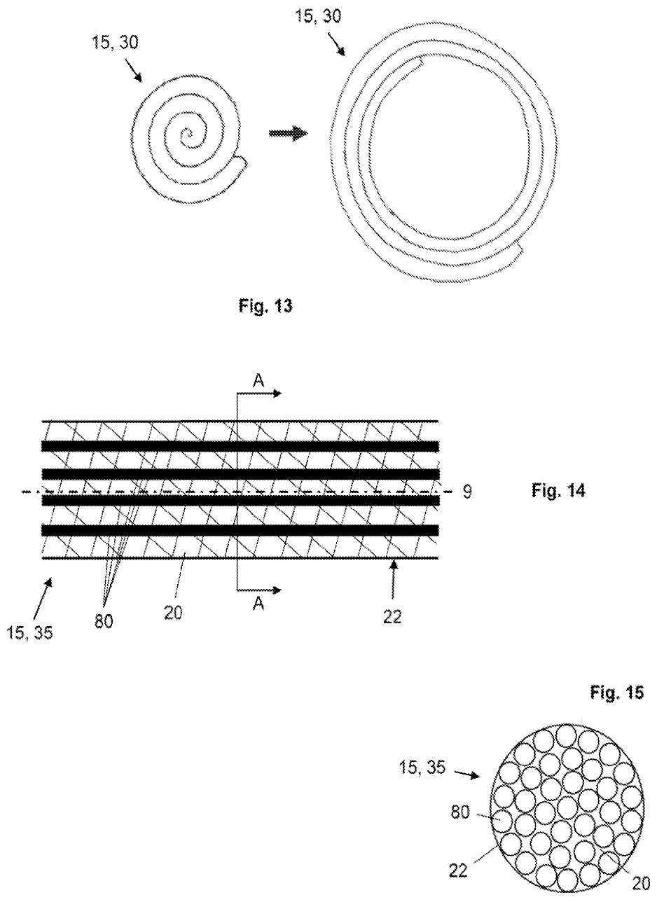

FIG. 12 and FIG. 13 illustrate the reinforcement element 15 illustrated in FIG. 10. The reinforcement element 15 comprises a sheet 22 formed into a roll 30. The roll 30 can be radially compressed (left) or radially expanded (right).

FIG. 14 illustrates a reinforcement element 15. The reinforcement element 15 is in a form of a rod 35. The rod 35 comprises a plurality of fibrous bundles 80 suspended in polymeric material 20 and bound by a sheet 22. The rod 35 includes a longitudinal axis 9, along which the plurality of fibrous bundles 80 are aligned.

FIG. 15 illustrates the reinforcing element 15 illustrated in FIG. 14, along the line A-A. The reinforcement element 15 is in a form of a rod 35. The rod 35 comprises a plurality of fibrous bundles 80 suspended in polymeric material 20 and bound by a sheet 22.

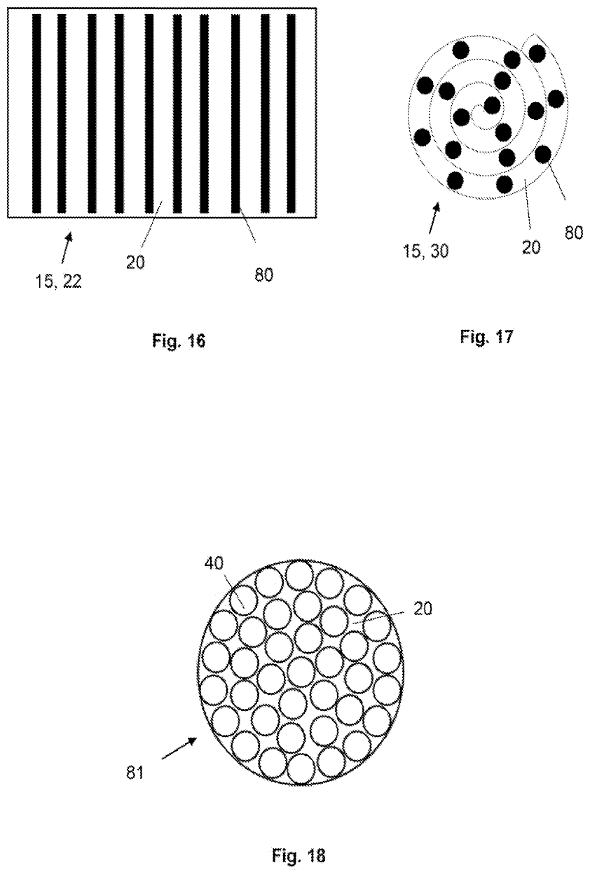

FIG. 16 illustrates a reinforcing element 15. The reinforcing element 15 includes a plurality of fibrous bundles 80 aligned in parallel. The plurality of filaments 40 are suspended in polymeric material 20, forming a sheet 22.

FIG. 17 illustrates the reinforcing element 15 shown in FIG. 16. The reinforcing element 15 includes a plurality of fibrous bundles 80 aligned in parallel. The plurality of filaments 40 are suspended in polymeric material 20, forming a sheet 22. The sheet 22 is formed into a roll 30.

FIG. 18 illustrates a cross-section of a fibrous bundle 80. The fibrous bundle 80 comprises a plurality of filaments 40 suspended and held together in a polymeric material 20.

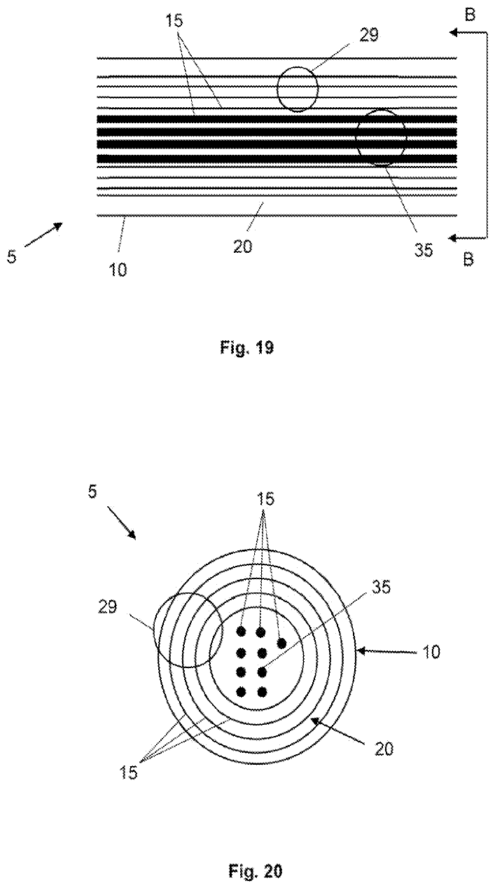

FIG. 19 illustrates a composite implant 5. The composite implant 5 comprises a plurality of reinforcement elements 15. A first set of reinforcement elements 15 are in the form of tubes 29 (see, e.g., FIG. 11). The tubes 29 are arranged concentrically one within another. A second set of reinforcement elements 15 are in the form of rods 35. The rods 35 are nested and located within the innermost tube 29. The reinforcement elements 15 are located within a containment bag 10 and surrounded and/or impregnated with polymeric material 20.

FIG. 20 illustrates the composite implant 5 illustrated in FIG. 19, along line B-B. The composite implant 5 comprises a plurality of reinforcement elements 15. A first set of reinforcement elements 15 are in the form of tubes 29 (see, e.g., FIG. 11). The tubes 29 are concentrically nested one within another. A second set of reinforcement elements 15 are in the form of rods 35. The rods 35 are nested and located within the innermost tube 29. The reinforcement elements 15 are contained within a containment bag 10. Polymeric material 20 is located within the containment bag 10 and surrounded and/or impregnated with polymeric material 20.

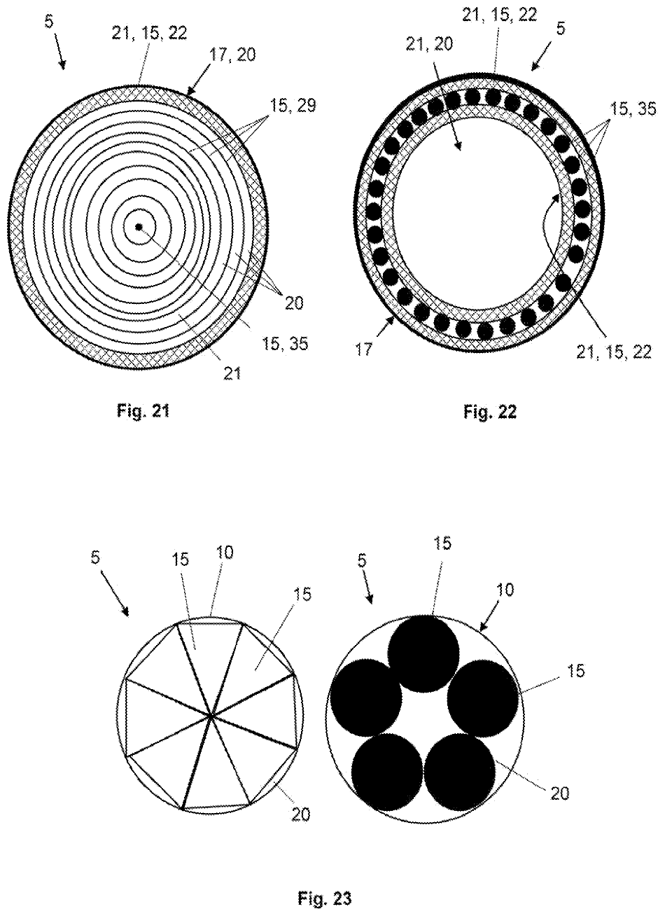

FIG. 21 illustrates a cross-section of a composite implant 5. The composite implant comprises two cores 21 and an outer region 17. One of the cores 21 is formed by concentrically nesting reinforcement elements 15 in the form of tubes 29 around a reinforcement element 15 in the form of a rod 35. The other core 21 is formed by reinforcement elements 15 formed into a sheet 22 (see, e.g., FIG. 10). The reinforcement elements 15 are embedded in layers of polymeric material 20. The outer region 17, comprising polymeric material 20, surrounds the two cores 21.

FIG. 22 illustrates a cross-section of a composite implant 5. The composite implant 5 comprises three cores 21 and an outer region 17. The innermost core 21 is formed by polymeric material 20. The next core 21 is formed of reinforcement elements 15 formed into a sheet 22 (see, e.g., FIG. 10). The next core 21 is formed of axial reinforcement elements 15 in the form of rods 35. The outermost core 21 is formed of reinforcement elements 15 formed into a sheet 22 (see, e.g., FIG. 10). The outer region 17, comprising polymeric material 20, surrounds the two cores 21.

FIG. 23 illustrates cross-sections of two composite implants 5. The two composite implants 5 comprise eight (left) and five (right) reinforcement elements 15, respectively. The reinforcement elements 15 of both composite implants are nested together. The eight reinforcement elements 15 (left) have a triangular cross-section and the five reinforcement elements 15 have a circular cross-section. The reinforcement elements 15 are contained within a containment bag 10 and the containment bag 10 is filled with polymeric material 20. It can be seen that the cross-sectional shape of reinforcement elements 15 direct the number of reinforcement elements 15 that fit within the containment bag 10, the packing density of the containment bag 10, the volume of polymeric material 20 that fills the containment bag 10, or any combination thereof. The composite implant 5 (left) includes a lower volume of polymeric material 20 as compared to the composite implant 5 (right). The higher degree of interfacial contact between reinforcement elements 15 (left) may provide for a composite implant 5 with a higher degree of rigidity. The lower degree of interfacial contact between reinforcement elements 15 (right) and larger volume of polymeric material 20 between reinforcement elements 15 may provide for more slip between reinforcement elements 15.

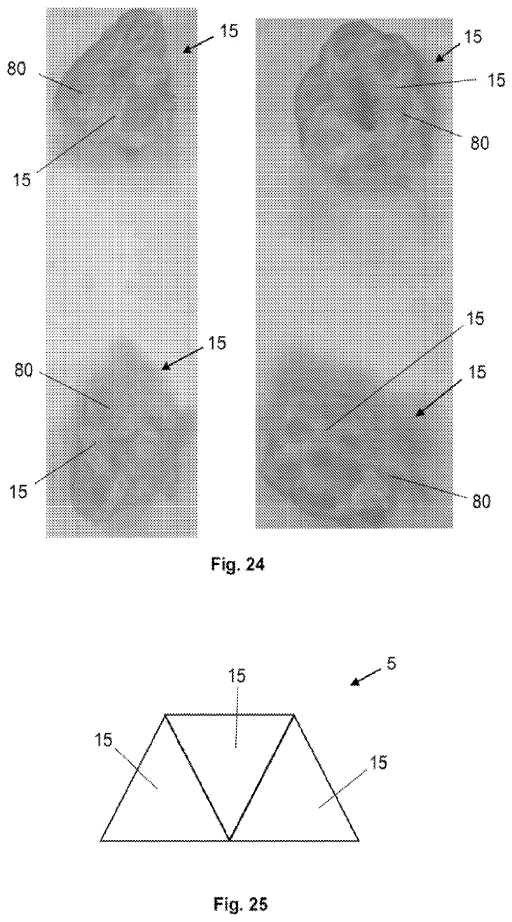

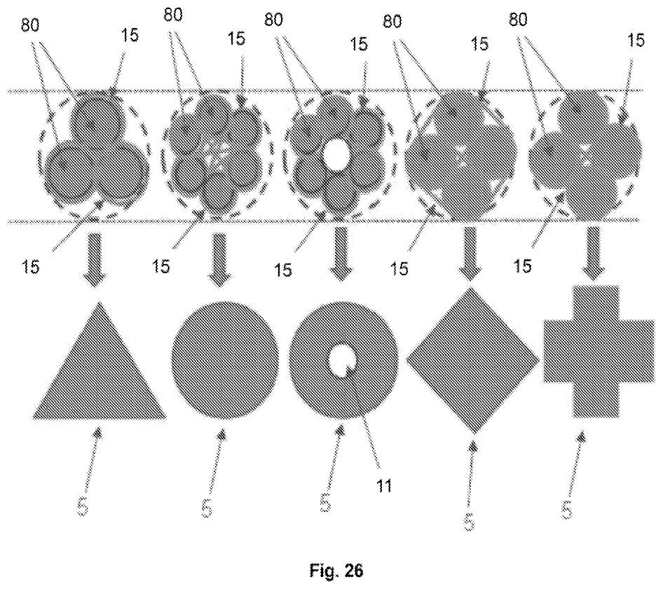

FIG. 24 illustrates two configurations of reinforcement elements 15. One configuration of reinforcement elements 15 comprise three axial fibrous bundles 80 (left) (see e.g., FIG. 26, leftmost composite implant 5). The other configuration of reinforcement elements 15 comprise six axial fibrous bundles 80 (right) (see, e.g., FIG. 26, composite implant 5 second from the left). Each of the axial fibrous bundles 80 comprise ten fibers each. The axial fibrous bundles 80 are interlocked by two sets of bias reinforcement elements 15, one set oriented −45° to axial and the other set oriented+45° to axial. The top right and top left reinforcement elements 15 were formed. The bottom left and bottom right reinforcement elements 15 were run longitudinally over a cylinder. The triangular reinforcement elements 15 (left) did not change shape and the circular reinforcement 15 (right) changes shape to a rectangular reinforcement element 15.

FIG. 25 illustrates a composite implant 5 comprising reinforcing elements 15. The reinforcement elements 15 each have triangular cross-sections. The triangular shapes can be combined, in pre-cured or thermoplastic molding processes, to create pins of different shapes. As shown, the triangular shapes combine and form a trapezoid. Three of the triangular reinforcement constructs can be combined to create a well nested composite implant 5.

FIG. 26 illustrates cross-sections of composite implants 5. The composite implants comprise axial fibrous bundles 80 and bias reinforcement elements 15. The composite implants 5 are each comprised of three, four, and six axial fibrous bundles 80, respectively. The axial fibrous bundles 80 are interlocked by bias reinforcement elements 15. The number, size and orientation of axial fibrous bundles 80 could be combined in various ways to produce different cross-sectional shapes of composite implants 5. The way in which the bias reinforcement elements 15 interlock around the axial fibrous bundles 80 may also contribute to the shape of the composite implant 5. It may be appreciated by a skilled artisan that the composite implants 5 may be combined, in any number and/or orientation to fabricate other composite implants 5. The bias reinforcement elements 15 may be configured to form a cannulation 11 (see, e.g., third from the left).

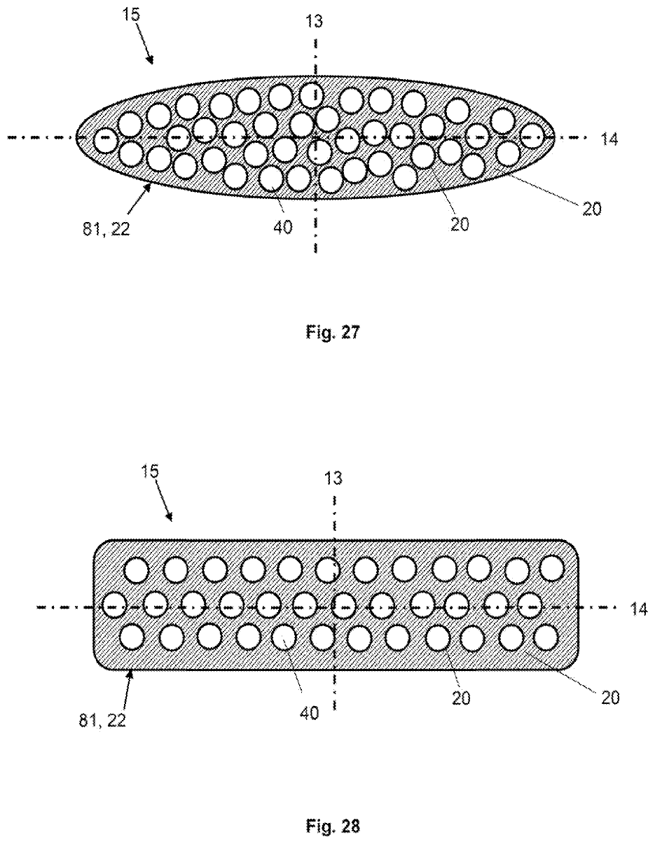

FIG. 27 and FIG. 28 illustrate cross-sections of reinforcement elements 15. The reinforcement element 15 comprise filaments 40 axially arranged therein. Each of the axially arranged filaments 40 are coated in polymeric material 20. The axially arranged reinforcement elements 15 are bounded by bias fibers 81 and a polymeric material 20 is located between the filaments 40 and within the bias fibers 81. The bias fibers 81 are aligned at an off-axis angle (+45° and)−45° to the axial filaments 40 and formed into a sheet 22. The reinforcement element 15 is defined by a longitudinal axis 14 (“major axis”) and a transverse axis 13 (“minor axis”). The major and minor axes define a cross-section aspect ratio (i.e., ratio of length to width). The reinforcement elements 15 have an elongate cross section, forming a tape. Reinforcement elements 15 having elongate cross-sections may be suitable for employment as bias fibers for winding around and/or interlacing axial reinforcement elements 15.

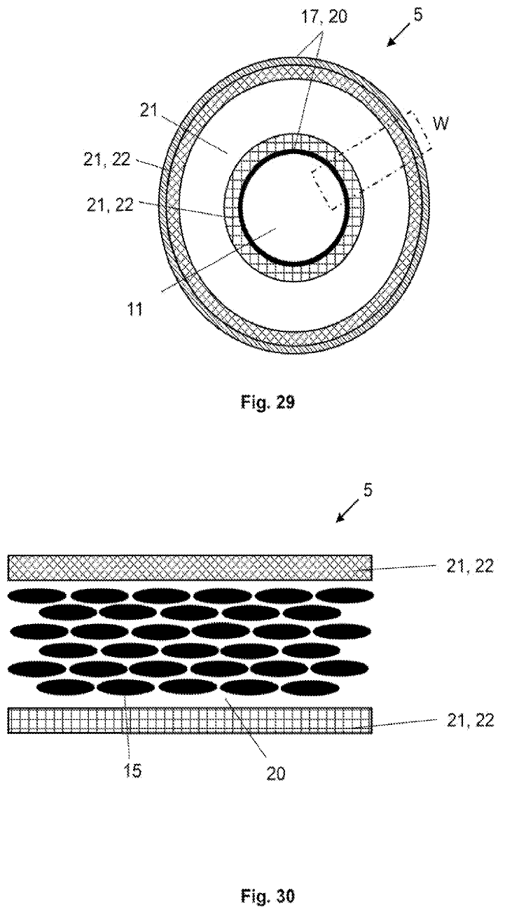

FIG. 29 illustrates a cross-section of a composite implant 5. The composite implant 5 comprises three cores 21, two outer regions 17, and a cannulation 11. The innermost core 21 comprises a reinforcement element 15 in the form of a sheet 22 (see, e.g., FIG. 10). The middle core 21 comprises a plurality of axial reinforcement elements 15 and a polymeric material 20, as shown in FIG. 30. The outermost core 21 comprises a reinforcement element 15 in the form of a sheet 22 (see, e.g., FIG. 10). One of the outer regions 17 is disposed over the outermost core. The other outer region 17 is between the innermost core 21 and the cannulation 11. The cannulation 11 is formed by working the elements of the composite implant 5 around a mandrel and thereafter removing the mandrel. The outer regions 17 comprise polymeric material 20. The polymeric material 20 may include bioglass particulates dispersed therein.

FIG. 30 illustrates a segment W of the composite implant 5, illustrated in FIG. 29. The core 21 comprises a plurality of axial reinforcement elements 15, which are nested and in a staggered alignment. A polymeric material 20 is located between the plurality of reinforcement elements 15.

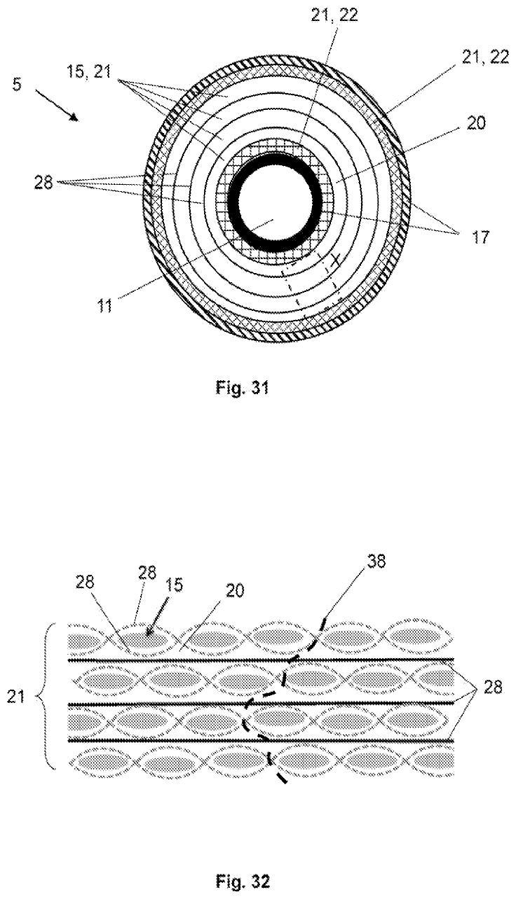

FIG. 31 illustrates a cross-section of a composite implant 5. The composite implant 5 comprises three cores 21, two outer regions 17, and a cannulation 11. The innermost core 21 comprises a reinforcement element 15 in the form of a sheet 22 (see, e.g., FIG. 10). The middle core 21 comprises four layers of a plurality of reinforcement elements 15 separated by bias reinforcement elements 28 and impregnated with a polymeric material 20, as shown in FIG. 32. The outermost core 21 comprises a reinforcement element 15 in the form of a sheet 22 (see, e.g., FIG. 10). One of the outer regions 17 is disposed over the outermost core 21. The other outer region 17 is between the innermost core 21 and the cannulation 11. The cannulation 11 is formed by working the elements of the composite implant 5 around a mandrel and thereafter removing the mandrel.

FIG. 32 illustrates segment X of the composite implant 5 illustrated in FIG. 31. The core 21 comprises four layers of a plurality of reinforcement elements 15, which are in a staggered alignment. A polymeric material 20 encapsulates the plurality of reinforcement elements 15. The layers of the plurality of reinforcement elements 15 are separated by bias reinforcement elements 28, binding each successive layer. The plurality of reinforcement elements 15 within the same layer are interlocked by two bias reinforcement elements 28. The two bias reinforcement elements 28 wind around the reinforcement elements 15 in opposing fashion. In one aspect of the present disclosure, the plurality of reinforcement elements 15 within the same layer may be interlocked by just one bias reinforcement element 28. A crack path 38 is shown extending via a tortuous path through the core layer 21. By interlocking the axial reinforcement elements 15 with bias reinforcement elements 28, a tortuous path between interfacing reinforcement elements 15 throughout the core 21 is formed. In the event of a crack originating in the composite implant 5 and translating throughout the core 21 the crack path 38 is deflected and redirected through the composite implant 5 as a result of the tortuous path.

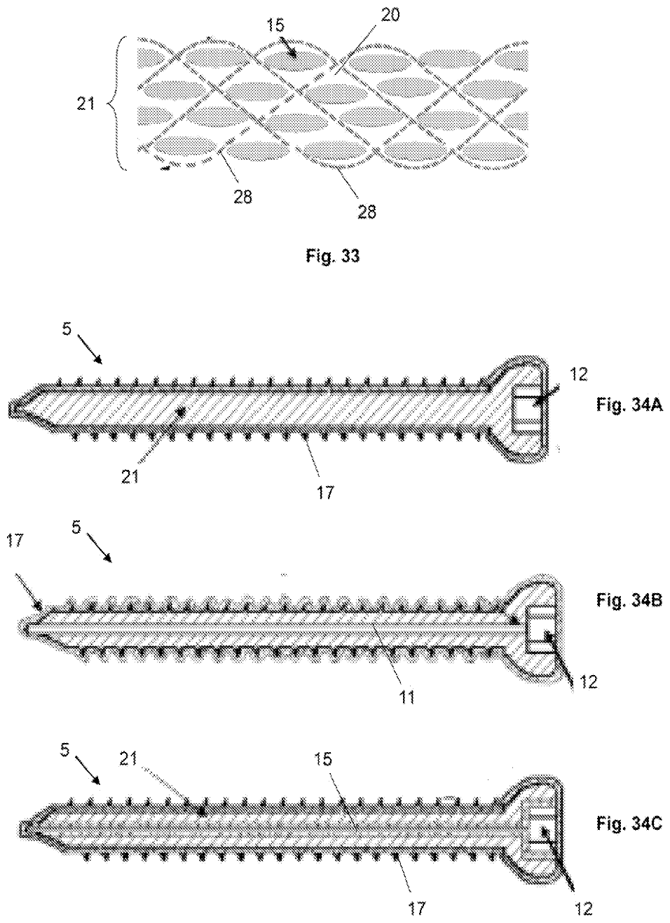

FIG. 33 illustrates segment X of the composite implant 5 illustrated in FIG. 32, according to another aspect of the present disclosure wherein the bias reinforcement elements 15 extend through multiple layers of reinforcement elements 15.

FIGS. 34A-34C illustrate composite implants 5 in the form of a screw. The composite implants 5 include a core 21 and an outer region 17. The outer region 17 (e.g., over-molded polymeric material) includes threading formed thereby. The composite implants 5 include a drive socket 12 fabricated into the core 21. As shown in FIG. 34B, a cannulation 11 extends the length of the screw. As shown in FIG. 34C, a continuous reinforcement element 15 extends longitudinally through the composite implant 15 and circumferentially around the drive socket 12, providing added protection from the head of the screw from snapping off.

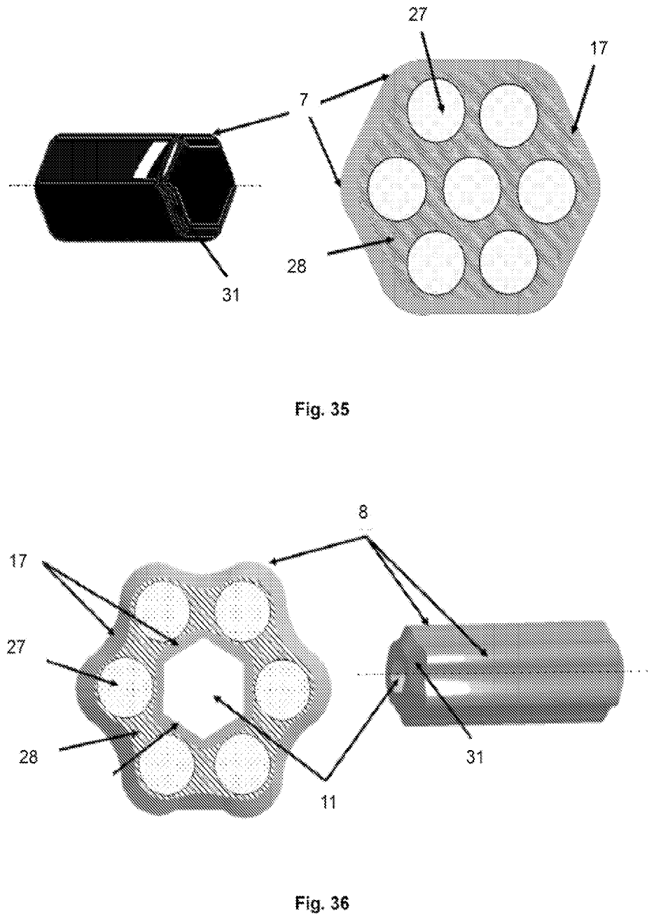

FIG. 35 is a perspective view and a cross-sectional view of a composite implant 5. The composite implant 5 is in the form of a pin. The composite implant 5 has a hexagonal cross-section. The pin is constructed with tapered ends 31. Edges 7 between facets 6 are radiused to reduce stress that would otherwise be cause by edges 7 that are sharp. The composite implant comprises seven axial reinforcement elements 27 that are interlocked with bias reinforcement elements 28. The composite implant 5 comprises an outer region 17 comprising polymeric material 20.

FIG. 36 is a perspective view and a cross-sectional view of a composite implant 5. The composite implant 5 is in the form of a pin. A cross-section of the composite implant is hexagonal and lobed. The pin is constructed with tapered ends 31. Lobes 8 are radiused to reduce stress. The composite implant 5 comprises six axial reinforcement elements 27 that are interlocked with bias reinforcement elements 28, forming a core 21. The composite implant 5 is cannulated. The cannulation has a hexagonal cross-section. The cannulation 11 and the lobes 8 extend the entire length of the pin. The composite implant 5 includes an outer region 17 surrounding the core 21 and an outer region between the core 21 and the cannulation 11. The outer regions 17 comprise polymeric material 20.

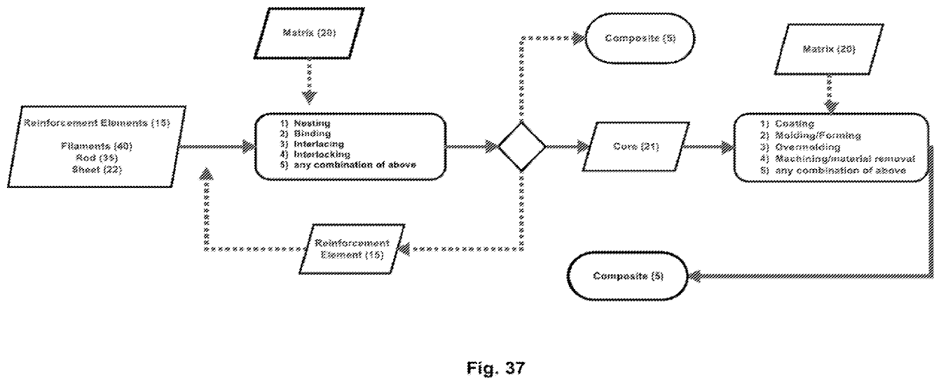

FIG. 37 illustrates a flowchart of a method according to the present disclosure.

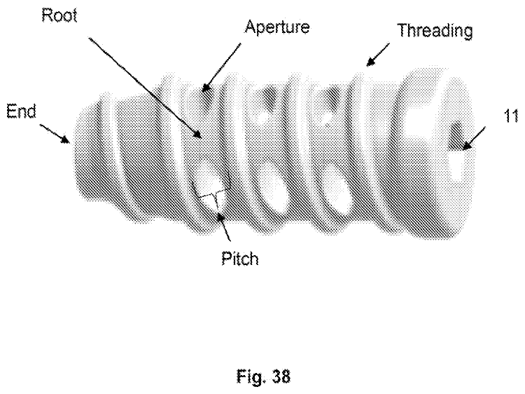

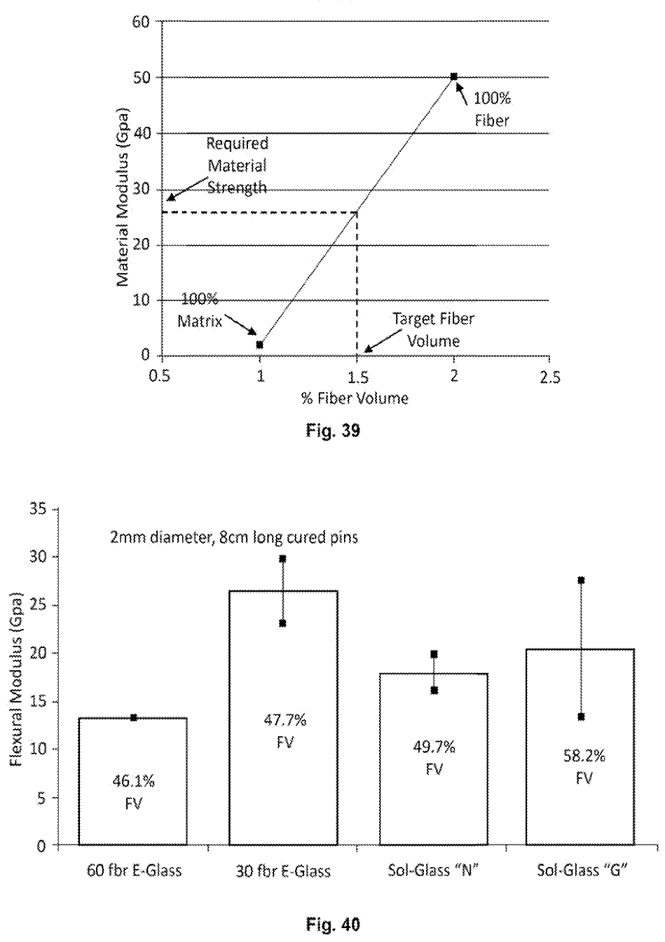

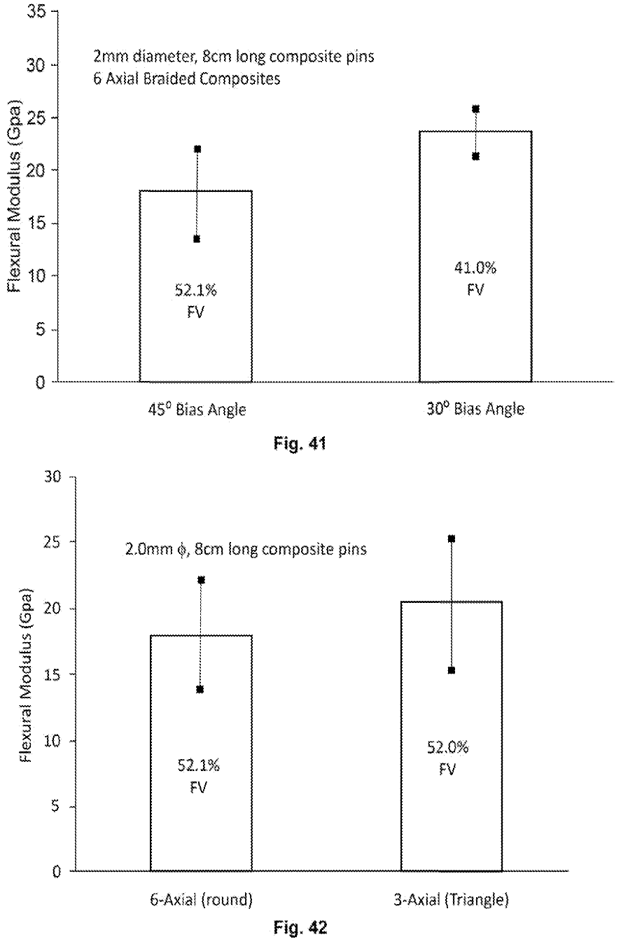

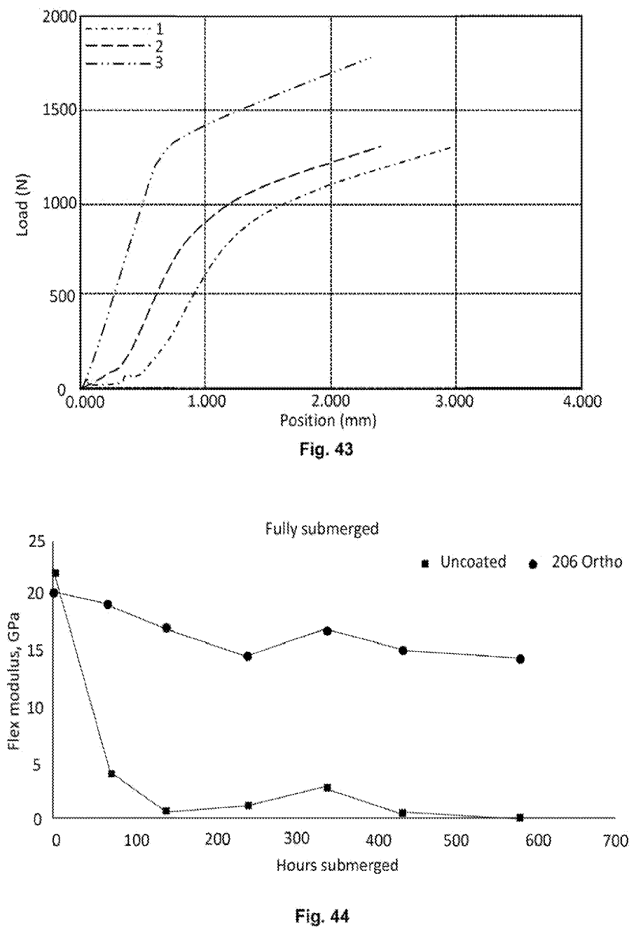

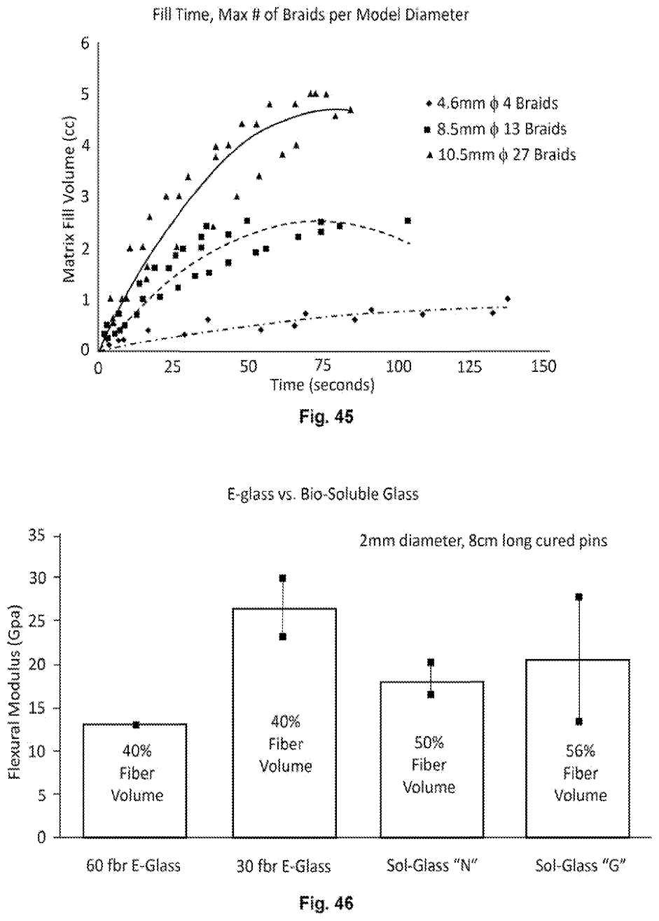

FIG. 38 illustrates a screw having threading defined by a root and a pitch. Apertures are located in the root. The screw includes two ends and cannulation 11 is exposed on one end.

### F. Illustrative Examples

The following examples include various polymers and/or fibers. It will be appreciated that the fibers may be replaced with other fibers. For example, polymeric fibers may be replaced with inorganic fibers and vice versa, or a biodegradable or bioresorbable fibers may be replaced with fibers that do not biodegrade or are not bioresorbable and vice versa. It will be appreciated that the polymer may be replaced with a different polymer. For example, a filled polymer may be replaced with an unfilled polymer or vice versa, or a biodegradable or bioresorbable polymer may be replaced with a polymer that does not biodegrade or is not bioresorbable and vice versa.

Example 1. Preparation of 50/50 prepolymer: 10.60 g polycaprolactone diol (0.02 mol), 6.00 g polycaprolactone triol (0.02 mol), both previously vacuum dried and 23.31 mL isophorone diisocyanate (0.10 mol) are stirred continuously while heating slowly to 70° C., and then stirred at 70° C. for 2 hours. The heat and stirring is stopped, and the reaction is allowed to sit at room temperature overnight. The yield is about 40 g of a clear, highly viscous material.

Example 2. Preparation of 60/40 prepolymer: 15.90 g polycaprolactone diol (0.03 mol), 6.00 g polycaprolactone triol (0.02 mol), both previously vacuum dried and 27.97 mL isophorone diisocyanate (0.13 mol) are stirred continuously while heating slowly to 70° C., and then stirred at 70° C. for 2 hours. The heat and stirring is stopped, and the reaction is allowed to sit at room temperature overnight. The yield is about 50 g of a clear, viscous material.

Example 5. Preparation of diethylenetriamine aspartic acid ester: 10.33 g diethylenetriamine (0.10 mol) and 38.74 g tert-butanol are combined, and 34.36 g diethyl maleate (0.20 mol) is added slowly. The reaction vessel is blanketed with Na and heated to 35° C. with stirring for 10 minutes. After about 120 hours at room temperature, tert-butanol is removed via rotary evaporation at 70° C. and 215-195 mbar. The yield is about 35 ml of a pale yellow slightly viscous liquid.

Example 8. Preparation of 1.5 mm diameter PLA braid: 1.5 mm braids are constructed around a core constructed of 90 ends of 75d PLLA, twisted at approximately 2 TPI. The outer sheath is constructed of 24 ends of 120d PLLA. A Steeger 48 end horizontal braider is used. The sample is repeated with glass fibers that are biodegradable and/or bioresorbable.

Example 9. Preparation of 1.5 mm diameter PLA braid with axial fibers: 1.5 mm braids are constructed around a core constructed of 90 ends of 75d PLLA, twisted at approximately 2 TPI. The outer sheath is constructed of 24 ends of 120d PLLA, and 12 axial ends of 120d PLLA. A Steeger 48 end horizontal braider is used. The sample is repeated with glass fibers that are biodegradable and/or bioresorbable.

Examples 10-14, 17, 20. Preparation of polyurethane. The components listed in the following table are mixed, transferred into a 3-ml syringe, and placed in an oven at about 37° C. to cure overnight. The cured material is removed from the syringe and cut with a diamond saw to make a specimen for mechanical testing in compression.

| | Ex. 10 | | Ex. 11 | | Ex. 12 | | Ex. 13 | | Ex. 14 | | Ex. 17 | | Ex. 20 | |
|---|---|---|---|---|---|---|---|---|---|---|---|---|---|---|
| Prepolymer of Ex. 1 | 2.6 | g | 2.6 | g | 2.6 | g | — | | — | | 5.26 | g | — | |
| Prepolymer of Ex. 2 | — | | — | | — | | 4.05 | g | 4.05 | g | — | | 8.1 | g |
| Aspartic acid ester of Ex. 5 | — | | — | | — | | — | | — | | 3.81 | g | 5.7 | g |
| tricalcium phosphate | — | | 1.0 | g | 2.48 | g | — | | 2.01 | g | — | | — | |
| polycaprolactone triol | 0.30 | g | 0.30 | g | 0.35 | g | 0.50 | g | 0.50 | g | — | | — | |
| Glycerol at 0.13% w/w | 0.10 | g | 0.10 | g | 0.10 | g | 0.15 | g | 0.15 | g | — | | | |
| dibutyltin dilaurate | | | | | | | | | | | | | — | |
| dibutyltin dilaurate | — | | — | | — | | — | | — | | — | | — | |
| Compressive stiffness | 1.1 | GPa | 1.3 | GPa | 1.8 | GPa | 1.1 | GPa | 1.5 | GPa | 0.6 | GPa | 0.7 | GPa |
| Yield strength | 56 | MPa | 63 | MPa | 71 | MPa | 53 | MPa | 69 | MPa | 29 | MPa | 20 | MPa |

Preparation of polyurethane with polypropylene braid reinforcement. A number of polypropylene braids having an inner diameter (I.e., ID) of 10 mm with triaxials are stacked one inside the other (for more than one braid) and filled with a polyurethane composition as identified in the following table. The sample is cured at 37° C. in a cylindrical mold overnight. After removing from the mold, the sample is cut with a diamond saw to make specimen for mechanical testing in compression.

Similar samples are prepared using polypropylene braids having different diameters, 10 mm ID and 5 mm ID. A number of 10 mm ID polypropylene braids with triaxials are stacked one inside the other, and a number of 5 mm ID polypropylene braids with triaxials are stacked in the same way. The smaller ID braids are placed inside the larger ID braids. The curing and following steps are the same as designated above for the samples having only the 10 mm ID polypropylene braids. The braids are concentrically nested layers of interlocked reinforcements in the form of sheets.

| | Ex. 27 | | Ex. 28 | | Ex. 29 | | Ex. 30 | | Ex. 31 | | Ex. 32 | | Ex. 33 | | Ex. 34 | |
|---|---|---|---|---|---|---|---|---|---|---|---|---|---|---|---|---|
| No. of 10 | 1 | | 2 | | 4 | | 4 | | 1 | | 2 | | 4 | | 4 | |
| mm braids | | | | | | | | | | | | | | | | |
| No. of 5 | | | | | | | 3 | | | | | | | | 3 | |
| mm braids | | | | | | | | | | | | | | | | |
| Filling | PU from | | PU from | | PU from | | PU from | | PU from | | PU from | | PU from | | PU from | |
| | Ex. 13 | | Ex. 13 | | Ex. 13 | | Ex. 13 | | Ex. 14 | | Ex. 14 | | Ex. 14 | | Ex. 14 | |
| Compressive | 1.3 | GPa | 1.0 | GPa | 1.3 | GPa | 1.2 | GPa | 1.0 | GPa | 1.7 | GPa | 2.0 | GPa | 1.7 | GPa |
| stiffness | | | | | | | | | | | | | | | | |
| Yield | 69 | MPa | 44 | MPa | 69 | MPa | 63 | MPa | 53 | MPa | 75 | MPa | 66 | MPa | 70 | MPa |
| strength | | | | | | | | | | | | | | | | |

*PU = polyurethane

Example 35. Preparation of Polyurethane with braid reinforcement: One 1.5 mm ID PLA braid with axials is loaded into a 2 mm ID tube and filled with the polyurethane composition of Example 13 after degassing with no DBDL. The sample is cured at 70° C. for two days. The cured sample is removed from the tubing and tested for mechanical properties using a three-point bending test.

Example 36. Preparation of Polyurethane with braid reinforcement: One 1.5 mm ID PLA braid without axials is loaded into a 2 mm ID tube and filled with the polyurethane composition from Example 13 after degassing with no DBDL. The sample is cured at 70° C. for two days. The cured sample is removed from the tubing and tested for mechanical properties using a three-point bending test.

Example 37. Preparation of Polyurethane with braid reinforcement: One 5 mm ID PLA braid without axials is loaded into a 5 mm ID tube and filled with the polyurethane composition from Example 13 after degassing with no DBDL. The sample is cured at 70° C. for two days. The sample is removed from the tubing for three-point bending test. Three-point bend testing shows that the material has a stiffness of 1.2 GPa and a yield strength of 39 MPa.

Example 38. Preparation of Polyurethane with braid reinforcement: One 10 mm ID PLA braid without axials is filled with the polyurethane composition from Example 13 after degassing with no DBDL. The sample is cured at 70° C. in a cylindrical mold for two days. The sample is removed from the mold and cut using a diamond saw to make a compression test piece. Compression testing shows that the material has a compressive stiffness of 0.8 GPa and a yield strength of 39 MPa.

Example 39. Preparation of Polyurethane with braid reinforcement: One 7 mm ID PLA braid without axials is placed inside of a 10 mm ID PLA braid without axials and filled with the polyurethane composition from Example 13 after degassing with no DBDL. The sample is cured at 70° C. in a cylindrical mold for two days. The cured sample is removed from the syringe and cut using a diamond saw to make a compression test piece. Compression testing shows that the material has a compressive stiffness of 0.5 GPa and a yield strength of 27 MPa.

Example 40. Preparation of Polyurethane with braid reinforcement: One 5 mm ID PLA braid without axials is placed inside of a 7 mm ID PLA braid without axials and both braids are placed inside of a 10 mm ID PLA braid without axials, and the entire stack is filled with the polyurethane composition from Example 13 after degassing with no DBDL. The sample is cured at 70° C. in a cylindrical mold for two days. The sample is removed from the mold and cut using a diamond saw to make a compression test piece. Compression testing shows that the material had a compressive stiffness of 0.8 GPa and a yield strength of 39 MPa.

### Examples 41-50 Glass Braid Composites

Preparation of 60/40 prepolymer having a molar ratio of diol to triol of about 60:40. 15.90 g polycaprolactone diol (0.03 mol), 6.00 g polycaprolactone triol (0.02 mol), both previously vacuum dried and 27.97 ml isophorone diisocyanate (0.13 mol) are stirred continuously while heating slowly to 70° C., and then stirred at 70° C. for 2 hours. The heat and stirring are stopped, and the reaction is allowed to sit at room temperature overnight yielding ˜50 g of clear viscous material. It will be appreciated that the molar ratio of the diols to triols may be changed and/or additional materials (e.g., having four, five, or more OH groups may be employed). Preferably, the amount of monomers having three or more OH groups is sufficiently high so that a cross-linked network can be formed.

Textile containing composites are prepared using bundles of fibers that are braided with bias fiber elements. The bias fiber element has an elongated cross-section, for example about 1 fiber in a thickness direction and 2 or more fibers in a width direction. The textile includes 3 bundles of fibers having axially aligned fibers oriented in the axial direction of the textile. The 3 fibrous bundles are bound by bias fibers. It will be appreciated that more or fewer bundles of fibers may be used in preparing a textile and the number of bundles preferably is from 3 to 20, more preferably 3 to 10. The fibers used in the bundle of fibers and in the bias fibers are glass fibers. The glass fiber is E-glass. However, other inorganic fibers, or glass fibers may be employed. For example, the fibers may be formed of a biodegradable or bioresorbable material, such as a biodegradable and/or bioresorbable glass. A volume ratio of the fibers in the axial fibers (in the bundles of fibers) to fibers in the bias fiber elements is about 1:1. It will be appreciated that a higher or lower weight ratio may be employed. The volume ratio may be about 0.25:1 or more, about 0.5:1 or more, about 0.75:1 or more, about 0.90 or more, about of 1:1 or more, about 1.2:1 or more, about 1.5:1 or more, or about 2.0:1 or more. The volume ratio may be about 4:1 or less, about 3:1 or less, about 2:1 or more, about 1.5:1 or less, about 1:1 or less, about 0.80 or less, about 0.60:1 or less, or about 0.5:1 or less. The bias fiber may be used individually or may be provided as a group of two more fibers. The bias fibers are orientated at +/−45 degrees relative to the axial fibers in the bundles of fibers. It will be appreciated that other orientations may be employed. Preferably the bias fibers are oriented at angles from about +/−10 degrees to about +/−90 degrees, more preferably from about +/−20 degrees to about +/−70 degrees relative to the axial direction. The resulting construct is a textile having a predominantly triangular cross-section having a diameter of about 1.9 to 2.2 mm and a length of about 80 mm. A composite is prepared from a single textile construct, by placing the textile in a PTFE tube and filling with a polymerizable composition. These examples, employ a polyurethane formulation, as shown in the table below. However, it will be appreciated that other polymeric materials and/or polymerizable resins may be employed. The polyurethane formulation is injected into the PTFE tube using both injection pressure and vacuum suction to produce a substantially void free construction with approximately 50% glass by volume. The construction is cured at 70 degrees C. in a tight-fitting stainless-steel tube and cut from the PTFE tube. The resulting composite is in the form of a composite pin. The pin is removed and subjected to mechanical testing. For comparison, pins are also prepared using only the polyurethane formulation, without any fibers and tested for mechanical properties.

Examples 41-50 use commercially available polyester polyols from King Industries (Kflex series), Perstorp (Capa) and Invista (Terin), all are known to hydrolytically breakdown over a period of time under ambient aqueous environments. Capa 4101 is a polyol (tetrols) having a molecular weight of about 1,000, a melting range of about 10-20° C., an OH value of about 218, and a viscosity about 260 mPas at 60° C. Capa 4800 is a polyol (tetrol) having a molecular weight of about 8,000, a viscosity of about 40-50° C., and a viscosity of about 4,700 mPas at 60° C. The isocyanate prepolymer is the same as described in Example 2 with the polycaprolactone diol and triol being sourced from Perstorp. The polyols are precombined and allowed to stand to remove an air entrainment. The isocyanate prepolymer is combined with the polyol blend at the ratios shown in the table below. The amount of isocyanate prepolymer and the amount of the polyol blend is determined so that there is generally stoichiometry in the reactive groups (e.g., hydroxy value and isocyanate value). It will be appreciated that other ratios (e.g., non-stoichometric ratios) may be employed). For example, the ratio of hydroxy groups to isocyanate groups may be from about 0.66 to about 1.50, about 0.66 to about 1.00, about 1.00 to about 1.50, about 0.90 to about 1.11, or about 0.95 to about 1.06. The isocyanate prepolymer/polyol mixture is degassed before injecting into the tubes to avoid air entrainment. The samples are cured at 70 degrees C. for 48 hours and then conditioned under ambient conditions before being tested for flexural strength.

| Examples | 41 | 42 | 43 | 44 | 45 | 46 | 47 | 48 | 149 | 50 |
|---|---|---|---|---|---|---|---|---|---|---|
| Polyol, parts by wt | | | | | | | | | | |
| Kflex 366, pbw | 60 | | | | 60 | | | | | |
| Kflex 307, pbw | | 60 | | | | 60 | | | | |
| Kflex XM 337, pbw | | | 60 | | | | 60 | | | |
| Kflex 148, pbw | | | | | | | | | 60 | 60 |
| Terin 168G, pbw | | | | 60 | | | | 60 | | |
| Capa 4101. pbw | 30 | 30 | 30 | 30 | | | | | 30 | |
| Capa 4800, pbw | | | | | 30 | 30 | 30 | 30 | | 30 |
| Glycerol + 10% | 10 | 10 | 10 | 10 | 10 | 10 | 10 | 10 | 10 | 10 |
| DBTL, pbw | | | | | | | | | | |
| Total, pbw | 100 | 100 | 100 | 100 | 100 | 100 | 100 | 100 | 100 | 100 |
| Isocyanate | 256 | 210 | 243 | 221 | 220 | 173 | 211 | 184 | 245 | 208 |
| prepolymer, pbw | | | | | | | | | | |
| Gel time (min) at | 13 | 18 | 11 | 17 | 19 | 10 | 8 | 18 | 20 | 27 |
| about 23° C. | | | | | | | | | | |
| Properties of cured | | | | | | | | | | |
| resin | | | | | | | | | | |
| Modulus (GPa) | 1.4 | 0.8 | 1.9 | 0.7 | 2.0 | 1.6 | 2.3 | 1.6 | 1.5 | 2.2 |
| Failure mode | ductile | ductile | ductile | ductile | ductile | ductile | ductile | ductile | ductile | ductile |
| Properties of cured | | | | | | | | | | |
| composite | | | | | | | | | | |
| Flex Modulus | 15.6 | 12.9 | 20.3 | 16.2 | 18.1 | 16.4 | 16.6 | 11.7 | 17.3 | 14.8 |
| (GPa) | | | | | | | | | | |
| Failure mode | ductile | slip | break | slip | slip | ductile | break | ductile | ductile | ductile |

The data in the table above shows the effect of the polyol type and composition on gel time and flexural modulus of the cured resin and the ability to tailor performance. Similarly, the incorporation of the glass reinforcement showed substantial increases in flexural modulus by 10- to 15-fold in most cases still maintaining a ductile failure mode. This increase is substantially higher than the change in properties seen in prior examples with polypropylene and PLA fiber reinforcements.

Examples 51-60. Using the same procedure as described in Examples 41-50, a series of cured polyurethane compositions are evaluated both as a neat polymer and as a composite element including 3 fibrous bundles braided with glass bias fiber elements. The mechanical properties for each polyurethane is shown in the table below, for the neat polyurethane and the composite element including the textile constructions previously described for Examples 41-50.

| Examples | 51 | 52 | 53 | 54 | 55 | 56 | 57 | 58 | 59 | 60 |
|---|---|---|---|---|---|---|---|---|---|---|
| Polyol | | | | | | | | | | |
| Capa 2504 | 45 | 35 | 30 | 35 | 30 | 45 | 35 | 30 | 35 | 30 |
| EG/Dilactide (1:2 | 45 | 35 | 30 | 35 | 30 | | | | | |
| molar ratio) | | | | | | | | | | |
| EG/Dilactide (1:4 | | | | | | 45 | 35 | 30 | 35 | 30 |
| molar ratio) | | | | | | | | | | |
| Capa 4101 | 0 | 20 | 30 | | | 0 | 20 | 30 | | |
| Capa 4800 | | | | 20 | 30 | | | | 20 | 30 |
| Glycerol + 10% | 10 | 10 | 10 | 10 | 10 | 10 | 10 | 10 | 10 | 10 |
| DBTL | | | | | | | | | | |
| Total | 100 | 100 | 100 | 100 | 100 | 100 | 100 | 100 | 100 | 100 |
| Isocyanate | 230 | 233 | 234 | 209 | 196 | 205 | 213 | 217 | 189 | 180 |
| prepolymer | | | | | | | | | | |
| Gel time (min) rt | 81 | 96 | 124 | 124 | 88 | 32 | 34 | 22 | 34 | 22 |
| Properties of Cured | | | | | | | | | | |
| Resin | | | | | | | | | | |
| Flex Modulus | 2.9 | 2.4 | 1.3 | 1.6 | 1.3 | 2.6 | 2.3 | 2.6 | 2.5 | 1.5 |
| (GPa) | | | | | | | | | | |
| Failure mode | ductile | ductile | ductile | ductile | ductile | ductile | ductile | ductile | ductile | ductile |
| Properties of Cured | | | | | | | | | | |
| Composite | | | | | | | | | | |
| Flex Modulus | 23.6 | 17.3 | 18.0 | 14.9 | 16.7 | 15.5 | 13.4 | 15.8 | 8.2 | 2.8 |
| (GPa) | | | | | | | | | | |
| Failure mode | ductile | slip | break | slip | slip | ductile | break | ductile | ductile | ductile |

Examples 51-60 show the effect of a different type of polyester polyol, in this case made from the reaction of ethylene glycol and DL dilactide using the method below:

Preparation of high molecular weight DL-lactide: 5.15 grams of DL-lactide monomer is added to 0.31 grams ethylene glycol and 0.0016 grams stannous 2-ethylhexanoate and heated to 120° C. for 24 hours to produce a clear viscous fluid.

Preparation of DL-lactide diol: 7.19 grams of DL-lactide monomer is added to 1.56 grams ethylene glycol and 0.0029 grams stannous 2-ethylhexanoate. The mixture is heated to 120° C. for 24 hours to produce a clear slightly viscous fluid.

Preparation of low DL-lactide diol: 7.21 grams of DL-lactide monomer is added to 3.10 grams ethylene glycol and 0.0030 grams stannous 2-ethylhexanoate. The mixture heated to 120° C. for 24 hours to produce a clear low viscosity fluid.

By selecting the type and amount of dilactide polyol, the flexural modulus of the cured resin may be changed from 1.3 GPa to 2.9 GPa which is very significant.

Examples 41-50 also show that the flexural modulus of the neat polymer may be increased from 2.9 GPa to about 23.6 GPa for the composite element including a fibrous composite (e.g., braided textile). Thus, it is shown that physical properties of the implant material and/or components of the implant may be tailored. In particular, it is possible to obtain a structure (e.g., a composite element) that is both ductile and stiff. The high stiffness of the material is particularly surprising considering the generally low concentration of fibers that are aligned in the axial direction.

Example 61. A liquid polyurethane polymerizable resin material for a polymeric matrix is prepared as follows: 4.05 grams of the prepolymer of Examples 41-50 is mixed with 2.01 grams of tricalcium phosphate and 0.50 grams of polycaprolactone triol and 0.15 grams of glycerol 0.13% w/w dibutyltin dilaurate. 3 mm proximal entry holes and 3 mm mid-shaft lesions are created in 5 New Zealand White rabbits. A braided textile construct is compressed into a sheath and delivered through a catheter having an inner diameter of approximately 0.080 inch. The braided textile construct is inserted though the proximal entry and positioned across the mid-shaft lesion. The liquid polyurethane polymerizable resin material is injected within and around the braided construct using a catheter with a distal portal. The polymerizable resin material reacts to form a cross-linked polymer. During the cure, a significant amount of foaming occurs, due to the contact of the polymerizable resin material with the water in the blood. The resin cured in situ to form a foamed polymeric matrix, resulting in an internal composite splint. In some instances, the matrix expanded and/or flowed into the fracture gap. After 6 weeks, lesions demonstrate healing except where the matrix enters the fracture gap. In all cases, no abnormal bony reactions or infections occurs. This demonstrates that a modular splint can be constructed through a minimally invasive entry and will not interfere with normal bone healing using an engineered matrix reinforcement filled in series with a matrix material. It also highlights the requirement for a containment system to maintain or span the fracture gap as well and/or to contain the curing of the polymer and direct expansion of the matrix and/or to isolate the polymerizable resin from fluids in and around a bone.

Example 62. A textile based composite material is prepared using the braided textile construct describe in examples 41-50 except the glass fibers are replaced with soluble phosphate glass fibers having a sizing. The composite has a diameter of about 1.9 to 2.0 mm and a length of about 80 mm. Multiple strands of the composites is placed in a PTFE tube. The mixture of prepolymers of Example 51 is degassed and injected into the PTFE tube using both injection pressure and vacuum suction over many hours to produce predominantly void free composites which are cured at 70° C. The PTFE tube is cut, and the cured composite, in the form of a pin is removed and subjected to mechanical testing. The composite has a flexural modulus of 37 GPa and the concentration of fibers is about 71% by volume. This demonstrates that the use of a bioabsorbable glass as the reinforcements from this invention produces results similar to the aforementioned e-glass samples and that the invention can produce composites with greater than bone-like physical properties. It also demonstrated the long length of time required to fill and wet-out non-textile engineered uniaxial directed bundles with high fiber volume.

This can also be seen in example 98 and 62 where nested bundles of reinforcing elements at approximately embedded in a matrix at approximately 71% fiber volume to create a pin or rod with a 2 mm diameter.

### Examples 63A

A series of examples are prepared to see if it is at all possible to develop a composite having both high stiffness, high strength and ductility. Some of the variables that are explored are 1) separating the uniaxial filaments into multiple bundles, 2) the number of filaments in a single bundle; 3) adding a twist to the filaments in a bundle; 4) evaluating different levels of twist. The filament bundles are processed through a cross-head extruder and embedded in a degradable polymer or a polymerizable material to form a polymer matrix. In one set A, the matrix is a thermoplastic polymer and in set B, the matrix is a thermoset polymer. The matrix is biodegradable and/or bioresorbable. The surface of the resulting rod is a matrix layer and the lateral surfaces of the fibers are not exposed. The diameter of the filaments ranged from 9 μm to 35 μm. The fiber is a glass having a tensile modulus of about 75 GPa and a tensile strength of about 2.0 to 2.2 GPa, and a density of about 2.50-2.67. The number of filaments in a fiber bundle ranged from 15 to about 12000. Fibers having no twist, fibers having a twist of 0.3, 0.5, 0.7, 1.3, 3.5 turns per inch are used in the fiber bundles. Before coating with polymer, the fibers having less than 2000 filaments are flexible and have a bending radius of less than 2 cm and can be wound around a spool having a 2 cm outer diameter. The composite formed by coating the multiple fiber bundles is a rod having a diameter of about 2 mm. The fiber volume of the rod is about 55% for the thermoplastic based samples and about 60% for the thermoset based samples. The rods are tested using flexural testing (3-point bend test). The size of each fiber bundles and the twist rate of the filaments in the bundles both affected that flexural strength, the stiffness (e.g., flexural modulus), the ductility and the failure mode of the rod. The strength was most greatly affected by including a twist to the fibers. Without a twist, the flexural strength is about 280-290 MPa. By adding a twist, it is possible to increase the flexural strength to about 400 to 700 MPa, or even more. By example, a rod made with fiber bundles having 400 filaments per bundle had a flexular modulus of about 27 GPa, and the fiber bundles having about 200 filaments had a flexural modulus of about 13 GPa. The best properties are seen with a twist of about 0.3 to 1.5 turns per inch, preferably 0.5 to 1.3 turns per inch. The rods containing large uniaxial fiber bundles are brittle and have a strain at failure of 2% or less. By using smaller fiber bundles the failure mode is changed from a brittle failure to a ductile failure and the strain at failure is increased to greater than 2%. Preferably the number of filaments in a bundle is 15 to 800, more preferably 30 to 600, and most preferably 50 to 400. The rods based on the smaller fiber bundles resulted in a more homogeneous structure, with about 2% lower fiber volume, yet still having about the same stiffness/modulus compared to the large bundles. The most homogeneous structure is seen with small fiber bundles having a twist. The rods are evaluated for in vitro degradation in simulated body fluid at 37° C. for 1 to 52 weeks. The samples with the small fiber bundles had a lower degradation rate and can be used to control degradation rate. It is also determined that the twist should be approximately balanced for best processability, meaning the number of S and Z twists should be about the same.

Example 63B. Bound bundles of fibers. The samples of Example 63A are repeat except a bias fiber element is added to the outside of the multiple bundles. In this series the fibers have a twist of 0.7 twists per inch and the number of filaments per bundle is about 204 or about 400. The bias fiber element is a braid that surrounds the uniaxial core formed from the fiber bundles. The number fibers in the core is reduced so that the total diameter of the rod is maintained at 2 mm. The bias fiber elements used in the braid are angled at either 30°, 45°, and 55° relative to the axial direction. The ratio of the filaments in the core to the filaments in the braid is about 1:1. The total fiber volume is maintained at about 52%. By using the bias fibers elements in forming a braid, the ductility and failure mode is maintained and torsional testing shows an increase of about 50% or more in both strength and modulus. The flexural modulus of the composite element (e.g., rod) can be decreased with a lower bias angle and increased with a higher bias angle.

Example 63C. Interlocked and bound bundles of fibers grouped in multiple nested bundles. Example 63B is repeated except the core is replaced by multiple axial fibrous bundles which are interlocked together by the bias fiber elements instead of using the outer braiding. A ratio of filaments in the axial fibrous bundles and the bias fiber elements is about 1:1. The number of axial fiber bundles is 3 or 6. The filaments are divided equally into the 3 or 6 axial fiber bundles. The resulting rods have a diameter of about 2 mm. The fiber volume is about 52%. The mechanical testing shows that the property improvements discussed above are generally maintained. By using the interlocked bias fiber elements, the flexural modulus is also increased and is nearly as high as the large bundles that were evaluated in Example 63A. The 2 mm rods are then packed into a 7.5 mm tube. About 11 to 12 rods are packed into the tube and the tube is filled with the same matrix polymer as used in the rods. For comparison, sample where only uniaxial fibers and matrix material is placed in the tube. The concentration of fibers in the 7.5 mm rods is about 50 volume percent. A 7.5 mm rod of PEEK is also evaluated for comparison. Compression testing shows of the samples show the following results.

Properties of 7.5 mm rods

| | 12 rods each | | | |
|---|---|---|---|---|
| | having 3 | 12 rods each | | |
| | interlocked | having 6 | | |
| | fiber bundles | interlocked | Uniaxial | |
| | and a diameter | fiber bundles | fibers | |
| | of 2 mm joined | and a | joined | |
| | by matrix | diameter | by matrix | |
| | material | of 2 mm | material | PEEK |
| Ultimate | 200 | 230 | 255 | 120 |
| compressive | | | | |
| stress, MPa | | | | |
| Compressive | 6% | 6% | <2% | 8 to 9% |
| Strain at Peak | | | | |
| Stress, % | | | | |
| Torsional, MPa | 560 | | | 240 |

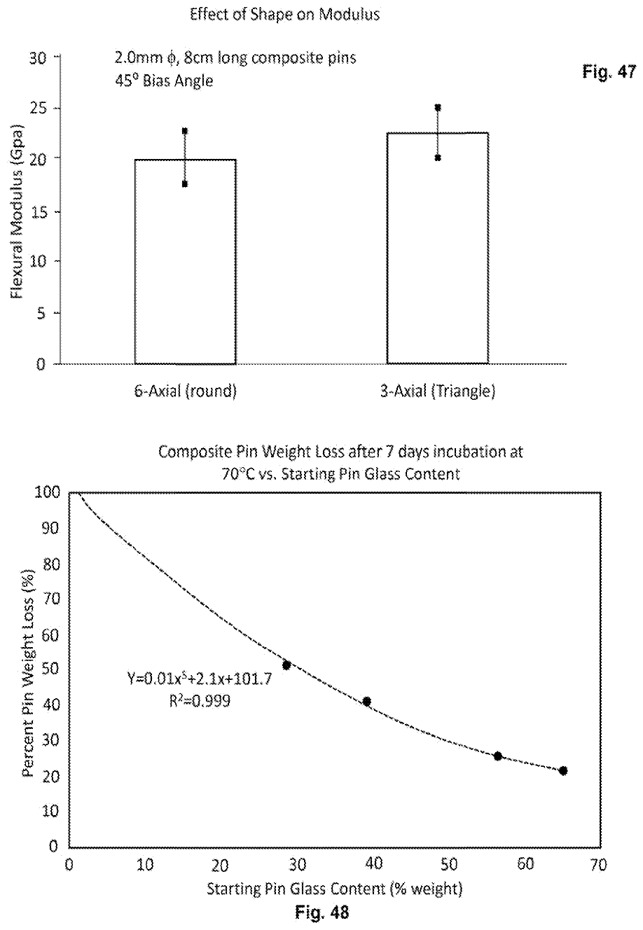

Example 64A. Glass fibers from AGY (60 fbr glass above) and PPG (30 fbr glass above). Each glass fiber has a different fiber diameter. These are compared to two types of bio-soluble glasses axially orientated within a composite using the same polyurethane matrix from Example 51 and using the same methods as described in Example 62. A comparison of flexural modulus is shown in FIG. 47 and demonstrates that the smaller “60 fiber” glass (when adjusted for fiber volume) is a good surrogate for bio-soluble glass fibers and therefore justifies the use in Examples 41-60 and those that follow.

Example 64B. Glass fibers from AGY (60 fbr glass above) and PPG (30 fbr glass above) are used as fibrous bundles and bias fiber elements for preparing a composite 2 mm pins using the same polyurethane matrix and method of construction described in Example 64A. The fibers have a sizing for improved adhesion to the matrix material. The fibers differed in two manners, the diameter of one fiber is twice that to the other (filament diameters are the same for both) however the fiber volume is kept consistent, and there is a coating difference between the two (proprietary to each e-glass manufacturer). A comparison of flexural modulus is shown in FIG. 47 with a marked difference in modulus between the two composite rods. The results demonstrate that the axial strength may be dramatically increased by through the use of an appropriate fiber coating used to compatibilize the matrix to the reinforcing elements.

Example 65. Textile glass braids are prepared having 6 axial fibrous bundles (predominantly circular cross-section) bound by bias fiber elements. The ratio of the volume of glass in the axial fibrous bundles to the volume of glass in the bias fiber elements is about 1:1. In Example 65-1, the bias fiber elements are orientated at +/−45° to the axial fibrous bundles. In Example 65-2, the bias fiber elements are orientated at +/−30° to the axial fibrous bundles. 2 mm composite elements in the form of pins or rods are built using the same polyurethane matrix and method of construction described in Example 64A. The total fiber concentration in Example 65-1 and 65-2 is about 52.1 volume percent and about 41.0 volume percent, respectively. The flexural modulus of Examples 65-1 and 65-2 is shown in FIG. 48. These results demonstrating that the stiffness of the structure of the composite elements be increased significantly by changing the angle of the bias fiber elements in the braided structure. The flexural modulus of the composite element (e.g., the pin) can be decreased with a lower angle and increased with a higher angle.

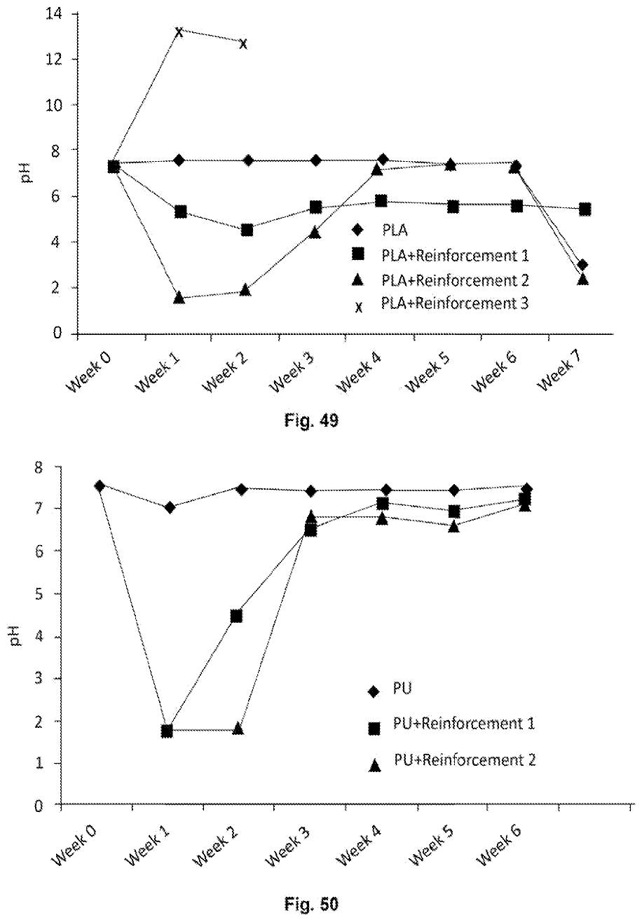

Example 66. Two samples of textile glass braids are prepared. In Example 66-1, the textile includes 6 axial fibrous bundles. In Example 66-2, the textile includes 3 axial fibrous bundles. Each of the fibrous bundles has a predominantly having a circular cross-section. In both Examples 66-1 and 66-2, the fibrous bundles are bound by bias fiber elements orientated at +/−45° to the axial bundles. The ratio of the volume of fibers in the fibrous bundles to the volume of fibers in the bias fiber elements is about 1:1. The textiles used in Examples 66-1 and 66-2 have about the same volume of fiber per unit length. The textile is then used to make 2 mm diameter fibrous composite in the form of cylindrical pins using the same polyurethane matrix and method of construction described in Example 64A. The flexural modulus of the fibrous composites pins is shown in FIG. 49. Examples 66-1 and 66-2 both have about 52 volume percent total fiber content. The two fibrous composites have about the same flexural modulus.

Example 67. Textile glass braids are prepared having 6 axial fibrous bundles bound by bias fiber elements in a glass content ratio of approximately 1:1 (total volume of fibers in the fibrous bundles to total volume of fibers in the bias fiber elements). The bias fiber elements are orientated at +/−45 degrees to the fibrous bundles. The resulting textile has a predominantly circular cross-section. The fiber by weight per unit length braid is designed to be approximately the same as the predominantly triangular cross-section E glass braided textiles from Examples 41-50. Multiple sections of this braid textile including fibrous bundles are packed lengthwise into a PTFE tube having an inner diameter of about 7.5 mm. The braided textiles of Examples 41-50 are similarly fit into a 7.5 mm ID PTFE tube. Triplicate samples are prepared for each material. With the triangular shaped textiles or fibrous composites, 12 lengths of the material could be fit into the tube. of the predominantly triangular cross-section braids could fit parallel in the tube (for a fiber content of about 49.4% by volume). With the circular-shaped cross-section, only 11 lengths could be packed into the tube, resulting in a total fiber volume of about 47.0%. By using a textile or pin geometry that more intimately nests, it is possible to increase the final fiber concentration (FV) of a composite. material that nests. This confirms the importance of the shape of the various scalable components (e.g., fibrous bundles, fibrous composites, and composite elements) for achieving nesting and the ability to increase the fiber volume of the final composite part, such as an implant. The concept of nesting and fit is demonstrated in FIG. 24 and FIG. 25.

Example 68. The flexural modulus for Example 67 shows no significant difference between the circular and triangular shape of the textile, despite the inclusion of more reinforcement rods into the composite. The large number of reinforcement rods makes the difference in mechanical properties small, so the ratio of standard deviation to average value (expressed in %) is used to compare the variability. The triangular vs. circular cross section braids come off of the manufacturing storage roll differently. The triangular braids maintain a shape, while the circular ones come off of the roll in a rectangular shape. The rectangular shape acts to promote intra-braid nesting, creating good axially oriented columns (better bending). The variability in bending performance slightly favors the rectangle/circular design (5% vs. 9% variability). However, in torsion, the triangular shapes are much less variable than the rectangular/circular (2% vs. 13%). Showing that in torsional resistance, the triangular shapes inter-nest much better.

The shapes are also important in function. The long triangular shapes hold a vertical posture better in a less hardened, more flexible (non-composite) state, therefore will be better for insertion into long straight bones such as the humerus, tibia or femur. The rectangular shapes bend better around curves in bones such as the clavical without buckling.

Example 69. The value of the braided reinforcement construct is further demonstrated when compared to uni-axial constructs. Uni-axial constructs are made with the same fibers and the same methods as those in Example 67 except the braided textile is replaced with the uni-axial construct, having no bias fibers. The resulting composite construct has as similar fiber volumes (45% FV vs. 49.4% FV—triangular constructs and 47% FV for circular constructs). The performance in bending is better than the braids (all fibers are axially oriented), however the results had significantly higher variability (17% compared to 5 or 9%) and took much longer to fill with resin and had spots within the construct that are not completely wet-out after hours of filling. In torsion, the uni-axial composite variability is similar (7% compared to 2%, triangular constructs or 13% circular constructs) but the performance is 29% lower than the braided constructs. This performance is expected since the braided textile constructs (both circular and triangular) have 50% of the fiber volume contributing 50% of its strength (45° bias angles) to non-axial forces. This is an example of reduced filling variability using braided constructs. The bias fiber elements may provide channels for the flow of liquid into the fiber structure. Flow may also be accelerated by engineering in of hydrostatic force inducing elements that pull matrix through the full construct, such as by wicking. It also demonstrates the advantage of being able to variably assign reinforcement to different directions of support. In addition, the constructs are simple, loadable structures, wherein uni-axial constructs would be very difficult to load without significant coating (that would reduce wet-out and/or fiber volume) to stiffen the components.

Example 70. An example is depicted in FIG. 25 of how multiple triangular reinforcement shapes (e.g., triangular shaped fibrous bundles or triangular shaped composite elements) such as those depicted in Example 68 can be combined, in pre-cured thermoset or thermoplastic molding processes, to create pins of different shapes as well. Three of the triangular reinforcement constructs from Example 68 can be combined to create a well nested final implant of unique shapes. It will be appreciated that this nesting may be applied to fibrous bundles, fibrous composites, composite elements, and textiles including both axial fiber bundles and bias fiber elements, for scaling to larger sized components. In each step of scaling, additional bias fiber elements may be provided and/or an additional coating of a polymeric material may be provided. For example, the additional fiber elements may be an additional braid or other weave that goes around the multiples of the smaller component or between the smaller components. The additional coating may join together the smaller components and/or may coat or infiltrate the additional braid.

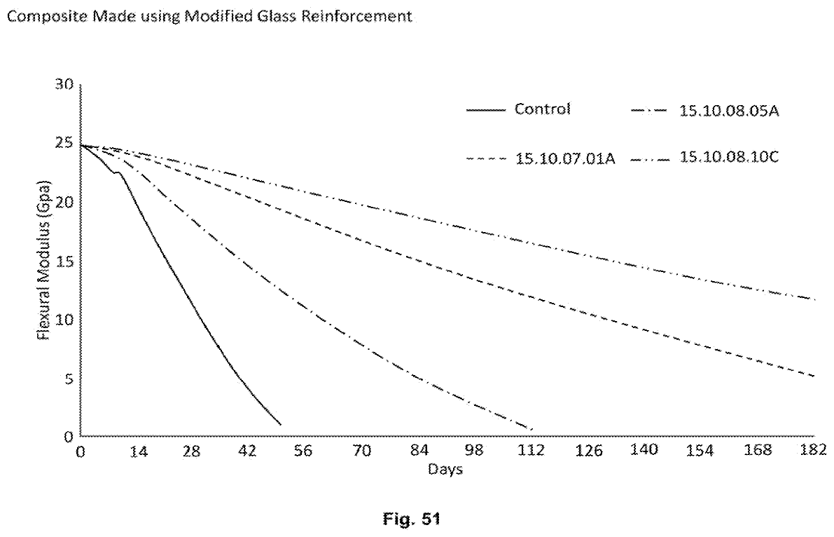

Example 72. PCL/PLA copolymer thermoplastic (Capa 8502A) is compounded with biodegradable glass (Mo-Sci Corp GL0122P/-53) and assessed for mechanical properties. Biodegradable glass is blended into thermoplastic at 5% glass volume and 25% glass volumes. Blends are molded into cubes (roughly 1 cm×1 cm×1 cm) and tested for compressive modulus. 5% glass volume cubes resulted in a 10% improvement in elastic modulus as compared to control cube of thermoplastic without glass. 25% glass volume cube resulted in a 68% improvement in elastic modulus as compared to control cube of thermoplastic without glass. The results are shown in FIG. 50.

Example 73. An FEA model is created to judge the requirements of an intra-medullar splint. The model is loaded with a 300N force at the proximal end of the bone (shoulder joint) and kept locked at the distal (elbow) end. The whole bone displacement at the proximal end of the bone is measured under unbroken, a partial proximal humeral fracture (a model of a fracture half-way through the bone) and while splinted with an intra-medullar splint with increasing step values of Young's modulus in the partial and full fracture bone. The results demonstrate that a splint with a Young's modulus of greater than 12 GPa is necessary to return the bone to its unbroken performance level.

Example 74. A bone break model is created with a composite tube (Garulite) with an 8.10 mm ID to empirically support the FEA model from Example 73. Nine 75 mm long flexible braided glass reinforcement rods as described in Examples 41-50 (between 30-40% FV) are loaded into a 10 mm diameter PET balloon through a tube that could only accept the rods one at a time. The bag and rods are positioned across an incomplete cut in the tube (approximately 0.7 mm in distance) and filled under vacuum from a single manually extended 60 cc syringe with the polyurethane from Example 51 then cured at 70° C. The tube break is tested pre and post splint positioning in non-destructive and destructive 4-point bend testing. In non-destructive testing, the load needed to cause strain at the fracture line of 0.5% increased from 28 N to 260 N. In destructive testing, the repair withstood 516 N prior to reaching 2% strain and yielded at about 3.5% strain at 800 N of loading with a peak load of 880 N and a non-catastrophic failure mode. Since bone typically breaks at 1.5-2% strain and will experience secondary bone healing between 2-10% strain, this example demonstrates that this invention, with a reasonable final fiber volume will increase the stiffness of a fractured tubular bone to a degree that it approaches the performance criteria of bone and will allow secondary healing to occur.

Example 75. Thermoplastic P4HB beads and PLA beads as received are mixed with phosphate based soluble glasses and incubated in phosphate-based buffer solution at 50° C. for 52 days in vials, 50/50 by weight. Buffer is changed periodically as pH shifted. Beads are dried thoroughly after 52 days and analyzed via GPC. For P4HB, the higher molecular weight portion (Mz) decreased significantly regardless of additive. Lower molecular weight portion (Mn) increased slightly more in control than in samples with additives. Addition of 1 glass type effected on speed of degradation for both high molecular weight portion (Mz) as well as lower molecular weight portion (Mn) of samples. For PLA, there is a large decrease in MW regardless of additive. Soluble glass 1 very slightly slow degradation while soluble glass 2 speeds it up. This example demonstrates that a thermoplastic, soluble glass composite degrades. Additionally, P4HB—known to degrade primarily by enzymatic degradation—is demonstrated to have increased hydrolytic degradation due to the addition of soluble glass.

Example 76. 2 mm pins are constructed as per Example 62 with the polyurethane of Example 51 and phosphate based soluble glass uni-axial fibers. The pins are coated with a well-established material that retards the ingress of water to a rate of 1 gram*mil/(100 in2)*day. The loss of stiffness is severely retarded over a 25-day period with a stiffness that remained well above the need expected in the FEA analysis from Example 73. This demonstrates that the use of an external barrier such as hydrophobic properties of the bag/balloon or an external coating on a pre-formed structure will serve to retard the degradation process of the full implant. See FIG. 51.

Example 77. The time it takes to fill a multi-braid structure is measured. A volumetric model is created with an increasing number of triangular braids (as per Examples 41-50) loaded horizontally. A polyurethane as per Example 51 (viscosity approximately 1000 cp) is filled under vacuum alone (no added positive mechanical pressure from the injection syringe) provided by a fully extended 60 cc syringe. The injection time is tracked along with the volume injected. The results are shown below (the fit lines are for visualization only, not a mathematical fit) for the highest fiber volume (# of braids) loaded per model size (described by model diameter). The models all had different overall volumes to fill but the same length (i.e., distance from bottom of model to top; the two largest models had approximately 61% fiber volumes to wet-out and the smallest model is a slightly higher fiber volume of 68% to fill. The fill and wet-out is completed in 90 seconds or less for the two largest volumes and took about 2 minutes for an “over-stuffed” small model. This demonstrates a reasonable fill time for in situ filling in an operative environment for building a splint. There are occasions when the reinforcing rods are too close to the inflow of the resin, this represents instances where the rod insertion could have kinked or blocked the inflow channels. These instances severely retarded inflow and reinforce the importance of relatively robust (but flexible) reinforcement rods. It also highlights the importance and addition of vacuum alone, from a simple disposable device (e.g., a syringe). See FIG. 55.

Example 78. Textile E glass braids are prepared having either 6 axial fiber bundles (predominantly circular cross-section) or 3 axial fiber bundles (predominantly circular cross-section) bound by bias fiber bundles orientated at +/−45° to the axial bundles in a glass content ratio of approximately 1:1 axial to bias fiber volume and designed to contain the same volume of fiber per unit length. 2 mm composite pins are built using the same polyurethane matrix and method of construction described in Examples 41-50. The flexural modulus of each are compared in FIG. 57. Thus, the shape of a single reinforcing element will not alter its ability to reinforce a matrix.

Example 79. Polyurethane matrix is combined with phosphate based soluble glass fibers to produce material pins as per Example 62. The pins are degraded in a buffer solution at 70° C. to accelerate degradation effects. The remaining weight compared to the starting level of soluble glass material is found to correlate with the degradation rate in a non-linear fashion as shown in FIG. 58, thus demonstrating control of degradation rate of the product.

Example 80. Thermoplastic PLA polymer is co-mingled with different types of phosphate based soluble glass fibers and submerged in a 7.4 pH buffer solution with periodic refreshes of the solution. FIG. 59 shows the resulting change in local environmental pH between PLA alone and PLA comingled with 2 different types of soluble glass fibers, each giving a different resulting environment.

Example 81. Thermoplastic Polyurethane polymer is co-mingled with different types of phosphate based soluble glass fibers and submerged in a 7.4 pH buffer solution with periodic refreshes of the solution. FIG. 60 shows the resulting change in local environmental pH between PLA alone and PLA comingled with 2 different types of soluble glass fibers, each giving a different resulting environment. Thus, demonstrating the ability to modify the local environment during or after material degradation.

Examples of Barriers Used in Biodegradable High Strength Composite Systems

Example 84. 2 mm diameter round pins (5 cm) long are made out of a biodegradable composite like example 62 in Ortho040506. One group of pins is coated with a non-degradable well established and measurable (WVP−1 g*mil/100 in2 day=0.4 g*mm/m2 day)) substance to a thickness of 0.5 mil (13 μm), a control group remained uncoated. The groups of pins are submerged in distilled water at room temperature (20° C.). The loss of mechanical properties (bending stiffness) is measured periodically. The rate of stiffness loss is reduced from 16% per day to 1.1% per day with the coating.

Example 85-89. A fully degradable pin with dimension from above can be reduced to practice using a number of available barrier substances described in literature as listed in the table below (all at temperatures between 20 and 25° C.):

| | | | Req | |
|---|---|---|---|---|
| | | WVP | Thick- | |
| Exam- | | (g mm/ | ness | |
| ple | Material | m²*day) | (μm) | Ref |
| 85 | 20 μm thick commercial Poly | 1.1 | 44 | [1] |
| | (Lactic Acid) Film | | | |
| 86 | Al₂O₃ Coated | 0.7 | 21 | [1] |
| | PLA Film | | | |
| 87 | Poly Hydroxybuterate-co-valerate | 0.3 | 11 | [2] |
| | (6% valerate) | | | |
| 88 | Poly (Lactic Acid) 66% | 2.1 | 66 | [2] |
| | Crystallinity | | | |
| 89 | Poly ε-capralactone | 4.4 | 143 | [2] |

This table shows that results similar to that above can be achieved with substances considered biodegradable or bioabsorbable with a single barrier layer between 0.8 and 11 times the thickness of the material in example 1 above.

Example 90-93. For the examples above, the temperatures studied are approximately 20-25° C. If the pin are to be designed for use inside of a body, as a small bone splint for example, then the temperature would be 37° C., resulting in more rapid diffusion of water (Brownian motion). A fully degradable pin with dimension from Example 1 above can still be reduced to practice with a variation of the thickness (calculated using an estimate of the Arrhenius equation):

| | | | Req | |
|---|---|---|---|---|
| | | WVP | Thick- | |
| Exam- | | (g mm/ | ness | |
| ple | Material | m²*day) | (μm) | Ref |
| 90 | 20 μm thick commercial Poly | 3.0 | 96 | calcu- |
| | (Lactic Acid) Film | | | lated |
| 91 | Poly Hydroxybuterate-co- | 0.8 | 25 | |
| | valerate (6% valerate) | | | |
| 92 | Poly (Lactic Acid) 66% | 4.9 | 159 | |
| | Crystallinity | | | |
| 93 | Poly ε-capralactone (PCL) | 10.6 | 343 | |

This table shows that results similar to Example 84 can be achieved with substances considered biodegradable or bioabsorbable with a single barrier layer between 2 and 27 times the thickness of the material in Example 84 above.

Example 94. The thicknesses of the coatings in Examples 90-93 are high for some of the more readily available and acceptable materials for human implantation. In addition, there may be a desire to exact a compliant device, such as an implantable medical balloon. Therefore, the thickness of the barrier would need to stay within the range of a compliant material. To exact this, an insoluble solid suspension is added to the polymer to reduce the permeability rate. It has been reported that amounts as small as 5 wt % of clay added to polymer can halve the permeability [3]. In the same publication, the effect on permeability rises exponentially for up to 20 wt % of additive. To reduce this to practice, the PCL from Example 93 above is compounded with 10 wt % biocompatible insoluble such as Mg(OH)2—which has a plate-like morphology after undergoing a specific heating profile. Mg(OH)2 is estimated to be half as effective as clays, therefore the effect is to reduce the WVP of the material from 10.6 to 5.3 g mm/m2*day. With this material the required barrier thickness is reduced from 343 to 171 μm (0.006″). Given the inherent flexibility of PCL, this is a good thickness for a compliant human implantable degradable balloon.

Example 95. An alternate example (to Example 109) of a barrier appropriate for use as a compliant human implantable degradable balloon is enacted by the use of multiple co-extruded layers of polymers. The calculated WVTR of the resulting films of examples 1-4 is 31 g/m2*day. To enact a thinner design with biologic benefits layers of a balloon can be created by co-extrusion of a tube and subsequent expansion using balloon forming methods. Other methods such as dip coating can also be used. The resulting WVTR is calculated using a parallel network equation.[4]

| | | WVP | Thick- | |
|---|---|---|---|---|
| | | (g mm/ | ness | WVTR |
| Layer | Material | m²*day) | (μm) | (g/m²*day) |
| Out | Poly (Lactic Acid) 66% | 4.9 | 25.4 | 193 |
| | Crystallinity/Hydroxy- | | | |
| | apatite Suspension | | | |
| Middle | Poly ε-capralactone | 5.3* | 25.4 | 209 |
| | (PCL)/Mg(OH)2 | | | |
| | 10 wt % suspension | | | |
| Inside | Poly (Lactic Acid) 66% | 2.5* | 50.8 | 48 |
| | Crystallinity/ | | | |
| | Mg(OH)2 10 wt % | | | |
| | suspension | | 100 | 33 |

*Estimated halved WVP due to insoluble components at 10 wt %

This example shows that by layering three dissimilar materials with different water transfer rates, a barrier with similar water permeability as those of examples 1 through 4 (33 g/m2*day vs. 31 g/m2*day) with a relatively thin material (100 μm, 0.004″). In addition, the material has an outer coating infused with Hydroxyapatite which is advantageous if implanted near bone, and the compliance and adhesive capability of the middle layer of PCL gives some resilience to the overall structure.

### REFERENCES FOR EXAMPLES 84-95

[1] T. Hirvikorpi, M. Vaha-Nissi, A. Harlin, M. Salomaki, S. Areva, J. T. Korhonen, and M. Karppinen, “Enhanced water vapor barrier properties for biopolymer films by polyelectrolyte multilayer and atomic layer deposited Al2O3 double-coating,” Appl. Surf. Sci., vol. 257, no. 22, pp. 9451-9454, September 2011.
[2] R. Shogren, “Water vapor permeability of biodegradable polymers,” J. Environ. Polym. Degrad., vol. 5, no. 2, pp. 91-95, 1997.
[3] J.-W. Rhim, H.-M. Park, and C.-S. Ha, “Bio-nanocomposites for food packaging applications,” Prog. Polym. Sci., vol. 38, no. 10-11, pp. 1629-1652, October 2013.
[4] K. Cooksey, “Interaction of food and packaging contents,” Intell. Act. Packag. Fruits Veg., pp. 201-237, 2007.

Example 96. A two-component polyurethane polymeric material is made by preparing (component A) polyol blend consisting of a polycaprolactone triol (70% by weight), 1,4;3,6-dianhydrous-d-sorbitol (15%), and a citric acid ester (15%). The polyol is mixed and crosslinked with a hexamethylene diisocyanate trimer (component B) to provide a polyurethane matrix that is used in the application to bind reinforcement fibers to ultimately form a composite. The isocyanate (NCO)/hydroxyl (OH) stoichiometry ratio is 1:1. Catalyst is added to polyisocyanate to catalyze the isocyananate-hydroxyl reaction which forms the polyurethane. Catalyst selection is based on compatibility and stability in the system. In these studies, a zirconium catalyst is added to the isocyanate prior to mixing the polyol and isocyanate components.

Viscosity. Viscosity for this application is important for the injection process of exiting the syringe, flowing through a static mixer (to mix components A and B), through the catheter, into the implant enclosure which contains reinforcing fibers. Proper viscosity is also critical such that the reinforcing fibers become completely “wet out” by polyurethane before gelling begins. centipoise, respectively. Similar viscosities of components A and B is critical to provide efficient and thorough mixing of the 2 components. The viscosity of the mixed components A and B one minute after mixing is 1440 cps.

Degradable polyols cooks

| diol/acid | mole ratio | *viscosity, cp |
|---|---|---|
| propylene glycol/caprolactone | 1.0/1.0 | 80 |
| propylene glycol/caprolactone | 1.0/2.0 | 190 |
| propylene glycol/caprolactone | 1.0/3.0 | 250 |
| isosorbide/caprolactone | 1.0/1.0 | 1520 |
| isosorbide/caprolactone | 1.0/2.0 | 1480 |
| isosorbide/caprolactone | 1.0/3.0 | solids |
| propylene glycol/L-lactide | 1.0/1.0 | 120 |
| propylene glycol/L-lactide | 1.0/2.0 | 580 |
| isosorbide/L-lactide | 1.0/1.0 | >6000 |
| CHDM/L-lactide | 1.0/1.0 | 3190 |
| propylene glycol/DL-lactide | 1.0/1.0 | 120 |
| propylene glycol/DL-lactide | 1.0/2.0 | 320 |
| isosorbide/DL-lactide | 1.0/1.0 | >6000 |
| CHDM/DL-lactide | 1.0/1.0 | 2950 |
| propylene glycol/glycolide | 1.0/1.0 | milky |
| propylene glycol/glycolide | 1.0/2.0 | 550 |
| isosorbide/glycolide | 1.0/1.0 | >6000 |
| CHDM/glycolide | 1.0/1.0 | 2140 |

*viscosity, cp: measured using Brookfield CAP 2000+ viscometer, at 25° C.

Exotherm. Proper curing of the polyol-isocyanate reaction to form the polyurethane described above causes an exotherm and results in development of critical mechanical properties. The reaction rate and the extent of exotherm can be controlled by altering amount of catalyst used, and by amount of reinforcement. Preparing a 5-gram sample of polyurethane with 0.17% zirconium catalyst resulted in a maximum temperature of 35° C. twenty-four minutes after mixing. Increasing the zirconium catalyst level to 0.3% resulted in a maximum temperature of 53° C. twelve minutes after mixing. The final matrix glass transition temperature is 46° C. with each of catalyst levels tested.

With 30% volume fiber reinforcement and 70% polyurethane matrix, and 0.17% zirconium catalyst, the maximum temperature of 24° C. occurs over a time frame of 10 to 20 minutes after mixing. This composite composition with 0.3% catalyst causes a maximum temperature of 33° C. twelve minutes after mixing.

Pot Life. As with exotherm, pot life can be controlled by varying amount of catalyst used in polyurethane. The polyurethane for this procedure has a usable working/application time (also known as potlife) in which it can be efficiently injected to wet out reinforcement fiber within the implant enclosure. An acceptable viscosity range has been observed to be roughly 500 cps-5000 cps. The useable working time (viscosity of mixed components A and B reaching 5000 cps) with 0.3% zirconium catalyst is six minutes. The working time of this same system with 0.2% zirconium catalyst is eleven minutes.

Mechanical Strength. Mechanical strength development is very important for implant performance, as it determines when the patient can be moved out of the operating room, and when the patient can support him or herself. A composition containing 50% (by volume) of the above polyurethane with 0.2% zirconium catalyst and 50% by volume glass fibers is prepared into specimens for flexural modulus testing. After 6 days curing at 37° C. the flexural modulus is 12 GPa.

Example 97. A polymer matrix consisting of both caprolactone and lactic acid groups is formulated. Component A of the polymer matrix consisted of a poly(caprolactone) triol (70% by weight), poly(caprolactone-co-lactide) triol (10% by weight), 1,4;3,6-dianhydrous-d-sorbitol (15%), and a citric acid ester (5%), plus a bismuth catalyst (0.09% by weight). The polyol (component A) is mixed and crosslinked with a hexamethylene diisocyanate trimer (component B) to provide a polyurethane matrix that is used in the application to bind reinforcement fibers so as to, ultimately, form a composite implant. The isocyanate (NCO)/hydroxyl (OH) stoichiometry ratio is 1.1. Viscosity of components A and B are 1210 and 1066 cPs, respectively. The pot life of the formulation is around 4 minutes. The maximum temperature reached is 65° C.; however, in the presence of 40% fiber volume, the maximum temperature reached is 45° C.

Example 98. Round pre-cured pins for intramedullary (IM) bone fixation with a 2 mm diameter and 3 cm long, are made out of a biodegradable composite, consisting of glass braid reinforcement, a polymer matrix, and an outer barrier. The glass braids are made out phosphate glass fibers with 3 axial fiber bundles (predominantly circular cross-section) bound by bias fiber bundles orientated at +/−45° to the axial bundles. Component A of the polymer matrix consists of a poly(caprolactone-lactic acid) triol (80% by weight), 1,4;3,6-dianhydrous-d-sorbitol (15%), and a citric acid ester (5%), plus a tin catalyst (0.13% by weight). The polyol is mixed and crosslinked with a hexamethylene diisocyanate trimer (component B), mixed with 15% hydroxyapatite, to provide a polyurethane matrix that is used in the application to bind reinforcement fibers to, ultimately, form a composite implant. The isocyanate (NCO)/hydroxyl (OH) stoichiometry ratio is 1.1. Hydroxyapatite is added to the polymer matrix as it is biocompatible, osteoinductive, and acts as degradation control buffer. The two parts (components A and B) are mixed in a ratio of 1:2 and reacted with the glass reinforcement (55% fiber volume) with the help of a tin catalyst at a concentration of 0.13% and cured at 70° C. overnight. The cured rods are coated with a 100-micron thick layer (i.e., a coating) that consists of an inner barrier layer and an outer compatibilizer layer. The inner barrier layer is a 75-micron thick water barrier layer and consists of high aspect ratio magnesium hydroxide microparticles (average size 10-micron diameter and 200 nm thick), dispersed in a degradable polyester polyurethane matrix. The outer compatibilizer layer consists of beta tricalcium phosphate, dispersed in a degradable polyester polyurethane matrix. To increase the adhesion of the coating to the pins, the pins are slightly roughened/structured. The coating has a water vapor permeability (WVP) of 2 g*mil/100 in2 day (0.8 g*mm/m2 day). Where the pins are used for hammer toe fixation, the cured pins are cut to the desired size, typically ranging from 2-3 cm. Similarly-formed pins, of appropriate size, can be used for other types of IM fixations, including small bones, clavicle, ribs, radius, ulna, etc.

This can also be seen in example 98 and 62 where nested bundles of reinforcing elements embedded in a matrix at approximately 71% fiber volume to create a pin or rod with a 2 mm diameter.

Example 100. This example is for pre-cured rods for fixation of tibia, femur, and humerus. Round rods with 12.5 mm diameter, and 30 cm long, are made out of a biodegradable composite, consisting of glass braid reinforcement, a polymer matrix, and an outer barrier. The glass braids are made from phosphate glass 6 axial fiber bundles (predominantly circular cross-section) bound by bias fiber bundles orientated at +/−45° to the axial bundles. The polymer matrix is a poly(caprolactone-co-lactic-co-glycolide) polyurethane system and consisted of a mixture of two parts. The first part (Part A) consists of a polyester polyol mixture with a catalyst, whereas the second part (Part B) consists of isocyanate prepolymer with 25% beta tricalciumphosphate, which is biocompatible, osseoinductive, and acts as buffer control. The isocyanate (NCO)/hydroxyl (OH) stoichiometry ratio is 1.05. The two parts are mixed in a ratio of 1:2 and reacted with the glass reinforcement (50% fiber volume) with the help of a tin catalyst at a concentration of 0.07% and cured at 70° C. overnight. The polyester groups in Part A impart degradability to the cured matrix, and consist of caprolactone, lactic acid, and glycolic acid groups. The cured rods are coated with a 50-micron thick vapor-deposited magnesium coating. To increase the adhesion of the barrier to the pins, the pins are slightly roughened/structured. This rod can then be used for tibia fixation (of femoral fixation, humeral fixation, etc.).

Example 101. This example discusses the splint system with a single pre-cured rod for intramedullary or other bone hole fixation. The diameter of the rod can be any diameter between 0.5 mm and 20 mm depending on the application. For some applications, the diameter of the rod can be between 1 mm and 7 mm. For some other applications, the diameter of the rod can be between 6.5 mm and 14 mm. The length of the rod can be between 0.5 cm and 46 cm depending on the application. For some applications, the length of the rod can be between 2 cm and 15 cm. For some other applications, the length of the rod can be between 12 cm and 30 cm. The rod is placed inside a deflated containment bag. The containment bag is then placed inside the intramedullary (IM) canal of the bone, or inside another bone hole, inflated to the dimensions of the IM canal (e.g., 12.5 mm diameter and 30 cm long) or inflated to the dimensions of another bone hole, and filled with two-part injectable matrix to occupy the remaining space. The pre-cured rod has the same composition as disclosed in Example 100 above, but without a barrier layer. The human implantable degradable containment bag is prepared by with multiple co-extruded layers of polymers, as discussed in Example 96 above. The calculated water vapor transfer rate (WVTR) of the resulting containment bag is 31 g/m2*day. The two-part injectable matrix consists of Part A and Part B. Part A consists of poly(caprolactone-lactic acid) triol (80% by weight), 1,4;3,6-dianhydrous-d-sorbitol (15%), and a citric acid ester (5%). Part B is hexamethylene diisocyanate trimer, mixed with 20% biphasic calcium phosphate. Part A and Part B are then injected through a catheter into the inflated containment bag and cured under physiological conditions. The resultant curing provides a solid composite implant that substantially conforms to the shape of the IM canal (or other bone hole). Such splint systems can be used for fixation of the tibia, clavicle, humerus, femur, radius, ulna, ribs, etc.

Example 102. This example discusses a splint system with multiple pre-cured pins (rods) for tibial fixation. Multiple pre-cured rods (10-12 in number) of diameter 2.2 mm and length of 28 mm are placed inside a deflated containment bag. The containment bag is then placed inside intramedullary (IM) canal of the tibia, inflated to the dimensions of the tibial IM canal (e.g., 12.5 mm diameter and 30 cm long), and filled with two-part injectable matrix to “glue” the pre-cured pins together and occupy the remaining space. The pre-cured pins consist of 65% phosphate glass reinforcement braids with remainder being a polymer matrix. The composition of the polymer matrix is similar to that disclosed in Example 97 above. The pot life of the two-part injectable matrix after mixing is about 3 minutes and reaches a maximum temperature of 45° C. during the cure. Such a composite implant requires only a smaller access hole (e.g., approximately 2.5-3 mm).

Depending on the pin configuration and IM canal diameter, multiple such pins can be fitted in the IM canal. The Table and figure below show some possible packing of cylindrical or triangular pins in a canal that is 8 mm in diameter.

| | Pin | Number of | Packing |
|---|---|---|---|
| Pin Configuration | Diameter | Pins | Fraction |
| 2 mm cylindrical pin | 2 | 11 | 0.69 |
| 2 and 1 mm cylindrical pin | 2 | 9 | 0.78 |
| | 1 | 14 | |
| 1 mm cylindrical pin | 1 | 51 | 0.80 |
| 2 mm triangular pin | 2 | 24 | 0.96 |

See FIG. 61.

Example 103. This example discusses a splint system with multiple pre-cured reinforcement braids (pins). Multiple reinforcement pins (10-12 in number) of diameter 2 mm and length of 28 mm are placed inside a deflated containment bag. The containment bag is then placed inside intramedullary (IM) canal of tibia (or other bone), inflated to the dimensions of the tibial IM canal (e.g., 12.5 mm diameter and 30 cm long), and filled with two-part injectable matrix to “glue” the pre-cured pins and occupy the remaining space. The composition of the polymer matrix is similar to that disclosed in Example 97 above. The pot life of the two-part injectable matrix after mixing is 3 minutes and reaches a maximum temperature of 45° C. during the cure. Such an implant requires a smaller access hole (e.g., approximately 2.25-3 mm).

Example 104. This example describes a process for forming sheets for coating pre-cured pins and rods. Polycaprolactone (MW of 50,000) is dissolved in ethyl acetate solvent at a concentration of 25%. Particles of beta tricalciumphosphate is then added to the solution at a concentration of 5%. The solution is then thoroughly mixed to dissolve the polycaprolactone (PCL) and efficiently disperse the particles. Sheets are then drawn out of the solution using a draw-down technique, and then dried in an oven at 40° C. to remove all the solvent.

Example 105. This example discusses a pre-cured rod (pin). The rod consists of four elements: glass fibers as reinforcement, sizing on glass fibers, matrix, and a coating. The glass fibers are biodegradable phosphate glass fibers that release sodium and calcium ions as the fibers degrade. A sizing of polyvinylalcohol is applied to the glass fibers for improved wettability of the polymer matrix. Component A of the polymer matrix consisted of a poly(caprolactone) triol (70% by weight), 1,4;3,6-dianhydrous-d-sorbitol (15%), and a citric acid ester (15%), plus a zirconium catalyst (0.3% by weight). The polyol (component A) is mixed and crosslinked with a hexamethylene diisocyanate trimer (component B) and 5% beta tricalciumphosphate, to provide a polyurethane matrix that is used in the application to bind reinforcement fibers so as to, ultimately, form a composite implant. The isocyanate (NCO)/hydroxyl (OH) stoichiometry ratio is 1.1. Hydroxyapatite is added to the polymer matrix as it is biocompatible, osteoinductive, and acts as degradation control buffer. The two parts are reacted with the glass reinforcement (30% fiber volume) with the help of a tin catalyst at a concentration of 0.07% and cured at 70° C. overnight. After the rods are cured, a coating is applied in two stages. In the first stage the rods are dip-coated in a solution of 18% polylactic acid and 2% magnesium hydroxide in ethyl acetate. The solvent is allowed to completely evaporate by placing them in a 70° C. oven for 1 hour. In the second stage, these rods are then coated with a sheet of polycaprolactone with beta tricalcium phosphate as disclosed in Example 104 above.

Example 106. In this example, a process for making degradable screws is described. A mold, having a cavity which is the shape of a screw, is made by drilling and tapping a Teflon block. Reinforcing glass braid is then inserted through the center of the screw-shaped cavity, followed by filling the cavity with a two-part curable polymer matrix as described in Example 97 above. The matrix is cured, followed by removal of the screw from the mold. An aluminum or stainless-steel mold can also be used for improved feature resolution. The matrix formulation can, optionally, contain 10% hydroxyapatite particles as an osteoinductive substance.

Example 107. The selection of a catalyst (see Examples 97-106 above) is dependent on multiple factors, for example, pot life, exotherm properties, mechanical properties, as well as potential foaming in case the injectable polymer matrix comes in contact with water.

| | | | | | | Foaming in Cured |
|---|---|---|---|---|---|---|
| | | | | Max Temp During Cure (Celsius) | | Matrix in |
| | | | | Pure Polymer | With 30% | Presence of |
| | | Pot Life | | Matrix | Fiber | Water |
| Tin | 0.05 | >30 | min | 38 | 27 | Excessive Foaming |
| | 0.09 | 12 | min | 57 | 31 | Excessive Foaming |
| | 0.13 | 3.5 | min | 68 | 48 | Excessive Foaming |
| | 0.2 | <1 | min | >85 | 77 | Some Foaming |
| Bismuth | 0.05 | >30 | min | 32 | 25 | Some Foaming |
| | 0.09 | 20 | min | 54 | 31 | Some Foaming |
| | 0.13 | 4 | min | 62 | 44 | Minimal/No Foaming |
| | 0.2 | <1 | min | >85 | 65 | Minimal/No Foaming |
| Zirconium | 10.05 | >30 | min | 21 | 21 | Does not Cure |
| | 0.09 | >30 | min | 21 | 21 | Does not Cure |
| | 0.13 | >30 | min | 25 | 22 | Some Foaming |
| | 0.2 | 25 | min | 31 | 25 | Some Foaming |
| | 0.3 | 6 | min | 43 | 35 | Some Foaming |

Example 108. Another important criteria is the hydroxyl content in the polyol part (the aforementioned component A) of the formulation. If the hydroxyl content is too low, the matrix may not cure, or may have lower mechanical properties. On the other hand, if the hydroxyl content is high, the implant can heat up significantly due to the heat generated during the crosslinking reaction. It is important for the hydroxyl content to be in the correct range to achieve the desired cure profile, exotherm properties, mechanical properties and Tg of the cured implant. For example, in a particular set of reactions for degradable polyester, the heat of reaction is 1.4 kcal/gram of hydroxyl groups. With hydroxyl content of 14% in the polyol component, there is a total heat release of 200 cal/g of the polyol component, which provides a maximum temperature of 44° C., and pot life of 4 minutes. However, with a hydroxyl content of 0.5% in the polyol component, we get a total heat release of 6.8 cal/g of the polyol component, which is generally not sufficient to cure the composite implant.

Example 110. As another example of layering materials for a desired goal, a biodegradable polymer, e.g., polycaprolactone (PCL), is wire coated over glass fibers to form a 2 mm diameter cord. The combination yields a useful cord with stiffness properties that can be used for a variety of applications such as parachute cords, fishing nets, etc. The glass fibers, when composed of a soluble glass and exposed to enough water, will change the micro-environment of the fiber and rapidly cause the degradation of the whole biocomposite leaving environmentally-neutral remains. This could be combined with a rapidly soluble glass and material that degrades by primarily enzymatic mechanisms such as with the poly-4-hydroxybutyrate (P4HB) above to create a cord that biodegrades in a very short time period.

Example 111. Specific to this example, PHAs like P4HB rely on enzymes for degradation. The ability to encourage nonenzymatic degradation will likely accelerate the combined rate of degradation and/or allow for degradation in environments free from biologic activity. Enzymatic degradation, a consumption process, is initiated from the surface inwards and therefore depends on a large surface area-to-mass ratio (such as films) in order to be classified as a biodegradable material according to ISO and ASTM standards. The application of the present invention allows simultaneous enzymatic degradation to occur in an outside-in manner, while the enzyme additive initiates and encourages degradation mechanisms within the center of the thicker materials. This will expand the range of materials that can be classified as biodegradable under ASTM standards, in addition the solubilized filler (glass fiber) will increase the porosity of the material to allow enzymatic degradation throughout the material by increasing the surface area of the material. As a further expansion of this concept, some of the enzyme additives can be produced in a fiber form and used, with appropriate compatibilizers, to increase the mechanical properties (e.g., strength and stiffness) of the material, thereby increasing the utility of the material to higher load-bearing applications while still maintaining a biodegradable classification. The high-performance polymers used for engineering applications are highly cross-linked to obtain the mechanical properties desired. These materials are not readily biodegradable in ambient conditions, if biodegradable at all in any relevant conditions. The [resent invention will allow biodegradable polymers to be used for higher performance applications. Currently, biopolymers such as those disclosed above (e.g., PH4B, PLA, etc.) are blended with polysaccharides to increase the degradation rate. The polysaccharides tend to be sticky and difficult to work with and have relatively poor mechanical properties. The mechanics of fiber-loaded composites are well known in the industry.

Example 112. Another example is the stiffening of a polymer with the addition of an additive (bioglass). In this case, a very low molecular weight polymer, one that can be heated to a low temperature and kneaded by hand with off-the-shelf bioglass (e.g., silicate-based soluble glass), is used. The bioglass additive is used to significantly increase the stiffness of the material, in addition, once dissolving, the bioglass will cause an internal shift to a basic environment, increasing the degradation rate. Given that the polymer is already at a low molecular weight, the time it takes to reach a 10,000 cp level for enzymes to complete the degradation process is much lower and makes this a very rapidly degrading material.

Example 113. Another example is a flexible composite (e.g., in the form of a rope, cord, sheet, mesh, tube, etc.) fabricated with the reinforcement fibers or braids along with a degradable polymer matrix. These flexible composites (e.g., in the form of a rope, cord, sheet, mesh, tube, etc.) have a bending modulus of preferably less than 5 GPa, preferably less than 3 GPa, and more preferably less than 1 GPa. The tensile modulus of the ropes, cords, sheets, meshes, tubes, etc. is preferably less than 200 GPa, preferably less than 150 GPa, and more preferably less than 100 GPa. Also, the tensile modulus of the flexible rope, cord, sheet, mesh, tube, etc. is at least 1 GPa, preferably at least 5 GPa, and more preferably at least 10 GPa.

Example 114. Another example of a containment device and dimensions is that of a small bone. The access to that bone could have dimensions of 0.25 mm. The containment bag wall thickness would be less than 0.125 mm. Containment bag expansion in the small bone could be 0.1 mm to 5 mm at the max diameter. The length of this containment bag could be 0.5 mm to 5 mm. This minimum volume of the containment bag is approximately 0.04 mm3 if the cavity is cylindrical. This would result in an expansion of 80%.

Example 115. Another example of a containment device and dimension is for a medium sized bone, such as a radius or ulna. The access to that bone could have dimensions of 3 mm. The containment bag wall thickness would be less than 0.15 mm. The containment bag would then expand to fill the cavity that could be as large as 30 mm at the maximum diameter. The length of the bone would range from 4 mm to 1250 mm which the containment bag could be designed to fit. The maximum volume of the containment bag is 883 cm3, 20,000%.

Example 116. Another example of a containment device and dimension is the delivery of a containment device into an access hole in the bone. The dimension of the access hole could be 40 mm. The containment bag can wall thickness could be as large as 20 mm with elongation. The total diameter of the containment bag would be larger than the access hole to create a fit into the cavity. The diameter of the containment bag for a large bone with an access hole of 40 mm, could be 15 cm at the largest diameter noting that the inflation of the containment bag would take the shape of the inner cavity. The length of the containment device could be long enough to fit in a bone for a giraffe which could be up to 25000 mm. The resulting volume of this containment device is 44,178 cm3.

Example 137: high strength and stiffness composite with enhanced toughness. This example discusses the composite with high strength, stiffness and enhanced toughness. Examples of various constructions of the composite are illustrated in FIGS. 31-36. The design of the composite produces a novel composite with strength and stiffness and enhanced toughness compared to a traditional composite made using similar components. The design enhances fracture toughness by at least 5%, more than 10%, more than 20% or more preferably more than 25%. Additionally, the composite design yields a composite material with a strain to yield of greater than 2%, at least 5%, at least 10%, or at least 20%.

### Example 138. Fibrous Bundles

Fibrous bundles, which may be a reinforcement element, are prepared having a multiple axially aligned fibers and a polymeric matrix, where each fiber includes a plurality of filaments. The fibers are arranged substantially in parallel with one another in the fiber bundle. A fibrous composite, or a composite element (either of which may be a reinforcement element) includes one or more discrete fibrous bundles. In the fibrous composite, the fibrous bundles may form discrete region in the composite with higher fiber volumes separated by matrix. The filament in a fiber may be twisted together clockwise (S), counterclockwise (Z) and not twisted (0). The fibers may also be characterized by the twist rate of the filaments. Examples of fibrous bundles are shown in Table A-1 below. The reinforcement elements of the fibrous bundles may be scaled. For example, two or more fibrous bundles may be nested together to form a larger structure of nested fibrous bundles, and the nested fibrous bundles may be assembled (e.g., by nesting) to form a larger structure, and so forth.

TABLE A-1
Reinforcement Element: Filament Bundles

| | Filaments | Average Fiber | Twist | Twist Rate |
|---|---|---|---|---|
| Sample | Quantity | Diameter (μm) | (S, Z, 0) | (per inch) |
| FB-1 | 10 | 9 | Z | 0.7 |
| FB-2 | 15 | 9 | Z | 0.7 |
| FB-3 | 20 | 9 | Z | 0.7 |
| FB-4 | 30 | 9 | Z | 0.7 |
| FB-5 | 40 | 9 | Z | 0.7 |
| FB-6 | 50 | 9 | Z | 0.7 |
| FB-7 | 100 | 9 | Z | 0.7 |
| FB-8 | 200 | 9 | Z | 0.7 |
| FB-9 | 400 | 9 | S | 0.7 |
| FB-10 | 400 | 9 | Z | 0.7 |
| FB-11 | 400 | 9 | 0 | 0 |
| FB-12 | 400 | 9 | Z | 3.5 |
| FB-13 | 600 | 9 | Z | 0.7 |
| FB-14 | 800 | 9 | Z | 0.7 |
| FB-15 | 1,000 | 9 | Z | 0.7 |
| FB-16 | 1,600 | 9 | Z | 0.7 |
| FB-17 | 2,000 | 9 | Z | 0.7 |
| FB-18 | 4,000 | 9 | Z | 0.7 |
| FB-19 | 10,000 | 9 | 0 | 0 |
| FB-20 | 15,000 | 9 | 0 | 0 |
| FB-21 | 20,000 | 9 | 0 | 0 |
| FB-22 | 25,000 | 9 | 0 | 0 |
| FB-23 | 30,000 | 9 | 0 | 0 |
| FB-24 | 40,000 | 9 | 0 | 0 |
| FB-25 | 50,000 | 9 | 0 | 0 |
| FB-26 | 75,000 | 9 | 0 | 0 |
| FB-27 | 100,000 | 9 | 0 | 0 |
| FB-28 | 125,000 | 9 | 0 | 0 |
| FB-29 | 150,000 | 9 | 0 | 0 |
| FB-30 | 300,000 | 9 | 0 | 0 |
| FB-31 | 630,000 | 9 | 0 | 0 |
| FB-32 | 1,000,000 | 9 | 0 | 0 |
| FB-33 | 190 | 13 | 0 | 0 |
| FB-34 | 145 | 15 | 0 | 0 |
| FB-35 | 80 | 20 | 0 | 0 |
| FB-36 | 50 | 25 | 0 | 0 |
| FB-37 | 35 | 30 | 0 | 0 |
| FB-38 | 25 | 35 | 0 | 0 |

Example 139 Examples of 0.5 mm 1 mm, 1.5 mm, 2 mm, (see table 5 mm 7.5 mm, 12 mm) (all axials=Brittle failure). Table A-2 illustrates various arrangements of nested fibrous bundles forming larger reinforcement elements. Fibrous bundles such as those illustrated in Table A-1, are nested to form the nested fibrous bundles (NFB) which are larger reinforcement elements. The nested fibrous bundles in samples NFB-1 to NFB-15 contain multiple fibrous bundles as shown in the Table A-2 below. The individual fibrous bundles have a shape that readily permits nesting. Some of the nested fibrous bundles are coated with matrix, which may form an outer region of a polymeric material around a core of the axially aligned fibers. Some of the nested fibrous bundles are used in the construction of larger structures. Mechanical testing and degradation testing are performed to characterize the properties of the reinforcement elements. The reinforcement elements in samples NFB-1 to NFB-7 contain nested bundles of fibrous bundles. The profile of the fibrous bundle, the fiber volume and quantity of fibrous bundles or are constant, but the number of filaments per fibrous bundle and the dis varied as shown in Table A-2. The filament bundles in the reinforcement elements contain filaments with different diameters, the quantity of filaments per bundle, and total filaments per reinforcement element. These factors are modified to maintain the fiber volume. The reinforcement elements in samples 8 to 9 contained nested bundles of reinforcement elements. The profile of the reinforcement elements, filament diameter, twist, twist rate, and total filaments per reinforcement element remained constant. The filament bundles in the reinforcement elements contain different filaments with different diameters, quantity of filaments per bundle, fiber volume.

Nested reinforcement elements (2 mm “rods”). Changed the filament diameter. The quantity of filaments per bundle is adjusted to maintain the fiber volume and cross sectional are of the reinforcement elements.

In the reinforcement elements in samples 7 and 8 the filaments per bundle are changed. The quantity of filament bundles per reinforcement element are adjusted to maintain the cross-sectional area of the reinforcement element and the fiber volume. In the reinforcement elements in samples 11 and 12 the twist, twist rate of the filament bundles are changed. The quantity of filament bundles is adjusted to maintain the fiber volume and cross-sectional area of the nested reinforcement element. In the reinforcement elements in samples 13, 14, 15 the qty of bundles and the area of the cross section of the reinforcement elements are changed.

Nested reinforcement elements (2 mm rods). Fixed variables: bundles single dimension (cell), fiber volume, and quantity of filaments in bundle. Variables: fiber diameter in bundles and quantity of filaments per bundle.

TABLE A-2
Nested Fibrous Bundles

| | | | Number of | | | |
|---|---|---|---|---|---|---|
| | Filament | Filaments | fibrous | | Twist | Total |
| | Diameter | per bundle | bundles | | (S, | Filaments |
| Sample | (μm) | (n_{f}) | (n_{b}) | Twist | Z, 0) | (n_{f} × n_{b}) |
| NFB-1 | 9 | 400 | 60 | 0.7 | Z | 24,000 |
| NFB-2 | 13 | 190 | 60 | 0.7 | Z | 11,400 |
| NFB-3 | 15 | 145 | 60 | 0.7 | Z | 8,700 |
| NFB-4 | 20 | 80 | 60 | 0.7 | Z | 4,800 |
| NFB-5 | 25 | 50 | 60 | 0.7 | Z | 3,000 |
| NFB-6 | 30 | 35 | 60 | 0.7 | Z | 2,100 |
| NFB-7 | 35 | 25 | 60 | 0.7 | Z | 1,500 |
| NFB-8 | 9 | 800 | 30 | 0.7 | Z | 24,000 |
| NFB-9 | 9 | 200 | 120 | 0.7 | Z | 24,000 |
| NFB-10 | 9 | 400 | 60 | 0.7 | S | 24,000 |
| NFB-11 | 9 | 400 | 63 | 0 | 0 | 25,200 |
| NFB-12 | 9 | 400 | 55 | 3.5 | Z | 22,000 |
| NFB-13 | 9 | 400 | 4 | 0.7 | Z | 1,600 |
| NFB-14 | 9 | 400 | 16 | 0.7 | Z | 6,400 |
| NFB-15 | 9 | 400 | 35 | 0.7 | Z | 14,000 |

* Average value

Binding Reinforcement Elements/Bias Fiber Elements

The bias fiber elements are prepared to be flexible so that they can be processed, wound, and woven into a braided structure or other bias structure. It may also be important for the bias fiber element to have a structure that permits flat contact between elements braided in a positive and negative angle. The bias element may have a tape like structure, with an aspect ratio of 2 or more, 3 or more, or 4 or more.

Some of the variables which are explored in the textiles and composites include the number of fibrous bundles, the ratio of the inner core to the outer region, nesting of the fibrous bundles, the shape of the fibrous bundles, the amount and type of bias fiber elements, and the volume ratio of the (A, axial) fibers in the fibrous bundles to the amount of (B, bias) fibers in the bias reinforcement elements. Composite structures in the form of pins having a diameter or thickness of about 0.5 mm, about 1 mm, about 1.5 mm, or about 2 mm are prepared. The nested fibrous bundles are bound by together by the bias fiber elements. The binding of the fibers is using one or more braids around the core.

TABLE A-3

| | Sample | Sample | Sample |
|---|---|---|---|
| | B-1 | B-2 | B-3 |
| Nested and Bound | | | |
| Reinforcement Element | | | |
| Shape | C, H, S, E. | C, H, S, E. | C, H, S, E. |
| | O, T, P | O, T, P | O, T, P |
| A:B Ratio | 1:1 | 1:1 | 1:1 |
| Weight per foot (grams/foot) | 1.3 | 1.3 | 1.3 |
| Fiber Volume (%) (target | 52 | 50 | 50 |
| 50% to 65%) | | | |
| FV tolerance (%) | 5 | 5 | 5 |
| Fiber Diameter (μm) | 9 | 9 | 9 |
| Total Filaments | 21600 | 21600 | 21600 |
| Inner Core Reinforcement | | | |
| Elements | | | |
| Total Axial Nested RE | 30 | 60 | 120 |
| Filaments per Axial RE | 400 | 200 | 100 |
| Bundle | | | |
| Total Axial RE fibers | 12000 | 12000 | 12000 |
| Shape | C, H, S, E. | C, H, S, E. | C, H, S, E. |
| | O, T, P | O, T, P | O, T, P |
| Unit Cell (bundle size) | | | |
| Outer Core | | | |
| Bias Angle (+−degrees) | 45 | 45 | 45 |
| Tolerance Degrees | 3 | 3 | 3 |
| Total Transverse RE | 24 | 48 | 96 |
| bundles | | | |
| Fibers Per Transverse RE | 400 | 200 | 100 |
| Fibers per Transverse RE | 800 | 800 | 800 |
| bundle | | | |
| Total Transverse RE fibers | 9600 | 9600 | 9600 |
| Shape | E | E | E |

*C = Circle; O = ovular; E = elliptical; T = triangular; P = polygon; S =; H = hexagonal

TABLE 4a

| | Sample | Sample | Sample | Sample |
|---|---|---|---|---|
| | B-4 | B-5 | B-6 | B-7 |
| Nested and Bound | | | | |
| Reinforcement Element | | | | |
| Shape | C, H, S, E. | C, H, S, E. | C, H, S, E. | C, H, S, E. |
| | O, T, P | O, T, P | O, T, P | O, T, P |
| A:B Ratio | 1:1 | 1:1 | 1:1 | 1:1 |
| Weight per foot (grams/foot) | 1.3 | 1.3 | 1.3 | 1.3 |
| Fiber Volume (%) (target | 50 | 50 | 50 | 50 |
| 50% to 65%) | | | | |
| FV tolerance (%) | 5 | 5 | 5 | 5 |
| Fiber Diameter (μm) | 9 | 9 | 9 | 9 |
| Total Filaments | 21600 | 21600 | 24000 | 16800 |
| Inner Core Reinforcement | | | | |
| Elements | | | | |
| Total Axial Nested RE | 12 | 6 | 36 | 18 |
| Filaments per Axial RE | 1000 | 2000 | 400 | 400 |
| Bundle | | | | |
| Total Axial RE | 12000 | 12000 | 14400 | 7200 |
| fibers | | | | |
| Shape | C, H, S, E. | C, H, S, E. | C, H, S, E. | C, H, S, E. |
| | O, T, P | O, T, P | O, T, P | O, T, P |
| Unit Cell (bundle size) | | | | |
| Outer Core | | | | |
| Bias Angle (+−degrees) | 45 | 45 | 30 | 55 |
| Tolerance Degrees | 3 | 3 | 3 | 3 |
| Total Transverse RE | 24 | 24 | 24 | 24 |
| bundles | | | | |
| Fibers Per Transverse RE | 400 | 400 | 400 | 400 |
| Fibers per Transverse RE | 800 | 800 | 800 | 800 |
| bundle | | | | |
| Total Transverse RE fibers | 9600 | 9600 | 9600 | 9600 |
| Shape | E | E | E | E |

*C = Circle; O = ovular; E = elliptical; T = triangular; P = polygon

Interlocked Reinforcement Elements

In addition to/or instead of braiding around a core having multiple fibrous bundles, it is possible to interlock the fibrous bundles using the bias fiber elements. The fibrous bundles can be interlocked individually or in groups (such as groups of nested fibrous bundles). 1 mm, 1.5 mm, and 2 mm braided structures are prepared.

Table 4A illustrates

TABLE A-4

| | 3 axials | | | | 4 axials |
|---|---|---|---|---|---|
| | Sample | Sample | Sample | Sample | Sample |
| | 1 | 2 | 3 | 4 | 5 |
| Nested and Interlocked | | | | | |
| Reinforcement Element | | | | | |
| Reinforcement Element | | | | | |
| Shape | T | T | T | T | S, CR |
| A:B Ratio | | | | | |
| Weight per foot (grams/foot) | 1.3 | 1.3 | 1.3 | 1.3 | 1.3 |
| Fiber Volume (%) (target | 52 | 50 | 55 | 50 | 50 |
| 50% to 70%) | | | | | |
| Fiber Diameter (μm) | 9 | 9 | | 9 | 9 |
| Total Filaments | 21600 | 24000 | | 16800 | 21600 |
| Axial Reinforcement Elements | | | | | |
| Nested RE bundles per | 10 | 12 | 6 | 20 | 15 |
| Axial bundle | | | | | |
| QTY of Axial Bundles | 3 | 3 | 3 | 3 | 4 |
| Total Nested RE bundles | 30 | 36 | 18 | 60 | 60 |
| Fibers per RE bundle | 400 | 400 | 400 | 200 | 200 |
| Fibers per Axial RE bundle | 4000 | 4800 | 2400 | 4000 | 3000 |
| Total Axial filaments | 12000 | 14400 | 7200 | 12000 | 12000 |
| Volume Axial Filaments (%) | 50 | | | | |
| Shape | C, E | C, E | C, E | C, E | C, E |
| Transverse Reinforcement | | | | | |
| Elements | | | | | |
| Bias Angle (+−degrees) | 45 | 30 | 55 | 45 | 45 |
| Tolerance Degrees (+−) | 3 | 3 | 3 | 3 | 3 |
| Total Transverse RE bundles | 24 | 24 | 24 | 24 | 48 |
| Fibers Per Transverse RE | 400 | 400 | 400 | 200 | 200 |
| Total Transverse RE fibers | 9600 | 9600 | 9600 | 4800 | 9600 |
| Shape | E, S, O, | E, S, O, | E, S, O, | E, S, O, | E, S, O, |

| | | | | | | | | 8 |
|---|---|---|---|---|---|---|---|---|
| | 6 AXIALS | | | | | | | AXIALS |
| | Sample | Sample | Sample | Sample | Sample | Sample | Sample | Sample |
| | 6 | 7 | 8 | 9 | 10 | 11 | 12 | 13 |
| RE Nested and Interlocked | | | | | | | | |
| Reinforcement Element | | | | | | | | |
| Shape | H | H | H | H | H | H | H | 0 |
| A:B Ratio | | | | | | | | |
| Weight per foot (grams/foot) | 1.3 | 1.3 | 1.3 | 1.3 | 1.3 | 1.3 | 1.3 | 1.3 |
| Fiber Volume (%) (target | 52 | 50 | 50 | 50 | 50 | 50 | 50 | 50 |
| 50% to 70%) | | | | | | | | |
| Fiber Diameter (μm) | 9 | 9 | 9 | 9 | 9 | 9 | 9 | 9 |
| Total Filaments | 21600 | 21600 | 21600 | 21600 | 21600 | 24000 | 16800 | 21600 |
| Axial Reinforcement Elements | | | | | | | | |
| Nested RE bundles per | 5 | 10 | 20 | 2 | 1 | 6 | 3 | 15 |
| Axial bundle | | | | | | | | |
| QTY of Axial Bundles | 6 | 6 | 6 | 6 | 6 | 6 | 6 | 8 |
| Total Nested RE bundles | 30 | 60 | 120 | 12 | 6 | 36 | 18 | 120 |
| Fibers per RE bundle | 400 | 200 | 100 | 1000 | 2000 | 400 | 400 | 100 |
| Fibers per Axial RE bundle | 2000 | 2000 | 2000 | 2000 | 2000 | 2400 | 1200 | 1500 |
| Total Axial filaments | 12000 | 12000 | 12000 | 12000 | 12000 | 14400 | 7200 | 12000 |
| Volume Axial Filaments (%) | | | | | | | | |
| Shape | C, E | C, E | C, E | C, E | C, E | C, E | C, E | |
| Transverse Reinforcement | | | | | | | | |
| Elements | | | | | | | | |
| Bias Angle (+−degrees) | 45 | 45 | 45 | 45 | 45 | 30 | 55 | 45 |
| Tolerance Degrees (+−) | 3 | 3 | 3 | 3 | 3 | 3 | 3 | 3 |
| Total Transverse RE bundles | 24 | 48 | 96 | 24 | 24 | 24 | 24 | 48 |
| Fibers Per Transverse RE | 400 | 200 | 100 | 400 | 400 | 400 | 400 | 200 |
| Total Transverse RE fibers | 9600 | 9600 | 9600 | 9600 | 9600 | 9600 | 9600 | 9600 |
| Shape | E, S, O, | E, S, O, | E, S, O, | E, S, O, | E, S, O, | E, S, O, | E, S, O, | E, S, O, |

The composites can be scaled to larger structures. For example, multiple of the above smaller rods can be combined to form larger composites, such as larger rods having a diameter or thickness of 5 mm or more, about 7.5 mm or more, or about 12 mm or more. In preparing the larger rods, the smaller composite elements may be combined with matrix material, additional bias fiber elements (such as by binding and/or interlocking).

### Current Orthopedic Composites

A composite implant (bioabsorbable screw) produced and tested by Felfel and the University of Nottingham¹ comprises stacked laminates of polylactic acid (PLA) and continuous phosphate-based glass (PBG) fibers. Unidirectional fiber mats were produced from continuous 180 mm fiber bundles aligned and sized with a hydroxyethyl cellulose solution, washed, and dried. PLA films were prepared by compression molding pellets of PLA resin into approximately 0.2 mm thick films. The films and mats were then alternately stacked in a mold cavity (160 mm×160 mm×3 mm). The stack was then heated in a press ¹Felfel R (2013) Manufacture and characterization of bioabsorbable fibre reinforced composite rods and screws for bone fracture fixation applications, in mechanical engineering. University of Nottingham, Nottingham for 15 minutes at 210° C., pressed for 15 minutes at 38 bar, and then transferred to a cooling press and subjected to 38 bar for an additional 15 minutes. The sheets formed from these laminates were then cut into 15 mm×40 mm samples. Composite bars were fabricated from these samples and cut into 6.5 mm×4.5 mm×40 mm bars. These bars were loaded into a custom-made screw mold and placed in a heated press at 90° C. for 10 minutes. The mold was then pressed for 30 seconds at 3 bar. The side of the bar was compressed via a threaded rod to compress the bar into the head shape for a screw. The mold was then transferred to a cooling press at 3 bar for 5 minutes. Finished screws had dimensions of 6 mm major diameter, 4.75 mm minor diameter, and 32 mm length. Fiber volume of the finished screws were 31±2%. The most frequent and difficult clinical problem with screws is excessive torsional shear resulting in screw heads separating from the screw shaft, leaving behind a partial screw that is unable to perform its desired function and is difficult and time consuming to remove. The torsional stiffness of the screws produced were increased relative to unreinforced screws as anticipated. However, the fiber reinforced stacked laminate screws delaminated and suffered complete separation of the screw shaft when placed under torsional loads and broke at smaller angles of rotation and lower loads than unreinforced screws.

A commercially available bioabsorbable implant Corbion's (formally Vioxid) FiberLive®, described in WO 2010/122098 A2. The implant comprises bioglass and 70/30 poly-1 d-lactide (PLDLA). Combining these materials with commercially available fiber reinforced thermoplastic tapes technology, similar to the laminate taught above, to make glass reinforced bioabsorbable tapes for making laminate composite using established processes. Fiber reinforced tapes typically have fiber with a diameter from 3 μm to 35 μm with 10 to 1000 fibers or more per tape bundle. The thickness of the tapes typically ranges from about 0.2 mm to 1000 mm, and the width to thickness ratios are from 1:1 to 200:1 The profile of tape from can be circular, ovular or elliptical. Using thermoplastic reinforced tapes provides a wide array of reinforcement options and uses established manufacturing processes. Selection of the tape width is determined by the dimensions of the part being produced and the amount of glass needed to provide the desired strength. Obviously tapes for producing a bioabsorbable implant would be on the smaller end of the width to thickness range given their size, most likely in the 1:1 to 10:1 range. Wrapping the tape in layers and alternating the angle of the wrap to the longitudinal axis of the implant will provide a laminate construction that would have improved torque load performance by aligning fibers in the direction of loading. For example, alternating layers at +45°, −45°, and 0° to the longitudinal axis would produce a composite that had improved performance. Combining these bias tape wrapped layers with longitudinal fiber bundle layers can provide a composite that yields sufficient flexural stiffness and strength, compressive stiffness and strength, and torsional stiffness and strength. The Rule of Mixtures is used to predict the expected mechanical and physical properties the composite. Using the established degradable materials and manufacturing processes would have improved performance when compared to Felfel's construction. However, this composite construction will still suffer from the design trade-offs associated with typical composites. The trade-off for the laminated composite design and having a higher fiber volume to improve strength and stiffness is lower strain at yield, lower strain at failure, delamination and catastrophic failure. Clearly there is a need for composite designed that overcomes these shortcomings.

It should be understood that many additional changes in the details, materials, steps and arrangements of parts, which have been herein described and illustrated in order to explain the nature of the present invention, may be made by those skilled in the art while still remaining within the principles and scope of the invention.

As used herein, unless otherwise stated, the teachings envision that any member of a genus (list) may be excluded from the genus; and/or any member of a Markush grouping may be excluded from the grouping.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time and the like is, for example, from 1 to 90, preferably from 20 to 80, more preferably from 30 to 70, it is intended that intermediate range values such as (for example, 15 to 85, 22 to 68, 43 to 51, 30 to 32 etc.) are within the teachings of this specification. Likewise, individual intermediate values are also within the present teachings. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner. As can be seen, the comparative teaching of amounts expressed as weight/volume percent for two or more ingredients also encompasses relative weight proportions of the two or more ingredients to each other, even if not expressly stated. For example, if a teaching recites 2% A, and 5% B, then the teaching also encompasses a weight ratio of A:B of 2:5. Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes.

The term “consisting essentially of to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of (namely, the presence of any additional elements, ingredients, components or steps, does not materially affect the properties and/or benefits derived from the teachings; or even consist of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps. All references herein to elements or metals belonging to a certain Group refer to the Periodic Table of the Elements published and copyrighted by CRC Press, Inc., 1989. Any reference to the Group or Groups shall be to the Group or Groups as reflected in this Periodic Table of the Elements using the IUPAC system for numbering groups.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

### REFERENCE NUMERALS

5 Composite implant
6 Facet
7 Edge
8 Lobe
9 Longitudinal axis
10 Containment bag
11 Cannulation
12 Drive socket
13 Minor axis
14 Major axis
15 Reinforcement element
17 Outer region
20 Polymeric material
21 Core
22 Sheet
23 Coating
27 Axial reinforcement element
28 Bias reinforcement element
29 Tube
30 Roll
31 Tapered end
35 Rod
38 Crack path
40 Filaments
46 Resilient element
50 Access hole
51 Bone
52 Access port
53 Fracture
55 Catheter
56 Intramedullary canal
60 Rotatable flexible rod
75 Guidewire
80 Fibrous bundle
81 Fiber
82 Fibrous composite

### Definitions

Multi-phase material. Exhibits a substantial proportion of the properties of two phases.

Prepreg tape. A thin sheet consisting of fibers pre-coated with a polymer (thermoplastic or thermoset) matrix. Composites are frequently manufactured using prepreg tape. Laminate composites are typically fabricated by “laying-up” the layers of reinforcement sheets at the required orientation. Then elevated temperature and pressure are used to bond the assembly together.

Filament winding and tape winding or tape wrapping. Filament winding and tape winding or wrapping are methods used to construct laminate constructions of round or tubular shape. The process involves placing a mandrel or core on a rotating axis. A continuous length of fiber strand (individual filament, bundles, tows, yarns, roving, or tape) is then wound over the rotating core or mandrel. Fiber reinforced tapes typically have fibers with a diameter from 3 μm to 35 μm with 10 to 1000 fibers or more per tape bundle. They can have up to hundreds of thousands depending on the size of the part to be made. Commercial fiber reinforced tapes can be purchased in thicknesses from 0.20 mm to 1 mm. This will provide composites with thin layers from 0.20 mm to 1 mm in thickness. The tapes are fabricated by making films up to 3 meters in width. These rolls of film can then be slit to any narrower width tapes to be used as appropriate to the size of the part being fabricated. The width to thickness ratio of the tapes is typically from 1:1 to 200:1. The cross-sectional profile of tape can be circular, ovular or elliptical. These tapes are typically 50-80% fiber by weight or roughly 30-65% by volume, depending on the polymer and type of fiber used. The angle of the winding can be adjusted over a wide range of almost 0° to almost 90° to the axis of rotation.

Stacking sequence. The following are typical examples of stacking sequences for laminate composites. Orienting layers 1, 2, 3, 4, 5, 6, 7, and 8 at angles of 0°, +45°, −45°, 90°, 90°, −45°, +45°, and 0°, respectively. Orienting layers 1, 2, 3, 4, 5, and 6 at angles of +45°, 0°, −45°, +45°, 0°, and −45°, respectively. The orientation of the layers and reinforcement fibers are designed to provide the appropriate properties in multiple directions, such as strength and stiffness in bending, compression, or torsion. Such that the material may have isotropic (i.e., an object having a physical property which has the same value when measured in different directions) or anisotropic (i.e., an object having a physical property that has a different value when measured in different directions) properties.

The Rule of Mixtures. The Rule of Mixtures approximates material properties exhibited by an object based upon its material makeup and construction so that various design changes may be approximated. For example, a laminate could be produced by using silicate glass (2.58 g/cm³ density; 4200 MPa tensile strength; 72.5 GPa tensile modulus) reinforced polyester (1.27 g/cm³ density; 39.8 MPa tensile strength; 2.83 GPa tensile modulus) tape of 0.21 mm thickness and 50% fiber volume. The tape may be wrapped into the shape of a 2.5 mm diameter pin with varying fiber orientation (alternating between ±45° and 0°), with respect to the longitudinal axis of the pin, in alternating layers. The fibers oriented along the longitudinal axis (0°) may provide full properties proportional to their volume loading. The fibers oriented at ±45° may not contribute fully to the physical properties in tension, and therefore will need to have an efficiency factor applied. The physical properties of the composite pin can therefore be determined using the rule of mixtures. The density of the pin may be 1.93 g/cm³ ((0.5*1.27)+(0.5*2.58)). The tensile strength of the pin may be 839 MPa ([0.66*Cos 4)(45° *(0.5*4200)]+[0.33*Cos 4)(0° *(0.5*4200)]+(0.5*39.8)). The tensile modulus of the pin may be 21 GPa ([0.66*Cos 4)(45° (0.5*72.5)]+[0.33*Cos 4) (0° (0.5*72.5)]+(0.5*2.83)).

Fracture toughness. Fracture toughness is the resistance of materials to the propagation of flaws under an applied stress. It is generally assumed that the longer the flaw relative to the thickness of the part, the lower the stress is required to cause fracture. High fracture toughness in metals is generally achieved by increasing the ductility at the expense of lower yield strength. Fiber reinforced composites are generally anisotropic with the highest fracture resistance occurring with breakage and pull-out of the fibers and lowest resistance occurring in interlaminar planes.

Brittle failure. Brittle failure (failure mode) refers to the breakage of a material due to a sudden fracture. When a brittle failure occurs, the material breaks suddenly instead of deforming or straining under load.

Ductile failure. A ductile failure is a type of failure seen in malleable materials characterized by extensive plastic deformation or necking. This usually occurs prior to the actual failure of the material.

Strain to yield. The yield point is the point on a stress-strain curve that indicates the limit of elastic behavior and the beginning of plastic behavior. Strain is a measure of deformation of a material. The strain may be applied by bending, torsion, or compression. The bending may be tested by 3-point bend testing, 4-point bend testing, gap testing, or any combination thereof. The compression may be tested by standard testing, gap testing, or both.

Strain to failure. The failure may refer to brittle failure and/or ductile failure. Strain is a measure of deformation of a material. The strain may be applied by bending, torsion, or compression. The bending may be tested by 3-point bend testing, 4-point bend testing, gap testing, or any combination thereof. The compression may be tested by standard testing, gap testing, or both.

Elastic modulus. The elastic modulus is a measure of an object's resistance to being deformed elastically (i.e., non-permanently) when a stress is applied. The elastic modulus may be measured in bending, torsion, or compression. The bending may be tested by 3-point bend testing, 4-point bend testing, gap testing, or any combination thereof. The compression may be tested by standard testing, gap testing, or both.

Compression. Mechanical properties of a composite material in compression may be measured according to ASTM D3410/D3410M-16. The mechanical properties may be measured with the axial direction of the axial fibers (e.g., axial fiber bundles) arranged perpendicular to the direction of compression or parallel to the direction of compression. Unless otherwise specified, the test is conducted with the axial fiber arranged perpendicular to the direction of compression.

Torsion. Mechanical properties of a composite material in torsion may be measured according to ASTM D1043-16. Unless otherwise specified, the torsional properties are measured with the axial direction of the axial fibers generally aligned parallel to the direction of the length of the test specimen.

Flexural Properties. Flexural properties of a composite material may be measured according to ASTM D790-17. Unless otherwise specified, the flexural properties are measured with the axial direction of the axial fibers (e.g., axial fiber bundles) parallel to the direction of the length of the test specimen.

Tensile. Tensile properties of a composite material may be measured according to ASTM D638-14. Unless otherwise specified, the mechanical properties are measured with the axial direction of the axial fibers (e.g., axial fiber bundles) parallel to the direction of the length of the test specimen.

Glass transition temperature (Tg) of the reactant can be obtained by measurements or also by calculation using the William Landel Ferry Equation (WLF) M. L. Williams, R. F. Landel and J. D. Ferry, J. Am. Chem. Soc. 77,3701(1955). The website http://www.wernerblank.com/equat/ViSCTEMP3.htm provides a simple method to convert viscosity of an oligomeric polymer to the Tg.

The linear-elastic fracture toughness of a material is determined from the stress intensity factor (K) at which a thin crack in the material begins to grow. (https://en.wikipedia.org/wiki/Fracture_toughness).

Specific gravity is measured according to ASTM D792.

Fiber volume may be measured according to ASTM D3171-15

The melt flow rate is measured according to ASTM D1238. Unless otherwise specified, the melt flow rate is measured at 180° C./2.16 kg.

The viscosity is measured according to ASTM D445-19a.

1. A coated fiber adapted for use in a composite with a polymeric matrix comprising a plurality of aligned inorganic filaments, the coated fiber having a substantially uniform profile along its length, wherein the filaments have a polymeric or polymerizable coating over at least a portion of their surface, wherein the structure of the fiber and the materials of the filament and the coating are selected for providing the following characteristics:
i) the coated fiber has a tensile modulus of about 10 GPa or more, as measured according to ASTM D638-14, using a fiber shaped specimen;
ii) the coated fiber has a flexural modulus of about 5 GPa or more, as measured according to ASTM D 790-17; and
the coated fiber has a bending radius of about 10 cm or less, as measured according to ASTM E290-14 (semi-guided bend test around a mandrel, preferably where the coated fiber is bent about 180°);
wherein the filaments are twisted.

2. The coated fiber of claim 1, wherein
the filaments are twisted at a twist rate of about 0.10 turns per cm or more to about 10 turns per cm or less; and
the coated fiber has a cross-section perpendicular to the length direction characterized by an aspect ratio of about 1.2 or more.

3. The coated fiber of claim 1
wherein the filaments have a diameter of about 1 μm to about 50 μm; and
wherein a concentration of the inorganic filaments in the coated fiber is about 55 volume percent or more;
optionally wherein the coated fiber has a cross-section perpendicular to the length that is generally circular, generally oval shaped, generally elliptical, or generally polygonal.

4. (canceled)

5. (canceled)

6. The coated fiber of claim 1, wherein the inorganic filaments are formed of a glass;
optionally wherein the glass and/or the polymeric material are biodegradable or bioresorbable.

7. The coated fiber of claim 6, wherein the glass includes a first alkaline or alkaline earth metal, wherein a concentration of the first alkaline earth or alkaline earth metal in the glass at a region near the surface of the filament is different from a concentration in the glass near the center of the filament;
optionally, wherein a difference in the composition of the glass provides a surface region that is more resistant to dissolution in water compared to a core region;
optionally, wherein the coated fibers are impregnated with the polymeric material.

8. (canceled)

9. (canceled)

10. (canceled)

11. The coated fibers of claim 1, wherein the coated fiber is characterized by one or any combination of the following:
i) the amount of the polymeric material is about 5 volume percent or more, based on the total volume of the coated fibers; or
ii) the polymeric material includes a thermoset polymer or thermoplastic polymer; or
iii) the coated fiber has a cross-section perpendicular to the length direction characterized by an aspect ratio of about 1.5 or more; or
iv) two or more of the filaments contact one another; or
v) the number of filaments in the coated fiber is sufficiently low so that the aligned filaments can be infiltrated with a polymeric material or a polymerizable resin while maintaining a filament volume fraction of about 60 volume percent or more; or
vi) the filaments are treated to reduce a surface energy of the filament; or
vii) the filaments are formed of E-glass or S-glass; or
viii) the filaments include a sizing or other treatment for improving adhesion to the polymeric material; or
ix) the coated fiber includes about 25 volume percent or less of organic fibers.

12. (canceled)

13. (canceled)

14. (canceled)

15. (canceled)

16. (canceled)

17. (canceled)

18. (canceled)

19. The coated fiber of claim 1, wherein the filaments are formed of glass filaments having a surface modified by ion swapping.

20. (canceled)

21. A fibrous bundle comprising two or more of the coated fibers of claim 1, and a polymeric material between the coated fibers, wherein the concentration of filaments in the fibrous bundle is about 55 volume percent or more, or wherein the coated fibers are aligned in an axial direction, and the fibrous bundle has a substantially uniform profile along its length in the axial direction.

22. (canceled)

23. (canceled)

24. A coated fiber adapted for use in a composite with a polymeric matrix comprising a plurality of aligned inorganic filaments, the coated fiber having a substantially uniform profile along its length, wherein the filaments have a polymeric or polymerizable coating over at least a portion of their surface, wherein the structure of the fiber and the materials of the filament and the coating are selected for providing one or more of the following characteristics:
i) the coated fiber has a tensile modulus of about 10 GPa or more, as measured according to ASTM D638-14, using a fiber shaped specimen; or
ii) the coated fiber has a flexural modulus of about 5 GPa or more, as measured according to ASTM D 790-17; or
iii) the coated fiber has a bending radius of about 10 cm or less, as measured according to ASTM E290-14 (semi-guided bend test around a mandrel);
wherein the concentration of filaments in the fibrous bundle is about 55 volume percent or more, wherein the coated fibers are aligned in an axial direction, and the fibrous bundle has a substantially uniform profile along its length in the axial direction; and
wherein the two or more coated fibers includes a first coated fiber having filaments twisted in a first directions and an adjacent second coated fiber including filaments twisted in a second direction reverse of the first direction.

25. The fibrous bundle of claim 21, wherein the filaments are impregnated with a thermo-formable polymeric material.

26. (canceled)

27. The fibrous bundle of claim 25,
wherein the fibrous bundle includes a polymeric material between two fibers, wherein the polymeric material is a same or different polymeric material as a polymeric material in the filaments; or the filaments extend a length of the fibrous bundle.

28. (canceled)

29. A fibrous composite comprising a plurality of fibrous bundles of any claim 21, wherein the fibrous bundles are attached or connected by a plurality of bias fiber elements that wrap, weave together, braid, interlace, or interlock the fibrous bundles.

30. The fibrous composite of claim 29,
wherein each of the bias fiber elements includes one or more axially aligned fibers; and/or
a first bias fiber element is angled in a first direction and a second bias fiber element is angled in a reverse direction relative to the axial direction of the fibrous bundles, optionally wherein the first bias fiber element is angled at 25° to 65° and the second bias fiber element is angle at −25° to −65°; or
wherein the bias fiber element includes two or more axially aligned fibers, wherein the bias fiber element has a length in an axial direction and a width and thickness in directions orthogonal to the axial direction, wherein a ratio of a thickness of the bias fiber element to a thickness of the fiber is about 2.8 or less; or
wherein the bias fiber elements are aligned at one or more angles having an absolute value of 10° or more relative to the axial direction of the fibrous bundles.

31. (canceled)

32. A coated fiber adapted for use in a composite with a polymeric matrix comprising a plurality of aligned inorganic filaments, the coated fiber having a substantially uniform profile along its length, wherein the filaments have a polymeric or polymerizable coating over at least a portion of their surface, wherein the structure of the fiber and the materials of the filament and the coating are selected for providing one or more of the following characteristics:
i) the coated fiber has a tensile modulus of about 10 GPa or more, as measured according to ASTM D638-14, using a fiber shaped specimen; or
ii) the coated fiber has a flexural modulus of about 5 GPa or more, as measured according to ASTM D 790-17; or
iii) the coated fiber has a bending radius of about 10 cm or less, as measured according to ASTM E290-14 (semi-guided bend test around a mandrel;
wherein
the concentration of filaments in the fibrous bundle is about 55 volume percent or more;
the coated fibers are aligned in an axial direction;
the fibrous bundle has a substantially uniform profile along its length in the axial direction;
wherein an aspect ratio of a width to a thickness of the bias fiber element is about 2 or more.

33. (canceled)

34. (canceled)

35. (canceled)

36. The fibrous composite of claim 29,
wherein the fibrous bundles are woven together by the bias fiber elements, wherein the woven structure has openings; or
wherein the fibrous composite has a sufficiently open structure so that a two or more of the fibrous bundles can cooperatively move relative to each other without fracturing the filaments and, or the fibers; or
wherein the number of fibrous bundles is 3 or more; or
wherein the number of fibrous bundles is about 10 or less; or
wherein the fibrous composite includes wicking channels.

37. (canceled)

38. (canceled)

39. (canceled)

40. (canceled)

41. (canceled)

42. A composite element comprising a core region including the fibrous composite of claim 29, at least partially covered by a second region including a polymeric covering formed of a polymeric material;
optionally wherein the polymeric material of the second region is a filled polymeric material;
optionally wherein the filled polymeric material includes particles of a filler that are biodegradable or bioresorbable;
optionally wherein the filled polymeric material is a thermoset material or a thermoplastic material;
optionally wherein the filled polymeric material has a melt flow rate of about viscosity of about 1 g/10 min or more as measured according to ASTM D1238-20 at 200° C./2.16 kg;
optionally wherein the polymeric material of the core regions (e.g., the coated fiber) has a viscosity of about the filled polymeric material has a melt flow rate of about viscosity of about 1 g/10 min or more as measured according to ASTM D1238-20 at 200° C./2.16 kg;
optionally wherein the composite element can have a bending radius, as measured according to ASTM E290-14, of about 20 cm or less at a bending angle of about 45°;
optionally wherein the composite element includes a plurality of the fibrous composites, wherein the fibrous composites are spaced apart in the core region and embedded in a polymeric matrix;
optionally wherein two or more of the fibrous composites are attached, connected, or positioned by a plurality of bias fiber elements that wrap, weave together, braid, interlace, or interlock the fibrous composites;
optionally wherein the composite element has a substantially uniform profile along its length and the filaments of the fibrous bundles extend a length of the composite element;
optionally wherein the core regions is covered by the polymeric material of the second region by a pultrusion process, where the coated core is passed through a die to form the desired profile;
optionally wherein the biodegradability or bioresorbability of the second region is different than that first region;
optionally wherein the composite element or the fibrous bundles has a non-circular cross-section so that the maximum packing can be increased;
optionally wherein the composite element is characterized by one or any combination of the following: a flexural modulus of about 20 GPa or more, as measured according to ASTM D 790-17, or a compressive strength of about 20 GPa or more, as measured according to ASTM D3410-16, or a compressive strain at failure of about 5% or more, as measured according to ASTM D3410-16, or a tensile strength of about 20 GPa or more, as measured according to ASTM D638-14; or a bending radius of about 20 cm or less, as measured according to ASTM E290-14.

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. An implant comprising:
i) the composite element claim 42; and
ii) a covering;
optionally wherein the implant is a screw or a pin, optionally a screw having a recessed end shaped for receiving a driving tool (recess may be in a head or in a shaft of a headless screw;
optionally wherein the covering includes a threaded outer surface;
optionally wherein the covering includes openings;
optionally wherein the covering has a rough or porous surface;
optionally wherein the covering is a polymeric material, optionally including one or more fillers,
optionally including one or more biodegradable or bioresorbable fillers;
optionally wherein the implant is capable of being torqued by a driver without failure;
optionally wherein the biodegradability or the bioresorbability of the covering is different than the composite element.

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. A kit for an implantable splint comprising:
a container for inserting into a medullary canal;
a plurality of the composite elements of claim 42, for packing into the container; and
a polymerizable resin for inserting into the container.

72. The kit of claim 71,
wherein the polymerizable resin polymerizes and/or cross-links at a temperature of about 30° C. to about 50° C.; or
wherein the container is a containment bag or other container that constrains the flow of the polymerizable resin and prevents or reduces blood or other bodily fluids from entering the container while the polymerizable resin is polymerizing and/or cross-linking; or
wherein the container is bioresorbable and/or biodegradable; or
wherein the container has a surface that promotes osteointegration; or
wherein the container is formed of a polymeric material; or
wherein the container is formed from fibers, optionally fibers that are woven, braided or knitted; or
wherein the kit includes a tube for inserting into the container; or
wherein the container is a double walled bag, so that a hole can be created through a portion or an entire length of the implant, optionally to connect the medullary canal above and below the implant; or
wherein the polymerizable resin includes a biodegradable or bioresorbable filler; or
wherein the kit includes about 6 or more of the composite elements having about the same length; or
wherein the composite elements has a bending radius of about 10 cm or less for inserting into a catheter or other flexed tube for delivering one or more of the composite elements into a medullary canal; or
wherein the kit includes a catheter for delivering one or more of the components into a medullar canal or other space, optionally wherein upon immersion in the salt water for an additional 7 weeks, the cylinders have a structure of interconnected pores.

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. A scaled (i.e., larger) composite element comprising two or more composite element of any of claim 42, optionally, wherein the composite elements are interlaced or interlocked or bounded by bias fiber elements, such as in a braiding.
